(19) United States
(12) Patent Application Publication
(10) Pub. No.: US 2022/0093871 A1
(43) Pub. Date: Mar. 24, 2022

(54) COMPOUND, MATERIAL FOR AN ORGANIC ELECTROLUMINESCENT DEVICE AND APPLICATION THEREOF

(71) Applicants: Wuhan Tianma Microelectronics Co., Ltd., Wuhan (CN); Wuhan Tianma Microelectronics Co., Ltd. Shanghai Branch, Shanghai (CN)

(72) Inventors: Wenpeng Dai, Wuhan (CN); Wei Gao, Wuhan (CN); Lu Zhai, Wuhan (CN); Lei Zhang, Wuhan (CN); Xia Li, Wuhan (CN)

(21) Appl. No.: 17/539,339

(22) Filed: Dec. 1, 2021

(30) Foreign Application Priority Data
Apr. 30, 2021 (CN) 202110482859.0

(51) Int. Cl. H01L 51/00; C07D 263/57; C09K 11/06; C07D 277/66

(52) U.S. Cl. CPC H01L 51/0061; C07D 263/57; H01L 51/5253; H01L 51/006; C07D 277/66; C09K 11/06

(57) ABSTRACT

Provided are a compound, a material for an organic electroluminescent device and an application thereof. The compound has a structure which is formed by substituting at any substitutable position of a structure represented by Formula I with at least one cyano group. The compound has a relatively high refractive index in the region of visible light (400-750 nm) and a reduced extinction coefficient K. When used in the organic electroluminescent device especially as a material for a capping layer, the compound can effectively improve the light extraction efficiency of an organic optoelectronic device, improve external quantum efficiency (EQE), implement display at various angles, and can effectively alleviate a color cast.

### CROSS-REFERENCE TO RELATED APPLICATION(S)

This application claims priority to Chinese Patent Application No. 202110482859.0 filed Apr. 30, 2021, the disclosure of which is incorporated herein by reference in its entirety.

### TECHNICAL FIELD

The present disclosure relates to the field of organic electroluminescence and, in particular, to a compound, a material for an organic electroluminescent device and an application thereof.

### BACKGROUND

After decades of development, organic electroluminescence (such as organic light-emitting diode (OLED)) has gained considerable progress. The OLED has internal quantum efficiency of approximately 100% and external quantum efficiency of only about 20%. Most light is confined inside a light-emitting device due to factors such as a loss of a substrate mode, a surface plasmon loss and a waveguide effect, resulting in the loss of a large amount of energy.

In a top emitting device, an organic capping layer (CPL) is deposited through evaporation on a translucent metal electrode Al so that an optical interference distance is adjusted, the reflection of external light is suppressed, and the extinction caused by the movement of surface plasmon is suppressed, thereby improving light extraction efficiency and light-emitting efficiency.

High requirements are imposed on the performance of a material for the CPL: no absorption within the wavelength range (400 nm to 700 nm) of visible light, a high refractive index (generally, n>2.1), a low extinction coefficient (k≤0.00) within the wavelength range of 400 nm to 600 nm, a high glass transition temperature, high molecular thermal stability, and an ability to be deposited through evaporation without thermal decomposition.

Materials for the CPL in the related art still have many problems, for example, (1) a refractive index is generally below 1.9 and cannot meet the requirement for the high refractive index; (2) in the case where the refractive index meets the requirement, the materials have relatively strong absorption or a relatively large extinction coefficient in the region of visible light; (3) amine derivatives with a particular structure and a high refractive index and the use of materials that have particular parameters have improved the light extraction efficiency, while the problems of light-emitting efficiency and chromaticity are still to be solved especially for blue light-emitting elements; (4) to increase the density of molecules and achieve high thermal stability, a molecular structure is designed to be large and loose so that molecules cannot be tightly packed, resulting in too many molecular gel holes during evaporation and incomplete coverage; (5) a simple design of an electron-type capping layer material to achieve the effects of electron transmission and light extraction saves a preparation cost of the device to a certain extent so that multiple effects are achieved, while the design is not conducive to light extraction and improves the light-emitting efficiency only slightly and the problem of chromaticity is not solved.

Therefore, more kinds of CPL materials with higher performance are to be developed in the art.

### SUMMARY

In view of defects in the related art, a first object of the present disclosure is to provide a compound, and in particular a material for a capping layer. When used in an organic electroluminescent device especially as the material for the capping layer, the compound can effectively improve the light-emitting efficiency of the device and is conducive to absorbing harmful light and protecting eyesight.

To achieve the object, the present disclosure adopts a solution described below.

The present disclosure provides a compound having a structure which is formed by substituting at any substitutable position of a structure represented by Formula I with at least one cyano group:

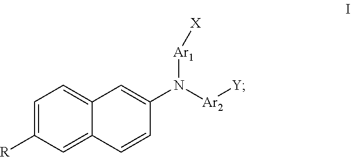

wherein

in Formula I, R is selected from any one of substituted or unsubstituted fused ring C10-C60 aryl or substituted or unsubstituted fused ring C6-C60 heteroaryl;

in Formula I, Ar₁ and Ar₂ are each independently selected from any one of a single bond, substituted or unsubstituted C6-C60 arylene, substituted or unsubstituted C3-C60 heteroarylene, substituted or unsubstituted fused ring C10-C60 arylene or substituted or unsubstituted fused ring C6-C60 heteroarylene; and

in Formula I, X and Y are each independently selected from substituted or unsubstituted C6-C60 aryl or substituted or unsubstituted C3-C60 heteroaryl, and at least one of X or Y is selected from substituted or unsubstituted C3-C60 electron withdrawing heteroaryl;

wherein substituents in R, Ar₁, Ar₂, X and Y are each independently selected from any one or a combination of at least two of protium, deuterium, tritium, halogen, C1-C10 alkyl, C1-C10 haloalkyl, C1-C10 alkoxy, C6-C60 aryl and C3-C60 heteroaryl.

A second object of the present disclosure is to provide a material for an organic electroluminescent device. The material for the organic electroluminescent device includes any one or a combination of at least two of the compounds as described for the first object.

A third object of the present disclosure is to provide an organic electroluminescent device. The organic electroluminescent device includes a first electrode layer, an organic function layer and a second electrode layer which are stacked in sequence;

wherein the organic function layer includes the material as described for the second object.

A fourth object of the present disclosure is to provide an organic electroluminescent device. The organic electroluminescent device includes a first capping layer, a first electrode layer, an organic function layer and a second electrode layer which are stacked in sequence;

wherein the first capping layer includes the material as described for the second object.

A fifth object of the present disclosure is to provide a display panel. The display panel includes the organic electroluminescent device as described for the third object or the fourth object.

A sixth object of the present disclosure is to provide a display device. The display device includes the display panel as described for the fifth object.

Compared with the related art, the present disclosure has beneficial effects described below.

(1) The compound provided by the present disclosure has a relatively high refractive index in the region of visible light (400-750 nm). When used in the organic electroluminescent device especially as the material for the capping layer, the compound can effectively improve the light extraction efficiency of an organic optoelectronic device, improve external quantum efficiency (EQE), implement display at various angles, and can effectively alleviate a color cast.

(2) The compound of the present disclosure has a relatively large extinction coefficient in the ultraviolet region (less than 400 nm), which is conducive to absorbing harmful light and protecting eyesight.

(3) The compound of the present disclosure has a relatively small extinction coefficient in the region of blue light (400-450 nm) and hardly absorbs blue light, which is conducive to improving the light-emitting efficiency.

### BRIEF DESCRIPTION OF DRAWINGS

FIG. 1 is a structure diagram of an organic electroluminescent device according to an embodiment of the present disclosure.

FIG. 2 is a structure diagram of an organic electroluminescent device according to another embodiment of the present disclosure.

### REFERENCE LIST

1 substrate
2 anode
3 hole injection layer
4 first hole transport layer
5 second hole transport layer
6 light-emitting layer
7 first electron transport layer
8 second electron transport layer
9 cathode
10 first capping layer
11 second capping layer

### DETAILED DESCRIPTION

For a better understanding of the present disclosure, examples of the present disclosure are listed below. It is to be understood by those skilled in the art that the examples described herein are used for a better understanding of the present disclosure and not to be construed as specific limitations to the present disclosure.

A first object of the present disclosure is to provide a compound. The compound has a structure which is formed by substituting at any substitutable position of a structure represented by Formula I with at least one cyano group:

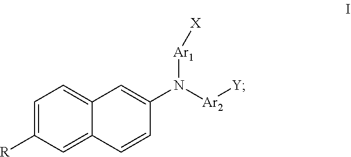

wherein

in Formula I, R is selected from any one of substituted or unsubstituted fused ring C10-C60 (for example, C10, C12, C14, C16, C18, C20, C22, C24, C26, C28, C30, C32, C34, C36, C38, C40, C42, C44, C46, C48, C50, C52, C54, C56, C58 or the like) aryl or substituted or unsubstituted fused ring C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) heteroaryl;

in Formula I, Ar₁ and Ar₂ are each independently selected from any one of a single bond, substituted or unsubstituted C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) arylene, substituted or unsubstituted C3-C60 (for example, C4, C5, C6, C7, C8, C10, C12, C14, C16, C18, C20, C22, C24, C26, C28, C30, C32, C34, C36, C38, C40, C42, C44, C46, C48, C50, C52, C54, C56, C58 or the like) heteroarylene, substituted or unsubstituted fused ring C10-C60 (for example, C10, C12, C14, C16, C18, C20, C22, C24, C26, C28, C30, C32, C34, C36, C38, C40, C42, C44, C46, C48, C50, C52, C54, C56, C58 or the like) arylene or substituted or unsubstituted fused ring C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) heteroarylene; and

in Formula I, X and Y are each independently selected from substituted or unsubstituted C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) aryl or substituted or unsubstituted C3-C60 (for example, C4, C5, C6, C7, C8, C10, C12, C14, C16, C18, C20, C22, C24, C26, C28, C30, C32, C34, C36, C38, C40, C42, C44, C46, C48, C50, C52, C54, C56, C58 or the like) heteroaryl, and at least one of X or Y is selected from substituted or unsubstituted C3-C60 electron withdrawing heteroaryl;

wherein substituents in R, Ar₁, Ar₂, X and Y are each independently selected from any one or a combination of at least two of protium, deuterium, tritium, halogen, C1-C10 (for example, C2, C3, C4, C5, C6, C7, C8, C9 or the like) alkyl, C1-C10 (for example, C2, C3, C4, C5, C6, C7, C8, C9 or the like) haloalkyl, C1-C10 (for example, C2, C3, C4, C5, C6, C7, C8, C9 or the like) alkoxy, C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) aryl or C3-C60 (for example, C4, C5, C6, C7, C8, C10, C12, C14, C16, C18, C20, C22, C24, C26, C28, C30, C32, C34, C36, C38, C40, C42, C44, C46, C48, C50, C52, C54, C56, C58 or the like) heteroaryl.

The researchers of the present disclosure have found that in the compound structure formed through the substitution of an arylamino group at position 2 and the substitution of a fused ring group (R) at position 6 on a naphthalene ring, the naphthalene conjugation is the longest, smallest molecular distortion is formed, and a film formed through molecular evaporation has a smoother arrangement. Meanwhile, the substitution of a cyano group in the structure can resonate with a distant heterocyclic structure, which significantly changes molecular polarity and greatly improves light extraction efficiency. Therefore, the compound of the present disclosure facilitates the internal extraction of visible light. The compound has a higher refractive index in the region of visible light, is suitable for use as a material for a capping layer of an organic electroluminescent device, and can effectively improve the light extraction efficiency and external quantum efficiency. Moreover, the compound has a relatively large extinction coefficient in an ultraviolet region (less than 400 nm), thereby facilitating the absorption of harmful light. The compound has a relatively small extinction coefficient in the region of blue light (400-450 nm) and hardly absorbs blue light, which is conducive to improving the light-emitting efficiency.

In addition, the introduction of the cyano group can change the electron cloud distribution of molecules, reduce the absorption coefficient K of molecules, reduce the absorption of blue light by the device, and help increase the efficiency of blue light-emitting devices.

In an embodiment, the number of the cyano group(s) in the compound is 1 to 10, for example, 2, 3, 4, 5, 6, 7, 8, 9 or the like, preferably 1 to 3.

In the present disclosure, the number of the cyano substituent(s) is preferably not greater than 10, further preferably not greater than 3. Though the substitution of cyano groups is conducive to increasing the refractive index, an increase of the number of substitutions to a certain limit will not significantly increase the refractive index, and too many substitutions will lead to the reduced thermal stability of the material.

In an embodiment, at least one cyano substituent is on the group R.

In the present disclosure, the cyano substituent is preferably on the group R. Such a structure has the best resonance effect and thus is more conducive to increasing a molecular polarization ratio and improving the light extraction efficiency of molecules.

In an embodiment, at least one cyano substituent is on Ar₁, Ar₂, X or Y.

In an embodiment, the compound has a structure represented by Formula (1), Formula (2), Formula (3) or Formula (4):

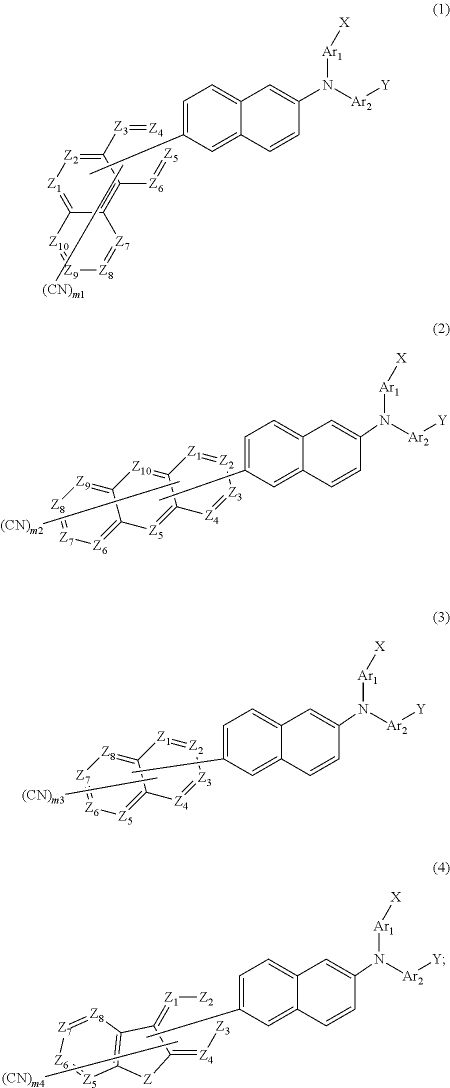

wherein

m1 and m2 are each independently selected from an integer from 1 to 9, for example, 2, 3, 4, 5, 6, 7, 8 or the like, and m3 and m4 are each independently selected from an integer from 1 to 7, for example, 2, 3, 4, 5, 6, 7 or the like;

Z is selected from 0 or S;

Z₁ to Z₁₀ are each independently selected from a N atom, CH or CR₁, where R₁ is selected from any one or a combination of at least two of protium, deuterium, tritium, halogen, C1-C10 (for example, C2, C3, C4, C5, C6, C7, C8, C9 or the like) alkyl, C1-C10 (for example, C2, C3, C4, C5, C6, C7, C8, C9 or the like) haloalkyl, C1-C10 (for example, C2, C3, C4, C5, C6, C7, C8, C9 or the like) alkoxy, C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) aryl or C3-C60 (for example, C4, C5, C6, C7, C8, C10, C12, C14, C16, C18, C20, C22, C24, C26, C28, C30, C32, C34, C36, C38, C40, C42, C44, C46, C48, C50, C52, C54, C56, C58 or the like) heteroaryl; and

Ar₁, Ar₂, X and Y each have the same selection range as defined in Formula I.

In an embodiment, the compound has any one of the following structures:

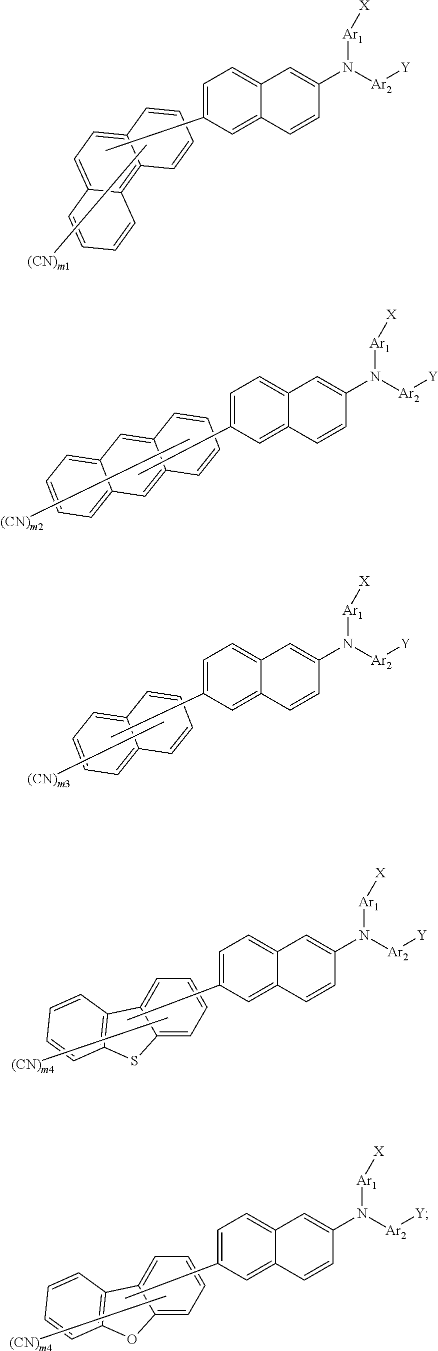

wherein

m1 and m2 are each independently selected from an integer from 1 to 9, for example, 2, 3, 4, 5, 6, 7, 8 or the like, and m3 and m4 are each independently selected from an integer from 1 to 7, for example, 2, 3, 4, 5, 6, 7 or the like; and

Ar₁, Ar₂, X and Y each have the same selection range as defined in Formula I.

In an embodiment, the compound has any one of the following structures:

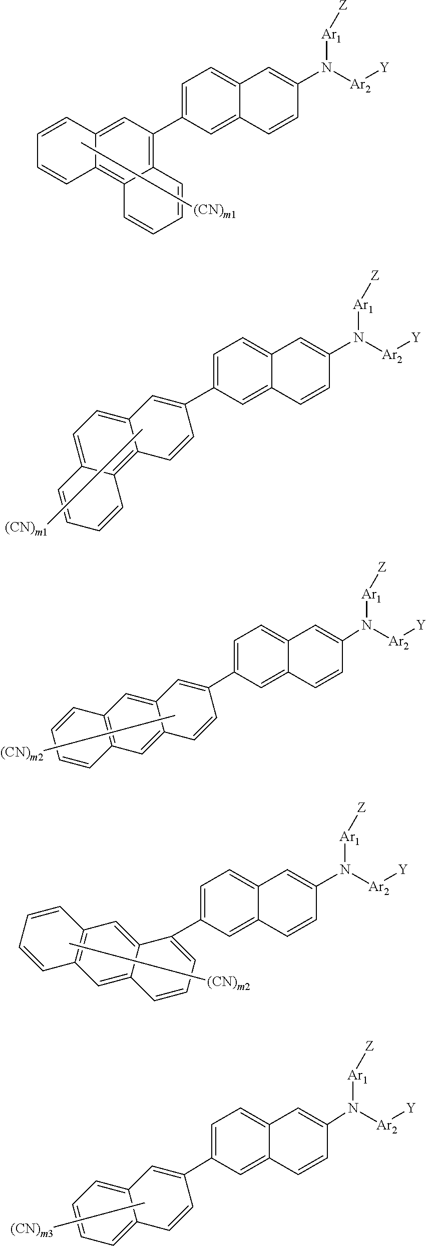

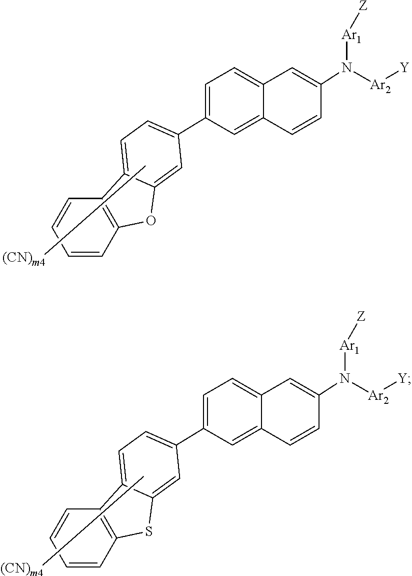

wherein

m1 and m2 are each independently selected from an integer from 1 to 9, for example, 2, 3, 4, 5, 6, 7, 8 or the like, and m3 and m4 are each independently selected from an integer from 1 to 7, for example, 2, 3, 4, 5, 6 or the like; and

Ar₁, Ar₂, X and Y each have the same selection range as defined in Formula I.

In an embodiment, R is selected from any one of groups represented by Formula (5), Formula (6) or Formula (7):

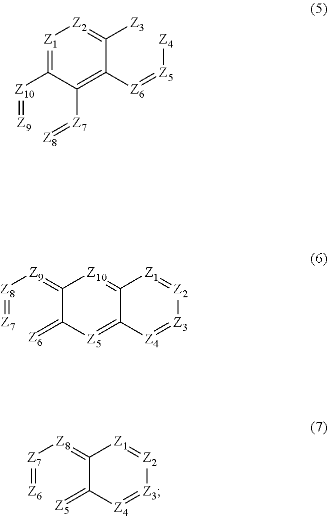

wherein

Z₁ to Z₁₀ are each independently selected from a N atom, CH or CR₁, where R₁ is selected from any one or a combination of at least two of protium, deuterium, tritium, halogen, C1-C10 (for example, C2, C3, C4, C5, C6, C7, C8, C9 or the like) alkyl, C1-C10 (for example, C2, C3, C4, C5, C6, C7, C8, C9 or the like) haloalkyl, C1-C10 (for example, C2, C3, C4, C5, C6, C7, C8, C9 or the like) alkoxy, C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) aryl or C3-C60 (for example, C4, C5, C6, C7, C8, C10, C12, C14, C16, C18, C20, C22, C24, C26, C28, C30, C32, C34, C36, C38, C40, C42, C44, C46, C48, C50, C52, C54, C56, C58 or the like) heteroaryl.

In a preferred technical solution of the present disclosure, R is a structure where at most three rings are fused so that not only a good light extraction effect but also high thermal stability can be achieved. A structure with too many fused rings results in thermal decomposition and poor solubility, which is unfavorable for cleaning a MASK during mass production.

In an embodiment, R is selected from any one of the following groups:

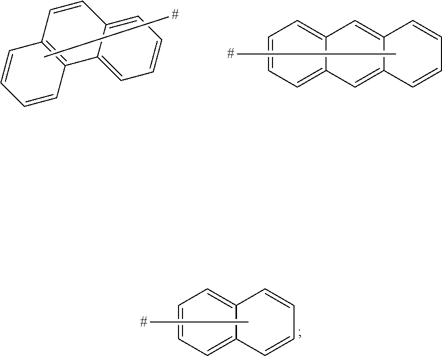

wherein

# represents a linkage site of the group.

In an embodiment, R is selected from any one of the following groups:

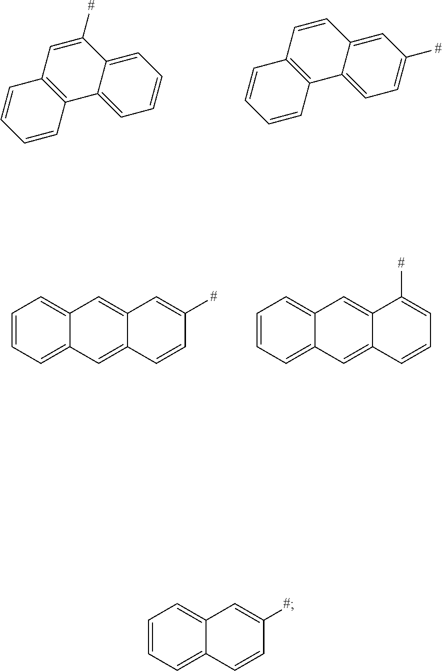

wherein

# represents a linkage site of the group.

In the present disclosure, naphthyl, anthracenyl and phenanthrenyl are preferably linked at the preceding sites so that a molecule of the compound has a high refractive index due to the longest conjugated chain.

In an embodiment, X and Y are each independently selected from substituted or unsubstituted C3-C60 electron withdrawing heteroaryl.

In an embodiment, X and Y are each independently selected from any one of the following substituted or unsubstituted groups:

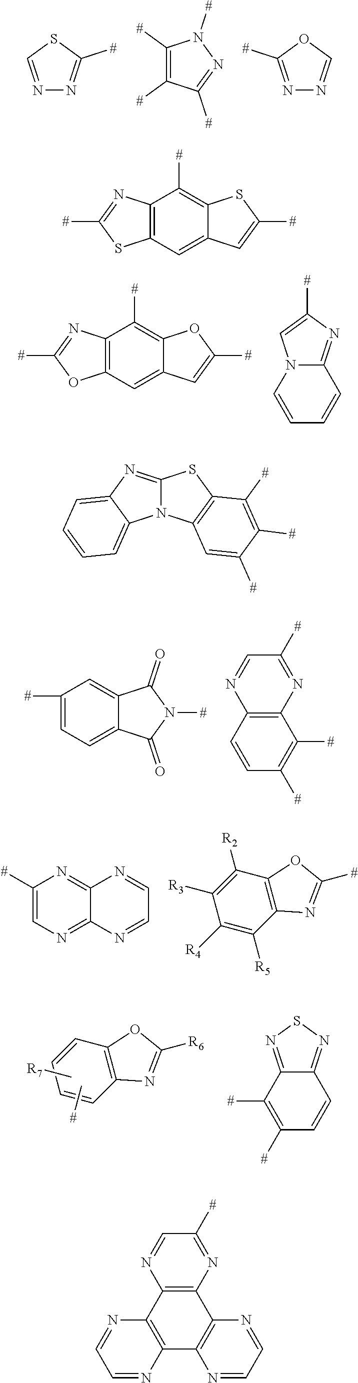

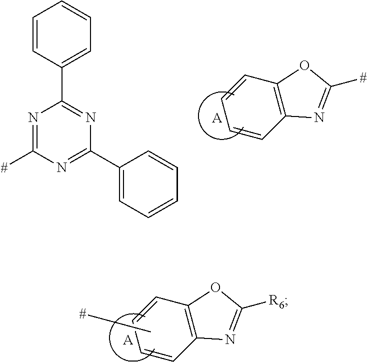

wherein # represents a linkage site of the group;

Q is selected from an O atom, a S atom or NR_{B};

R₂ to R₈ are each independently selected from any one of hydrogen, protium, deuterium, tritium, halogen, C1-C10 (for example, C2, C3, C4, C5, C6, C7, C8, C9 or the like) alkyl, C1-C10 (for example, C2, C3, C4, C5, C6, C7, C8, C9 or the like) alkoxy, C6-C60 (for example, C6, C7, C8, C10, C12, C14, C16, C18, C20, C22, C24, C26, C28, C30, C32, C34, C36, C38, C40, C42, C44, C46, C48, C50, C52, C54, C56, C58 or the like) aryl or C3-C60 (for example, C4, C5, C6, C7, C8, C10, C12, C14, C16, C18, C20, C22, C24, C26, C28, C30, C32, C34, C36, C38, C40, C42, C44, C46, C48, C50, C52, C54, C56, C58 or the like) heteroaryl; and

the ring A is fused at any available position of the benzene ring and the ring A is selected from substituted or unsubstituted C6-C30 (for example, C6, C7, C8, C10, C12, C14, C16, C18, C20, C22, C24, C26, C28 or the like) aromatic rings or substituted or unsubstituted C3-C30 (for example, C4, C5, C6, C7, C8, C10, C12, C14, C16, C18, C20, C22, C24, C26, C28 or the like) heteroaromatic rings.

In an embodiment, X and Y are each independently selected from any one of the following groups:

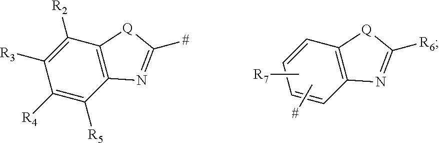

wherein

# represents a linkage site of the group; and

Q and R₂ to R₇ each have the same selection range as defined above.

In an embodiment, X and Y are each independently selected from any one of the following groups:

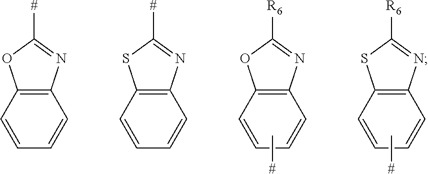

wherein

# represents a linkage site of the group; and

R₆ has the same selection range as defined above.

In an embodiment, Ar₁ and Ar₂ are each independently selected from any one of phenylene, biphenylene, terphenylene, naphthylene, anthrylene, phenanthrenylene, pyridinylene, pyrimidinylene, triazinylene, furylene, pyrrolidene, thienylene, quinolylene, isoquinolylene, benzofurylene or benzothienylene.

In an embodiment, the compound has any one of the following structures represented by P1 to P102:

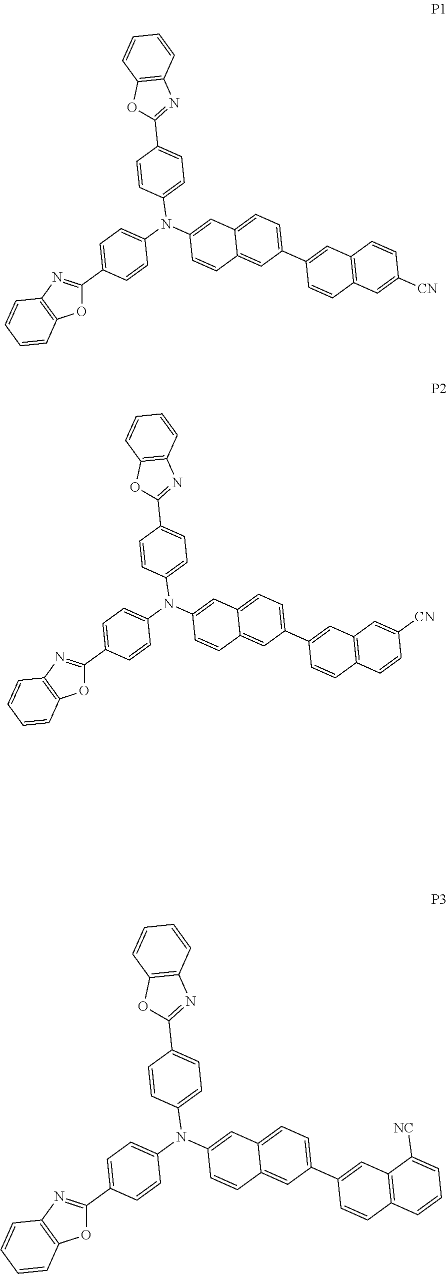 [SMILES: N#Cc1ccc2cc(-c3ccc4cc(N(c5ccc(-c6nc7ccccc7o6)cc5)c5ccc(-c6nc7ccccc7o6)cc5)ccc4c3)ccc2c1 ; N#Cc1ccc2ccc(-c3ccc4cc(N(c5ccc(-c6nc7ccccc7o6)cc5)c5ccc(-c6nc7ccccc7o6)cc5)ccc4c3)cc2c1 ; N#Cc1cccc2ccc(-c3ccc4cc(N(c5ccc(-c6nc7ccccc7o6)cc5)c5ccc(-c6nc7ccccc7o6)cc5)ccc4c3)cc12]

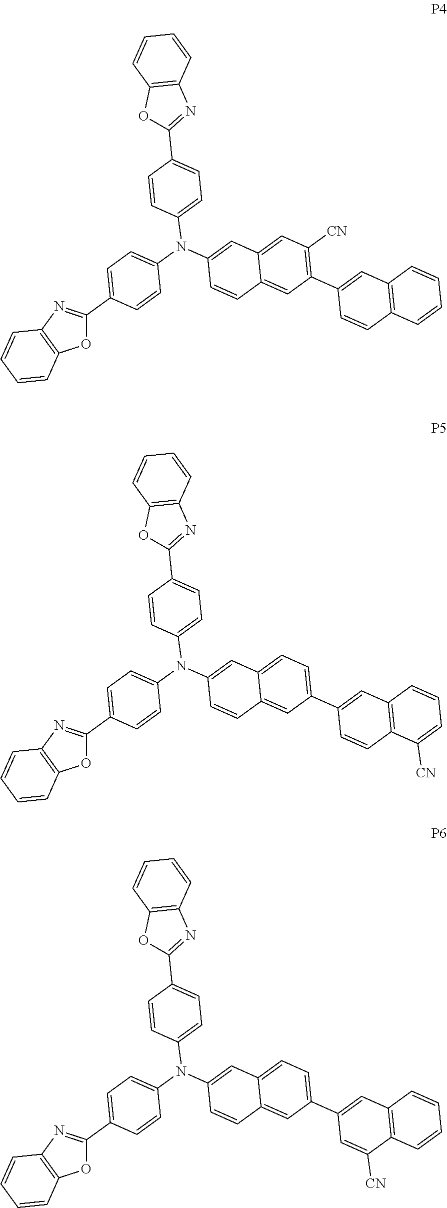 [SMILES: N#Cc1cc2cc(N(c3ccc(-c4nc5ccccc5o4)cc3)c3ccc(-c4nc5ccccc5o4)cc3)ccc2cc1-c1ccc2ccccc2c1 ; N#Cc1cccc2cc(-c3ccc4cc(N(c5ccc(-c6nc7ccccc7o6)cc5)c5ccc(-c6nc7ccccc7o6)cc5)ccc4c3)ccc12 ; N#Cc1cc(-c2ccc3cc(N(c4ccc(-c5nc6ccccc6o5)cc4)c4ccc(-c5nc6ccccc6o5)cc4)ccc3c2)cc2ccccc12]

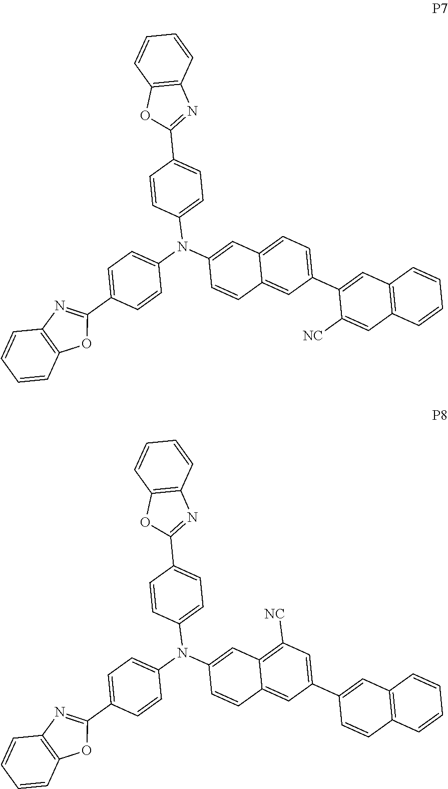 [SMILES: N#Cc1cc2ccccc2cc1-c1ccc2cc(N(c3ccc(-c4nc5ccccc5o4)cc3)c3ccc(-c4nc5ccccc5o4)cc3)ccc2c1 ; N#Cc1cc(-c2ccc3ccccc3c2)cc2ccc(N(c3ccc(-c4nc5ccccc5o4)cc3)c3ccc(-c4nc5ccccc5o4)cc3)cc12]

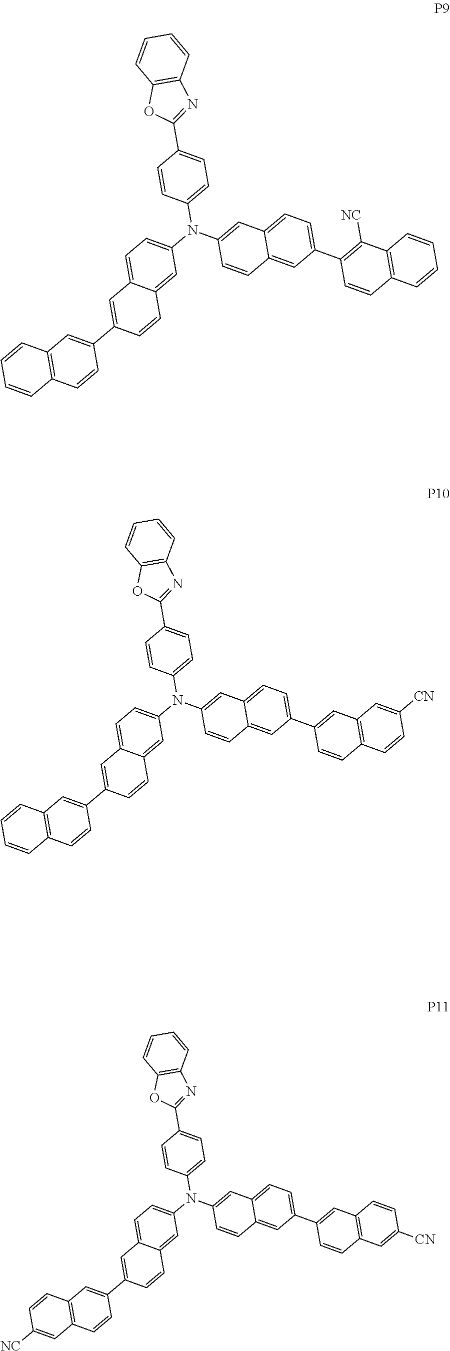 [SMILES: N#Cc1c(-c2ccc3cc(N(c4ccc(-c5nc6ccccc6o5)cc4)c4ccc5cc(-c6ccc7ccccc7c6)ccc5c4)ccc3c2)ccc2ccccc12 ; N#Cc1ccc2ccc(-c3ccc4cc(N(c5ccc(-c6nc7ccccc7o6)cc5)c5ccc6cc(-c7ccc8ccccc8c7)ccc6c5)ccc4c3)cc2c1 ; N#Cc1ccc2cc(-c3ccc4cc(N(c5ccc(-c6nc7ccccc7o6)cc5)c5ccc6cc(-c7ccc8cc(C#N)ccc8c7)ccc6c5)ccc4c3)ccc2c1]

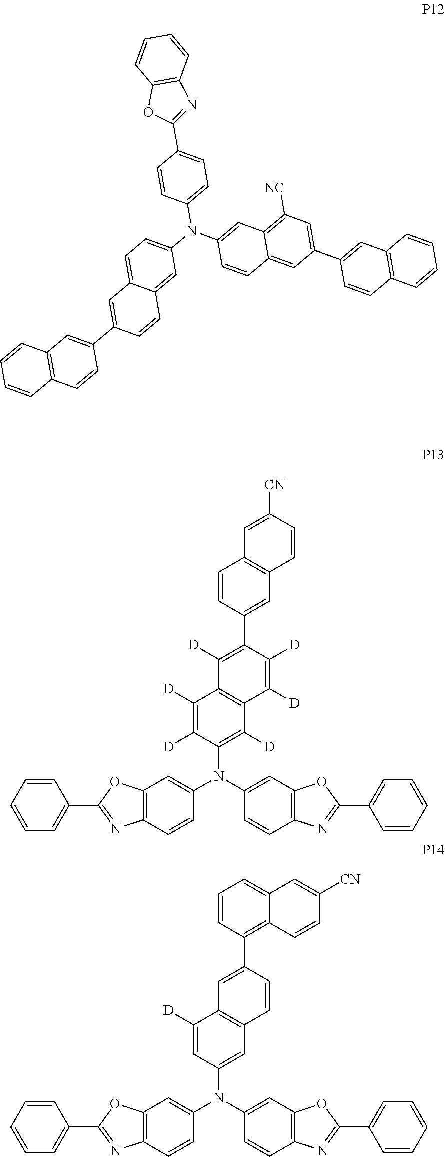 [SMILES: N#Cc1cc(-c2ccc3ccccc3c2)cc2ccc(N(c3ccc(-c4nc5ccccc5o4)cc3)c3ccc4cc(-c5ccc6ccccc6c5)ccc4c3)cc12 ; [2H]c1c(N(c2ccc3nc(-c4ccccc4)oc3c2)c2ccc3nc(-c4ccccc4)oc3c2)c([2H])c2c([2H])c([2H])c(-c3ccc4cc(C#N)ccc4c3)c([2H])c2c1[2H] ; [2H]c1cc(N(c2ccc3nc(-c4ccccc4)oc3c2)c2ccc3nc(-c4ccccc4)oc3c2)cc2ccc(-c3cccc4cc(C#N)ccc34)cc12]

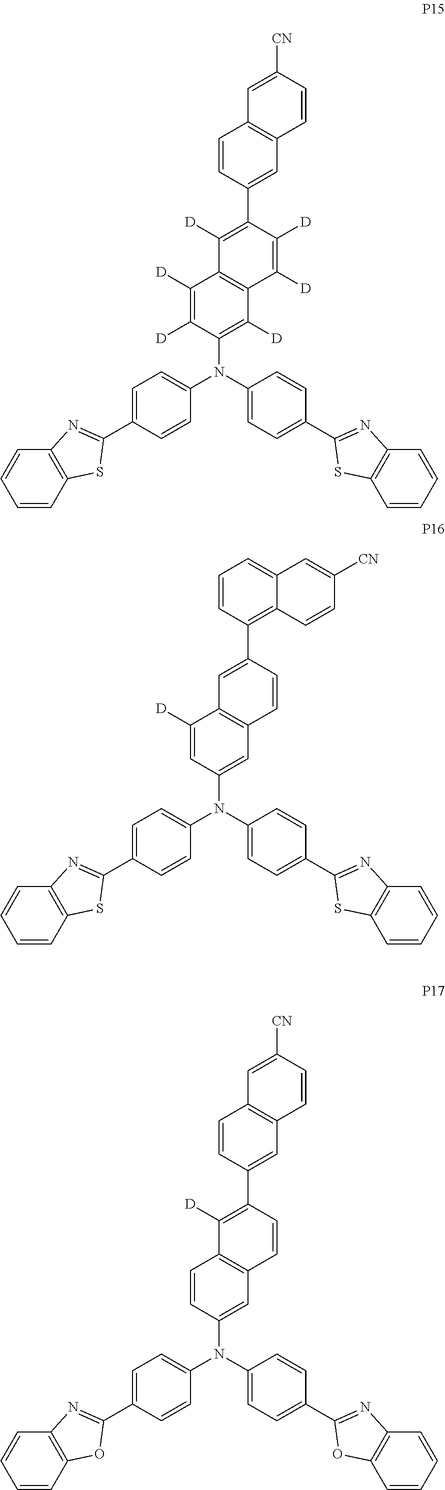 [SMILES: [2H]c1c(N(c2ccc(-c3nc4ccccc4s3)cc2)c2ccc(-c3nc4ccccc4s3)cc2)c([2H])c2c([2H])c([2H])c(-c3ccc4cc(C#N)ccc4c3)c([2H])c2c1[2H] ; [2H]c1cc(N(c2ccc(-c3nc4ccccc4s3)cc2)c2ccc(-c3nc4ccccc4s3)cc2)cc2ccc(-c3cccc4cc(C#N)ccc34)cc12 ; [2H]c1c(-c2ccc3cc(C#N)ccc3c2)ccc2cc(N(c3ccc(-c4nc5ccccc5o4)cc3)c3ccc(-c4nc5ccccc5o4)cc3)ccc12]

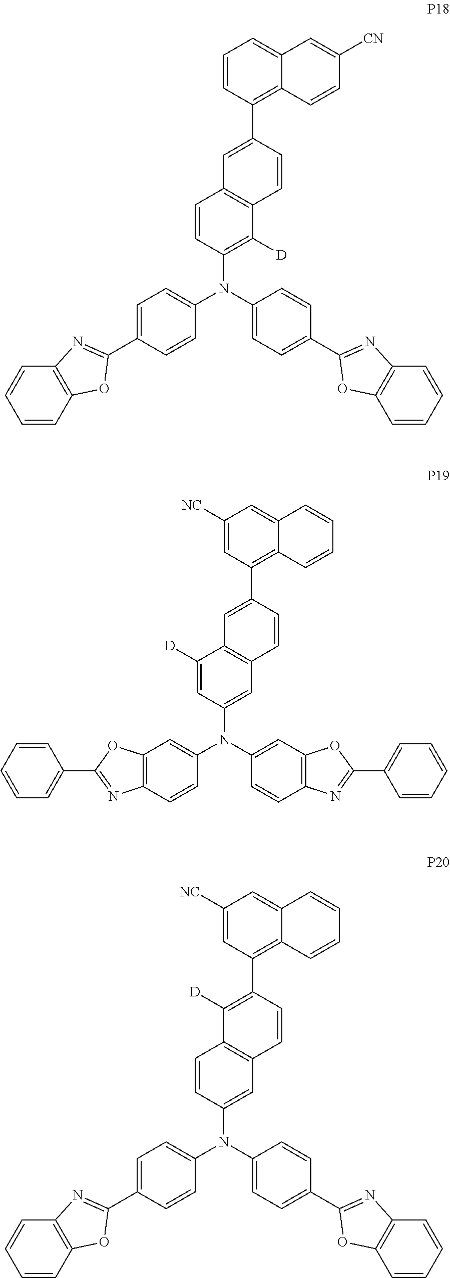 [SMILES: [2H]c1c(N(c2ccc(-c3nc4ccccc4o3)cc2)c2ccc(-c3nc4ccccc4o3)cc2)ccc2cc(-c3cccc4cc(C#N)ccc34)ccc12 ; [2H]c1cc(N(c2ccc3nc(-c4ccccc4)oc3c2)c2ccc3nc(-c4ccccc4)oc3c2)cc2ccc(-c3cc(C#N)cc4ccccc34)cc12 ; [2H]c1c(-c2cc(C#N)cc3ccccc23)ccc2cc(N(c3ccc(-c4nc5ccccc5o4)cc3)c3ccc(-c4nc5ccccc5o4)cc3)ccc12]

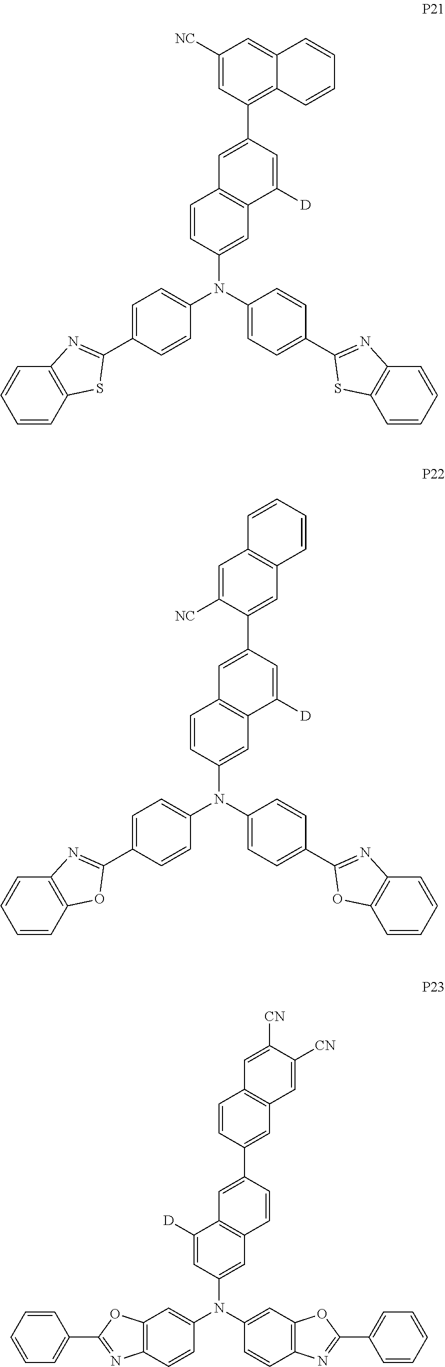 [SMILES: [2H]c1cc(-c2cc(C#N)cc3ccccc23)cc2ccc(N(c3ccc(-c4nc5ccccc5s4)cc3)c3ccc(-c4nc5ccccc5s4)cc3)cc12 ; [2H]c1cc(-c2cc3ccccc3cc2C#N)cc2ccc(N(c3ccc(-c4nc5ccccc5o4)cc3)c3ccc(-c4nc5ccccc5o4)cc3)cc12 ; [2H]c1cc(N(c2ccc3nc(-c4ccccc4)oc3c2)c2ccc3nc(-c4ccccc4)oc3c2)cc2ccc(-c3ccc4cc(C#N)c(C#N)cc4c3)cc12]

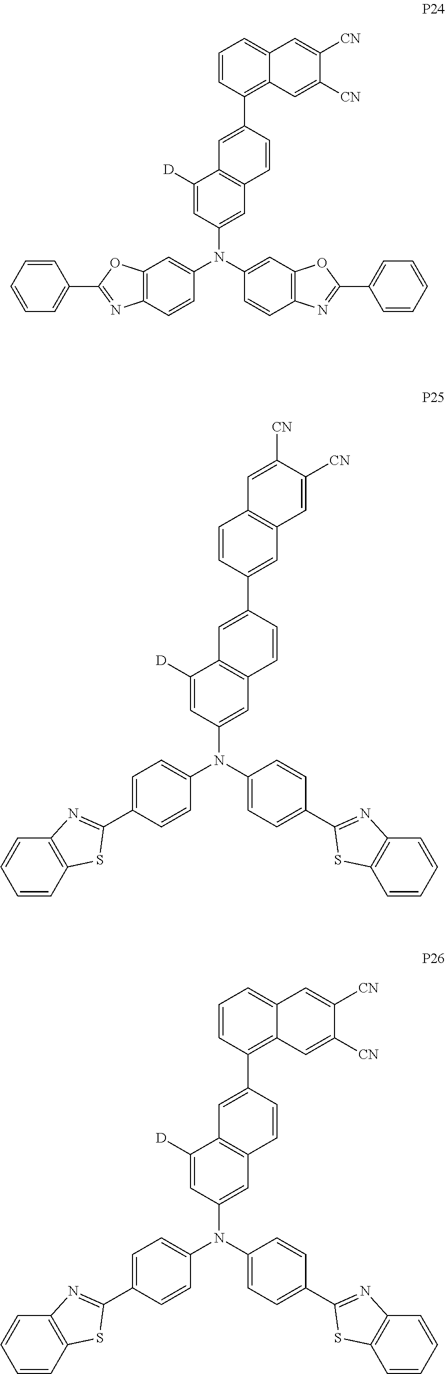 [SMILES: [2H]c1cc(N(c2ccc3nc(-c4ccccc4)oc3c2)c2ccc3nc(-c4ccccc4)oc3c2)cc2ccc(-c3cccc4cc(C#N)c(C#N)cc34)cc12 ; [2H]c1cc(N(c2ccc(-c3nc4ccccc4s3)cc2)c2ccc(-c3nc4ccccc4s3)cc2)cc2ccc(-c3ccc4cc(C#N)c(C#N)cc4c3)cc12 ; [2H]c1cc(N(c2ccc(-c3nc4ccccc4s3)cc2)c2ccc(-c3nc4ccccc4s3)cc2)cc2ccc(-c3cccc4cc(C#N)c(C#N)cc34)cc12]

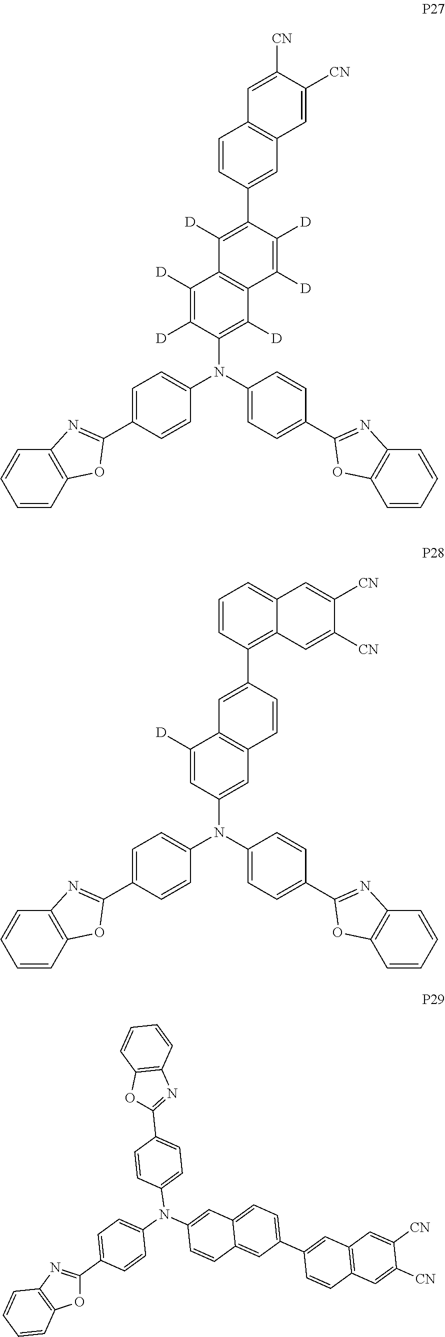 [SMILES: [2H]c1c(N(c2ccc(-c3nc4ccccc4o3)cc2)c2ccc(-c3nc4ccccc4o3)cc2)c([2H])c2c([2H])c([2H])c(-c3ccc4cc(C#N)c(C#N)cc4c3)c([2H])c2c1[2H] ; [2H]c1cc(N(c2ccc(-c3nc4ccccc4o3)cc2)c2ccc(-c3nc4ccccc4o3)cc2)cc2ccc(-c3cccc4cc(C#N)c(C#N)cc34)cc12 ; N#Cc1cc2ccc(-c3ccc4cc(N(c5ccc(-c6nc7ccccc7o6)cc5)c5ccc(-c6nc7ccccc7o6)cc5)ccc4c3)cc2cc1C#N]

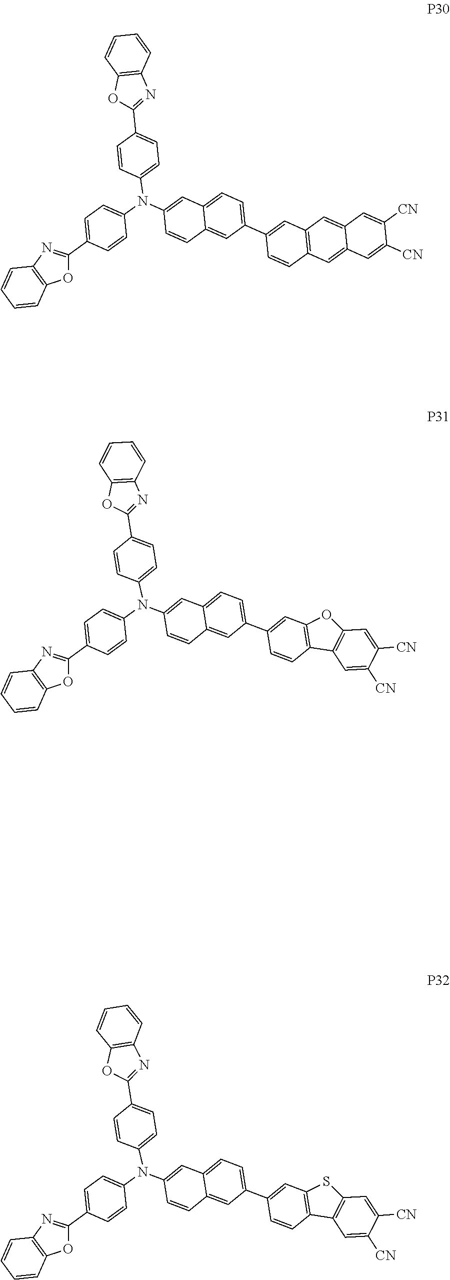

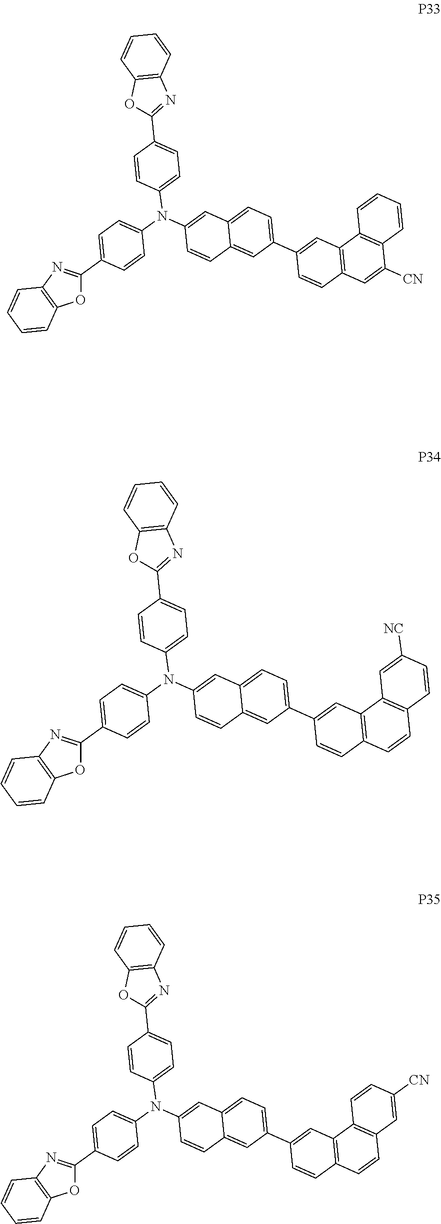 [SMILES: N#Cc1cc2ccc(-c3ccc4cc(N(c5ccc(-c6nc7ccccc7o6)cc5)c5ccc(-c6nc7ccccc7o6)cc5)ccc4c3)cc2c2ccccc12 ; N#Cc1ccc2ccc3ccc(-c4ccc5cc(N(c6ccc(-c7nc8ccccc8o7)cc6)c6ccc(-c7nc8ccccc8o7)cc6)ccc5c4)cc3c2c1 ; N#Cc1ccc2c(ccc3ccc(-c4ccc5cc(N(c6ccc(-c7nc8ccccc8o7)cc6)c6ccc(-c7nc8ccccc8o7)cc6)ccc5c4)cc32)c1]

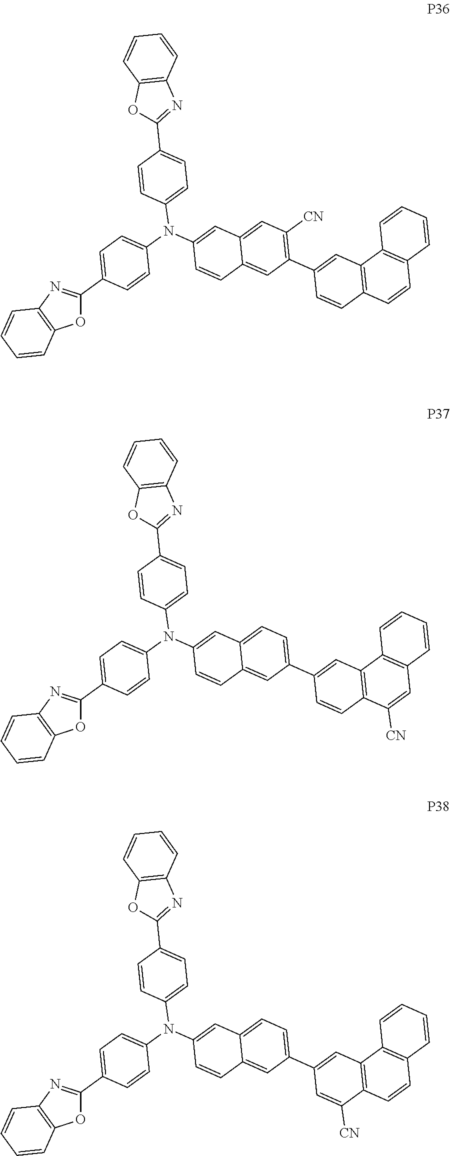 [SMILES: N#Cc1cc2cc(N(c3ccc(-c4nc5ccccc5o4)cc3)c3ccc(-c4nc5ccccc5o4)cc3)ccc2cc1-c1ccc2ccc3ccccc3c2c1 ; N#Cc1cc2ccccc2c2cc(-c3ccc4cc(N(c5ccc(-c6nc7ccccc7o6)cc5)c5ccc(-c6nc7ccccc7o6)cc5)ccc4c3)ccc12 ; N#Cc1cc(-c2ccc3cc(N(c4ccc(-c5nc6ccccc6o5)cc4)c4ccc(-c5nc6ccccc6o5)cc4)ccc3c2)cc2c1ccc1ccccc12]

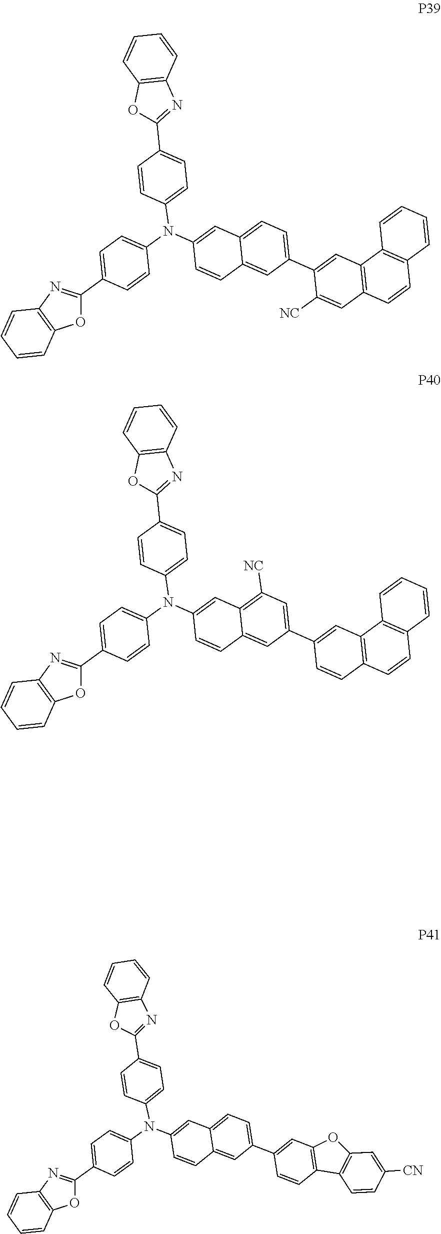 [SMILES: N#Cc1cc2ccc3ccccc3c2cc1-c1ccc2cc(N(c3ccc(-c4nc5ccccc5o4)cc3)c3ccc(-c4nc5ccccc5o4)cc3)ccc2c1 ; N#Cc1cc(-c2ccc3ccc4ccccc4c3c2)cc2ccc(N(c3ccc(-c4nc5ccccc5o4)cc3)c3ccc(-c4nc5ccccc5o4)cc3)cc12 ; N#Cc1ccc2c(c1)oc1cc(-c3ccc4cc(N(c5ccc(-c6nc7ccccc7o6)cc5)c5ccc(-c6nc7ccccc7o6)cc5)ccc4c3)ccc12]

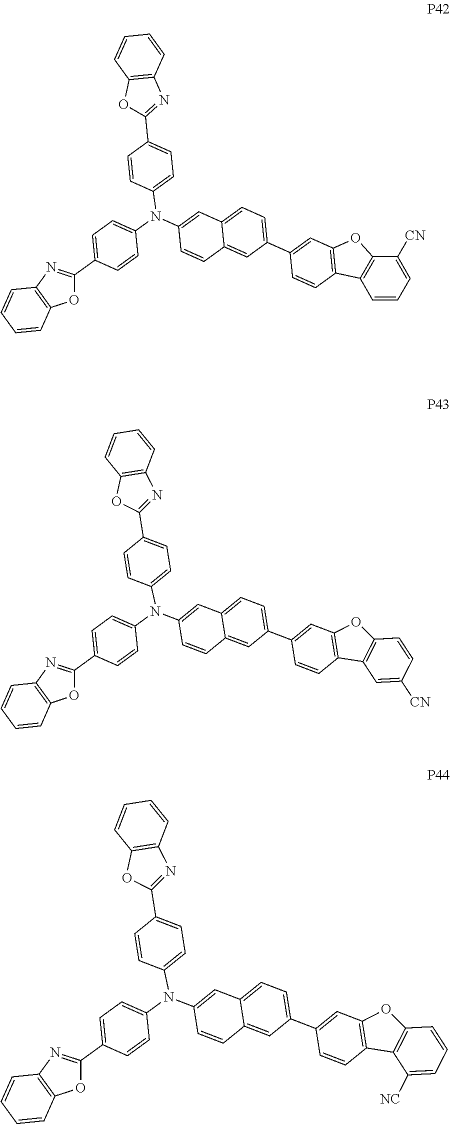 [SMILES: N#Cc1cccc2c1oc1cc(-c3ccc4cc(N(c5ccc(-c6nc7ccccc7o6)cc5)c5ccc(-c6nc7ccccc7o6)cc5)ccc4c3)ccc12 ; N#Cc1ccc2oc3cc(-c4ccc5cc(N(c6ccc(-c7nc8ccccc8o7)cc6)c6ccc(-c7nc8ccccc8o7)cc6)ccc5c4)ccc3c2c1 ; N#Cc1cccc2oc3cc(-c4ccc5cc(N(c6ccc(-c7nc8ccccc8o7)cc6)c6ccc(-c7nc8ccccc8o7)cc6)ccc5c4)ccc3c12]

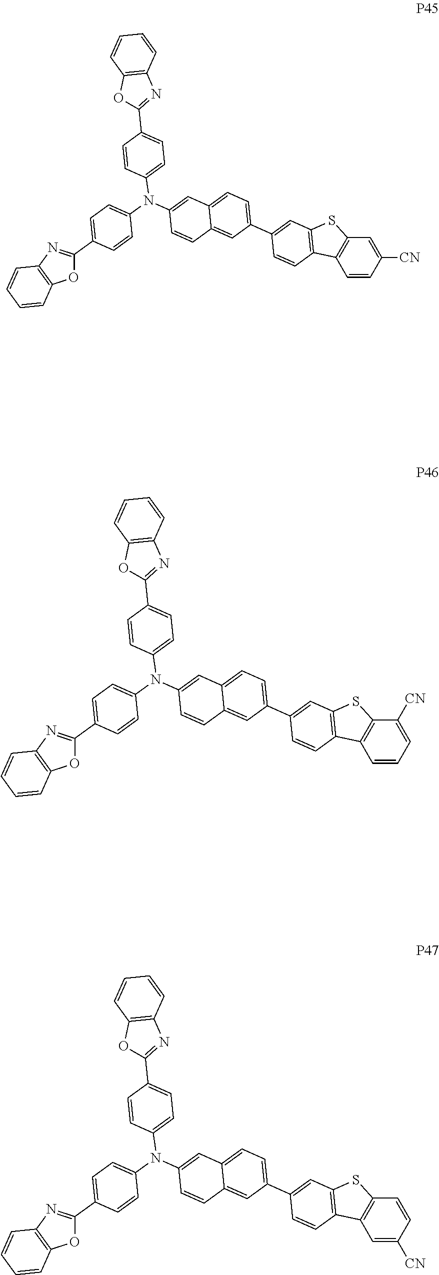 [SMILES: N#Cc1ccc2c(c1)sc1cc(-c3ccc4cc(N(c5ccc(-c6nc7ccccc7o6)cc5)c5ccc(-c6nc7ccccc7o6)cc5)ccc4c3)ccc12 ; N#Cc1cccc2c1sc1cc(-c3ccc4cc(N(c5ccc(-c6nc7ccccc7o6)cc5)c5ccc(-c6nc7ccccc7o6)cc5)ccc4c3)ccc12 ; N#Cc1ccc2sc3cc(-c4ccc5cc(N(c6ccc(-c7nc8ccccc8o7)cc6)c6ccc(-c7nc8ccccc8o7)cc6)ccc5c4)ccc3c2c1]

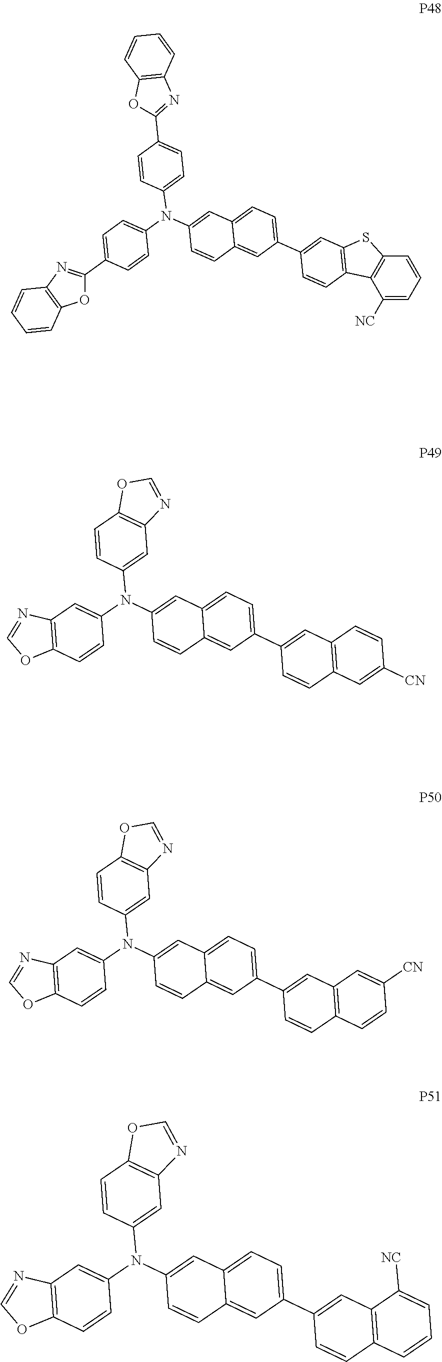 [SMILES: N#Cc1cccc2sc3cc(-c4ccc5cc(N(c6ccc(-c7nc8ccccc8o7)cc6)c6ccc(-c7nc8ccccc8o7)cc6)ccc5c4)ccc3c12 ; N#Cc1ccc2cc(-c3ccc4cc(N(c5ccc6ocnc6c5)c5ccc6ocnc6c5)ccc4c3)ccc2c1 ; N#Cc1ccc2ccc(-c3ccc4cc(N(c5ccc6ocnc6c5)c5ccc6ocnc6c5)ccc4c3)cc2c1 ; N#Cc1cccc2ccc(-c3ccc4cc(N(c5ccc6ocnc6c5)c5ccc6ocnc6c5)ccc4c3)cc12]

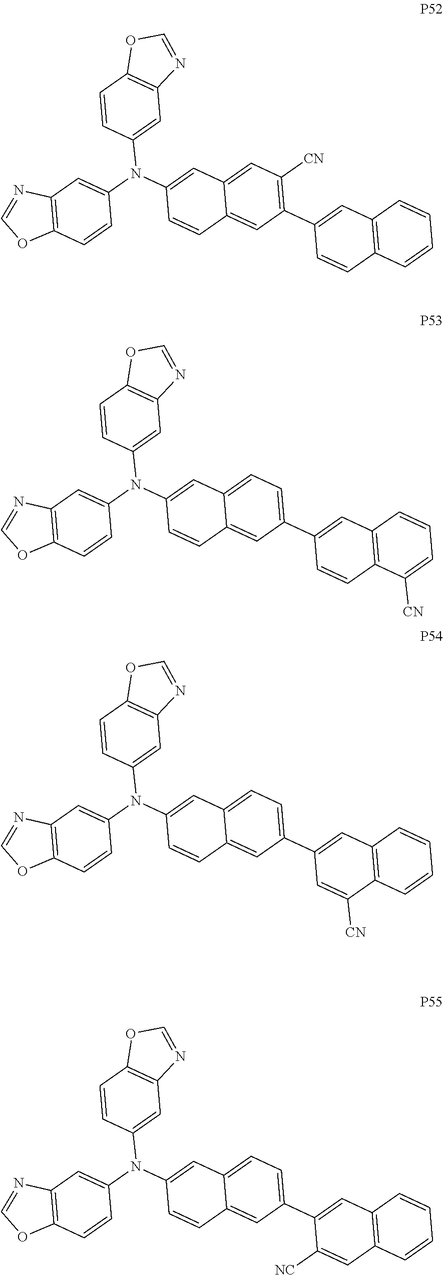 [SMILES: N#Cc1cc2cc(N(c3ccc4ocnc4c3)c3ccc4ocnc4c3)ccc2cc1-c1ccc2ccccc2c1 ; N#Cc1cccc2cc(-c3ccc4cc(N(c5ccc6ocnc6c5)c5ccc6ocnc6c5)ccc4c3)ccc12 ; N#Cc1cc(-c2ccc3cc(N(c4ccc5ocnc5c4)c4ccc5ocnc5c4)ccc3c2)cc2ccccc12 ; N#Cc1cc2ccccc2cc1-c1ccc2cc(N(c3ccc4ocnc4c3)c3ccc4ocnc4c3)ccc2c1]

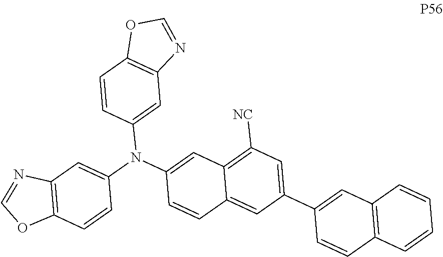 [SMILES: N#Cc1cc(-c2ccc3ccccc3c2)cc2ccc(N(c3ccc4ocnc4c3)c3ccc4ocnc4c3)cc12]

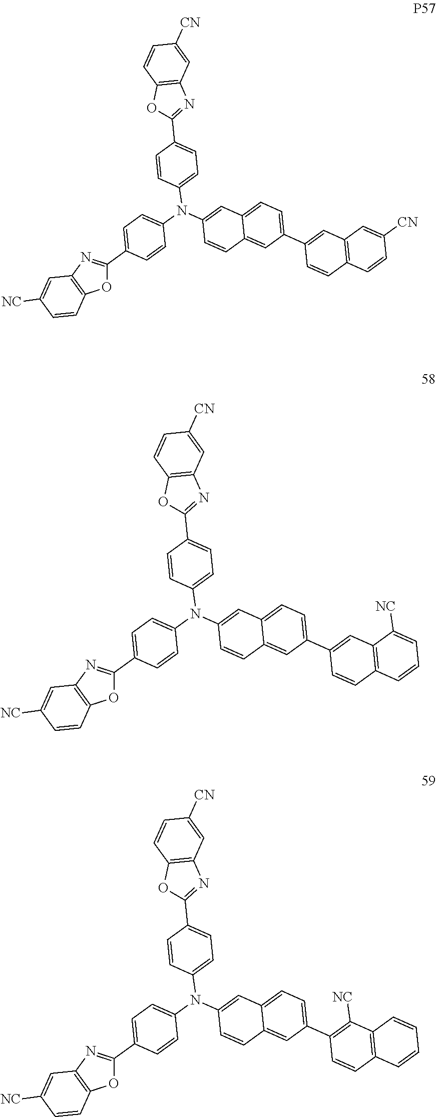 [SMILES: N#Cc1ccc2ccc(-c3ccc4cc(N(c5ccc(-c6nc7cc(C#N)ccc7o6)cc5)c5ccc(-c6nc7cc(C#N)ccc7o6)cc5)ccc4c3)cc2c1 ; N#Cc1ccc2oc(-c3ccc(N(c4ccc(-c5nc6cc(C#N)ccc6o5)cc4)c4ccc5cc(-c6ccc7cccc(C#N)c7c6)ccc5c4)cc3)nc2c1 ; N#Cc1ccc2oc(-c3ccc(N(c4ccc(-c5nc6cc(C#N)ccc6o5)cc4)c4ccc5cc(-c6ccc7ccccc7c6C#N)ccc5c4)cc3)nc2c1]

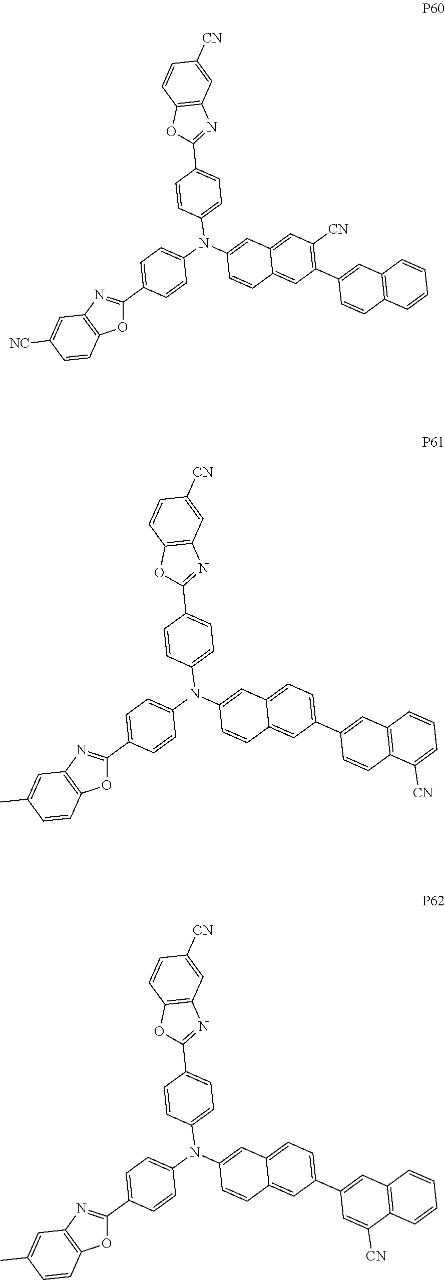 [SMILES: N#Cc1ccc2oc(-c3ccc(N(c4ccc(-c5nc6cc(C#N)ccc6o5)cc4)c4ccc5cc(-c6ccc7ccccc7c6)c(C#N)cc5c4)cc3)nc2c1 ; Cc1ccc2oc(-c3ccc(N(c4ccc(-c5nc6cc(C#N)ccc6o5)cc4)c4ccc5cc(-c6ccc7c(C#N)cccc7c6)ccc5c4)cc3)nc2c1 ; Cc1ccc2oc(-c3ccc(N(c4ccc(-c5nc6cc(C#N)ccc6o5)cc4)c4ccc5cc(-c6cc(C#N)c7ccccc7c6)ccc5c4)cc3)nc2c1]

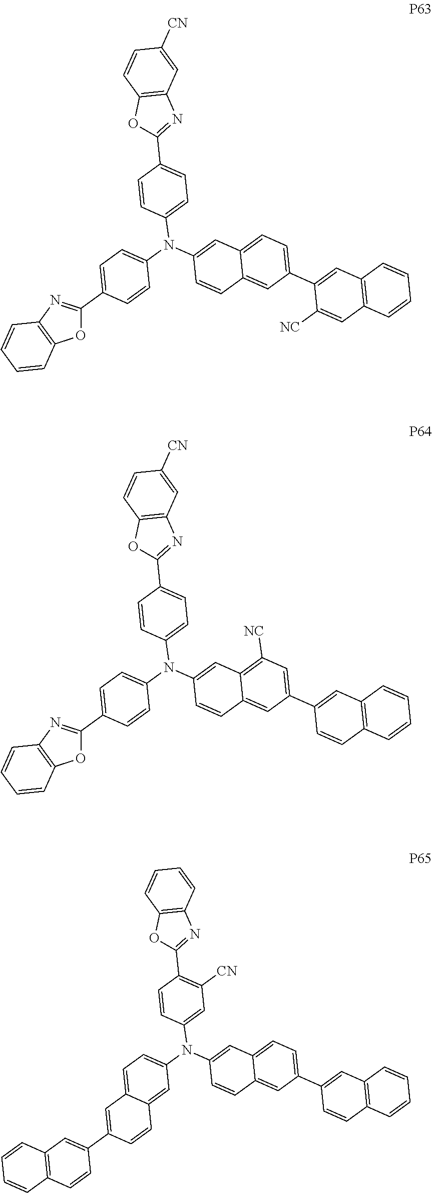 [SMILES: N#Cc1ccc2oc(-c3ccc(N(c4ccc(-c5nc6ccccc6o5)cc4)c4ccc5cc(-c6cc7ccccc7cc6C#N)ccc5c4)cc3)nc2c1 ; N#Cc1ccc2oc(-c3ccc(N(c4ccc(-c5nc6ccccc6o5)cc4)c4ccc5cc(-c6ccc7ccccc7c6)cc(C#N)c5c4)cc3)nc2c1 ; N#Cc1cc(N(c2ccc3cc(-c4ccc5ccccc5c4)ccc3c2)c2ccc3cc(-c4ccc5ccccc5c4)ccc3c2)ccc1-c1nc2ccccc2o1]

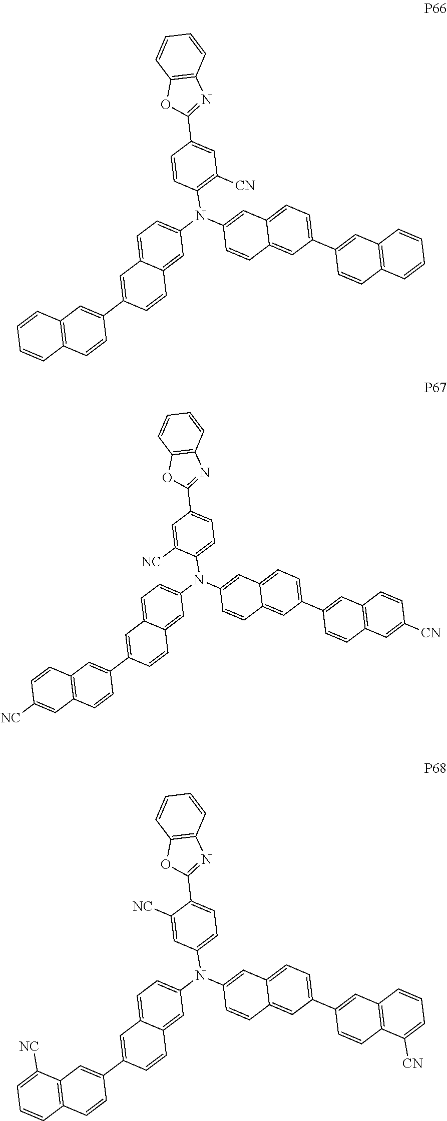 [SMILES: N#Cc1cc(-c2nc3ccccc3o2)ccc1N(c1ccc2cc(-c3ccc4ccccc4c3)ccc2c1)c1ccc2cc(-c3ccc4ccccc4c3)ccc2c1 ; N#Cc1ccc2cc(-c3ccc4cc(N(c5ccc6cc(-c7ccc8cc(C#N)ccc8c7)ccc6c5)c5ccc(-c6nc7ccccc7o6)cc5C#N)ccc4c3)ccc2c1 ; N#Cc1cc(N(c2ccc3cc(-c4ccc5c(C#N)cccc5c4)ccc3c2)c2ccc3cc(-c4ccc5cccc(C#N)c5c4)ccc3c2)ccc1-c1nc2ccccc2o1]

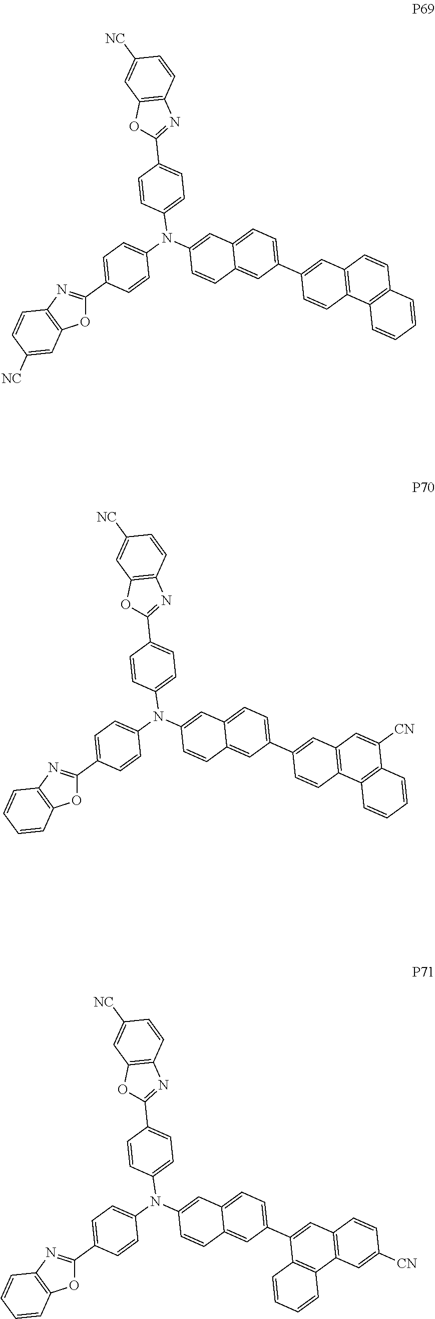 [SMILES: N#Cc1ccc2nc(-c3ccc(N(c4ccc(-c5nc6ccc(C#N)cc6o5)cc4)c4ccc5cc(-c6ccc7c(ccc8ccccc87)c6)ccc5c4)cc3)oc2c1 ; N#Cc1ccc2nc(-c3ccc(N(c4ccc(-c5nc6ccccc6o5)cc4)c4ccc5cc(-c6ccc7c(c6)cc(C#N)c6ccccc67)ccc5c4)cc3)oc2c1 ; N#Cc1ccc2nc(-c3ccc(N(c4ccc(-c5nc6ccccc6o5)cc4)c4ccc5cc(-c6cc7ccc(C#N)cc7c7ccccc67)ccc5c4)cc3)oc2c1]

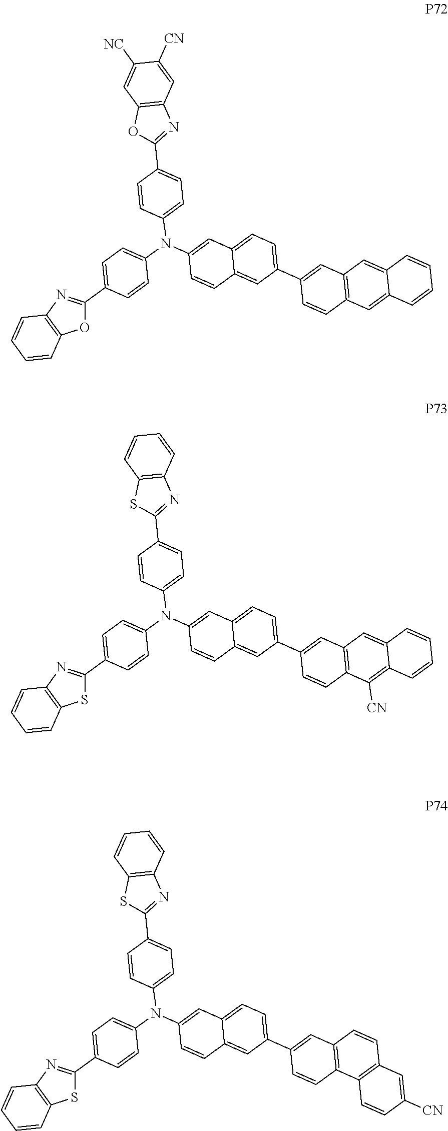 [SMILES: N#Cc1cc2nc(-c3ccc(N(c4ccc(-c5nc6ccccc6o5)cc4)c4ccc5cc(-c6ccc7cc8ccccc8cc7c6)ccc5c4)cc3)oc2cc1C#N ; N#Cc1c2ccccc2cc2cc(-c3ccc4cc(N(c5ccc(-c6nc7ccccc7s6)cc5)c5ccc(-c6nc7ccccc7s6)cc5)ccc4c3)ccc12 ; N#Cc1ccc2c(ccc3cc(-c4ccc5cc(N(c6ccc(-c7nc8ccccc8s7)cc6)c6ccc(-c7nc8ccccc8s7)cc6)ccc5c4)ccc32)c1]

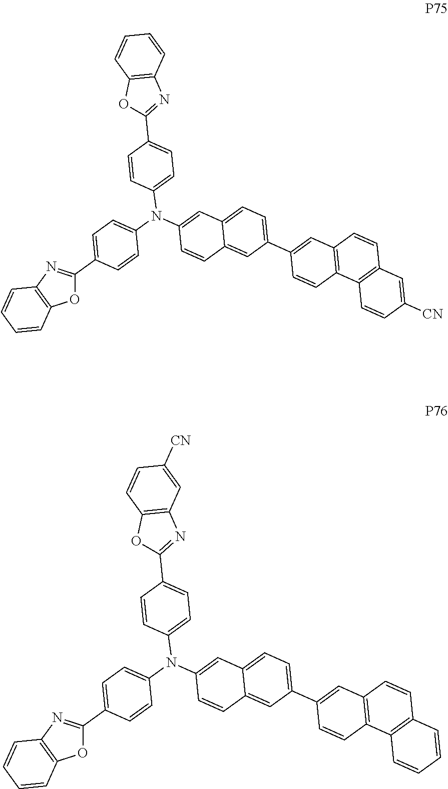 [SMILES: N#Cc1ccc2c(ccc3cc(-c4ccc5cc(N(c6ccc(-c7nc8ccccc8o7)cc6)c6ccc(-c7nc8ccccc8o7)cc6)ccc5c4)ccc32)c1 ; N#Cc1ccc2oc(-c3ccc(N(c4ccc(-c5nc6ccccc6o5)cc4)c4ccc5cc(-c6ccc7c(ccc8ccccc87)c6)ccc5c4)cc3)nc2c1]

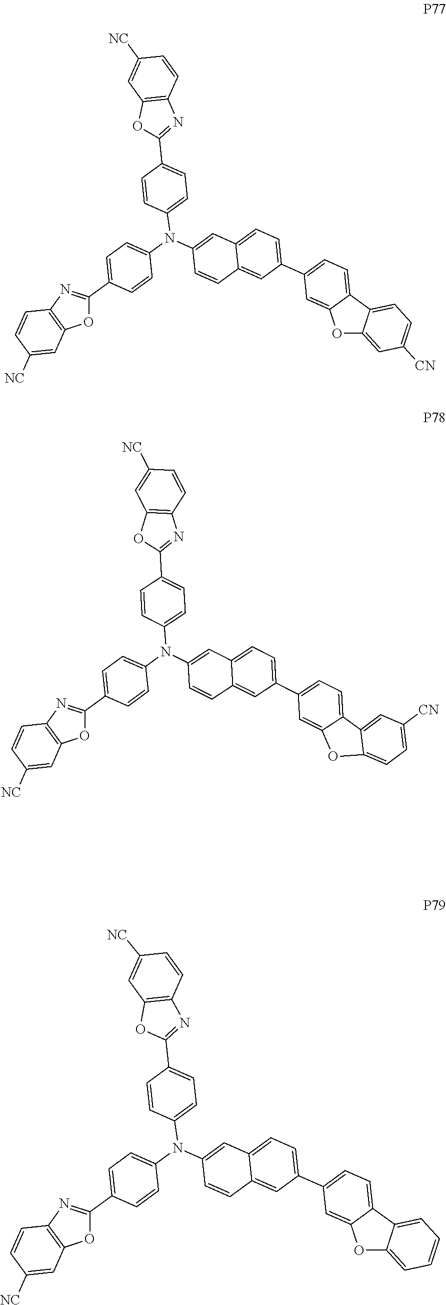

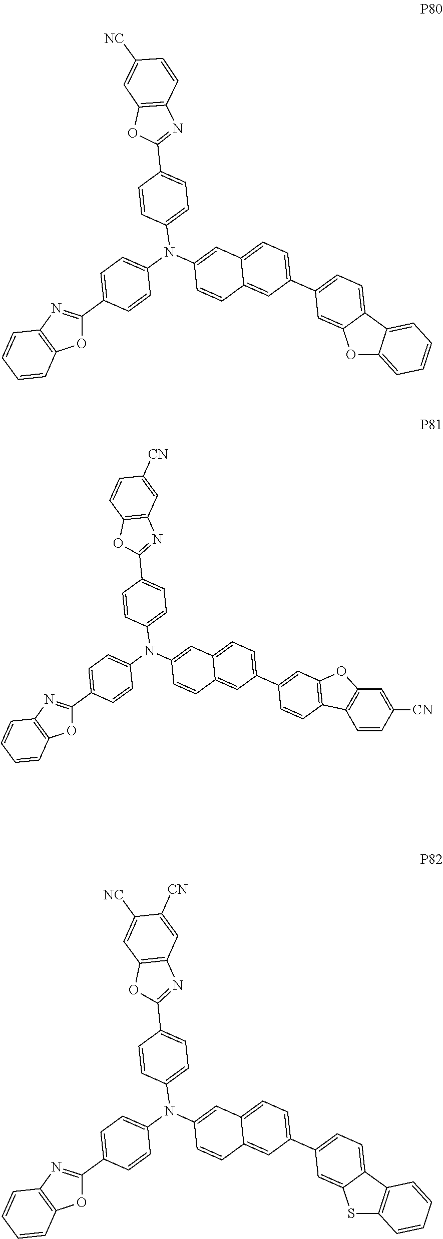

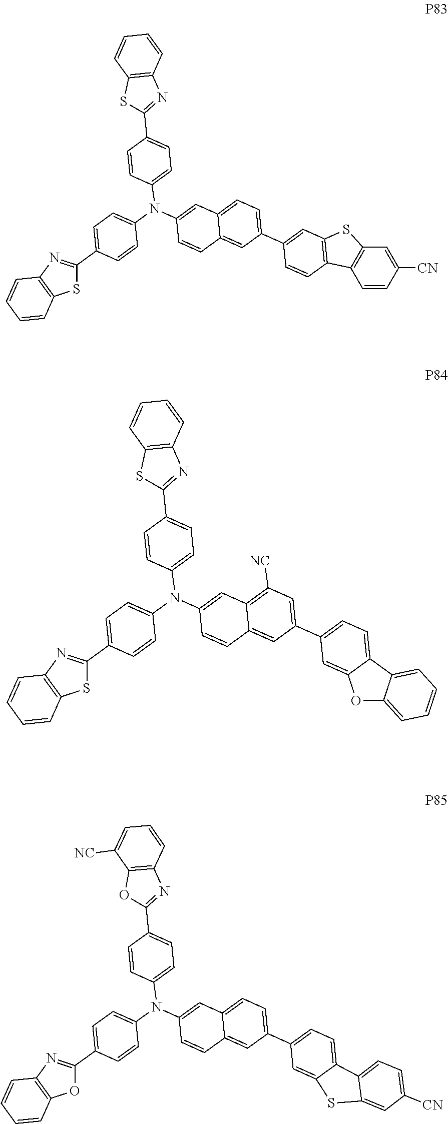

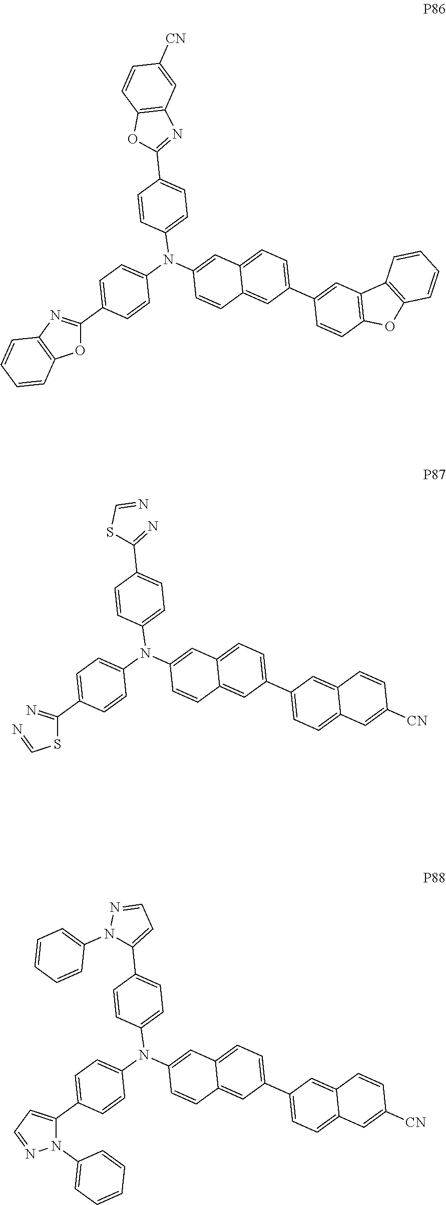

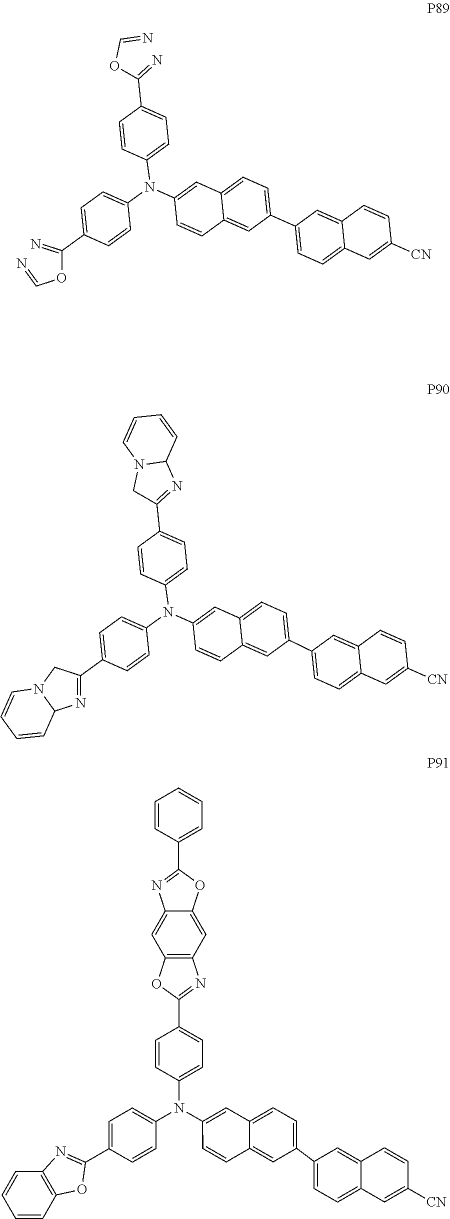 [SMILES: N#Cc1ccc2cc(-c3ccc4cc(N(c5ccc(C(=N)OC=N)cc5)c5ccc(C(=N)OC=N)cc5)ccc4c3)ccc2c1 ; N#Cc1ccc2cc(-c3ccc4cc(N(c5ccc(C6=NC7C=CC=CN7C6)cc5)c5ccc(C6=NC7C=CC=CN7C6)cc5)ccc4c3)ccc2c1 ; N#Cc1ccc2cc(-c3ccc4cc(N(c5ccc(-c6nc7ccccc7o6)cc5)c5ccc(-c6nc7cc8oc(-c9ccccc9)nc8cc7o6)cc5)ccc4c3)ccc2c1]

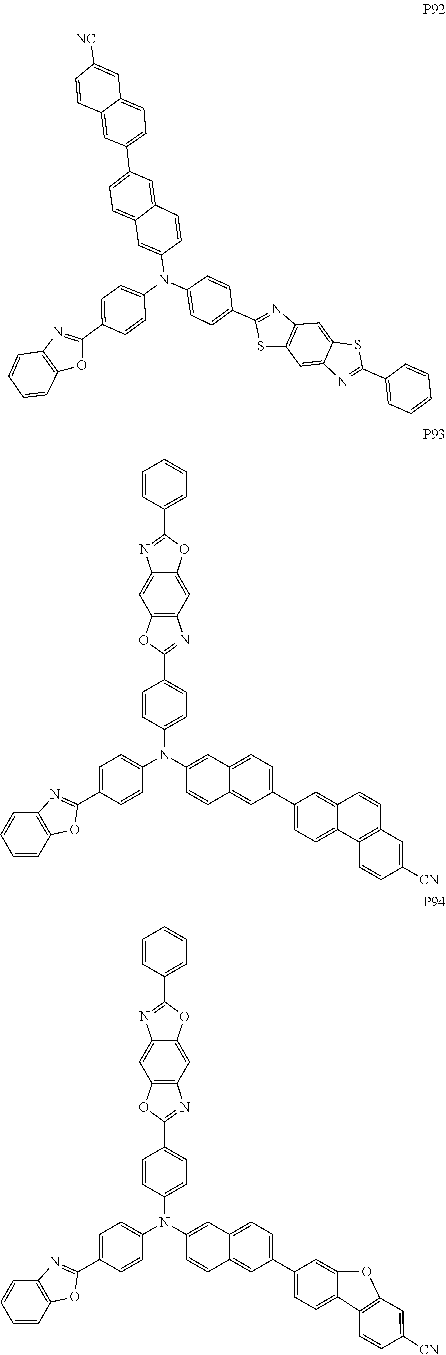 [SMILES: N#Cc1ccc2cc(-c3ccc4cc(N(c5ccc(-c6nc7ccccc7o6)cc5)c5ccc(-c6nc7cc8sc(-c9ccccc9)nc8cc7s6)cc5)ccc4c3)ccc2c1 ; N#Cc1ccc2c(ccc3cc(-c4ccc5cc(N(c6ccc(-c7nc8ccccc8o7)cc6)c6ccc(-c7nc8cc9oc(-c%10ccccc%10)nc9cc8o7)cc6)ccc5c4)ccc32)c1 ; N#Cc1ccc2c(c1)oc1cc(-c3ccc4cc(N(c5ccc(-c6nc7ccccc7o6)cc5)c5ccc(-c6nc7cc8oc(-c9ccccc9)nc8cc7o6)cc5)ccc4c3)ccc12]

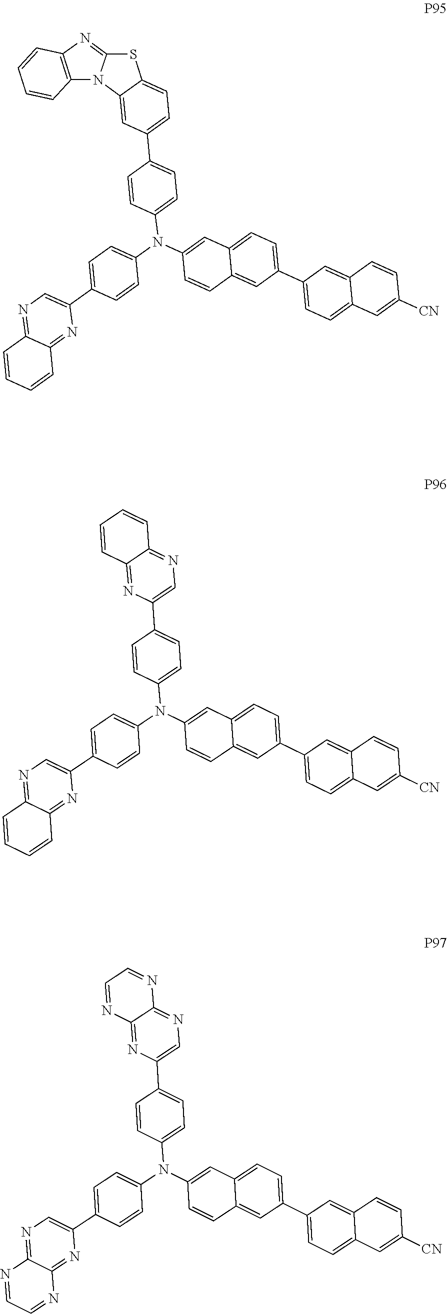 [SMILES: N#Cc1ccc2cc(-c3ccc4cc(N(c5ccc(-c6ccc7sc8nc9ccccc9n8c7c6)cc5)c5ccc(-c6cnc7ccccc7n6)cc5)ccc4c3)ccc2c1 ; N#Cc1ccc2cc(-c3ccc4cc(N(c5ccc(-c6cnc7ccccc7n6)cc5)c5ccc(-c6cnc7ccccc7n6)cc5)ccc4c3)ccc2c1 ; N#Cc1ccc2cc(-c3ccc4cc(N(c5ccc(-c6cnc7nccnc7n6)cc5)c5ccc(-c6cnc7nccnc7n6)cc5)ccc4c3)ccc2c1]

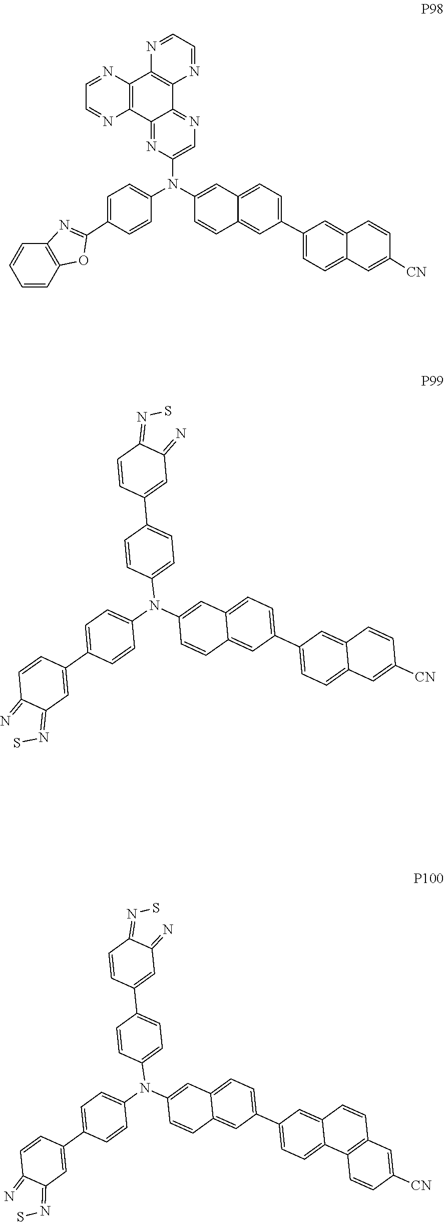 [SMILES: N#Cc1ccc2cc(-c3ccc4cc(N(c5ccc(-c6nc7ccccc7o6)cc5)c5cnc6c7nccnc7c7nccnc7c6n5)ccc4c3)ccc2c1 ; N#Cc1ccc2cc(-c3ccc4cc(N(c5ccc(C6=CC(=N)/C(=N\S)C=C6)cc5)c5ccc(C6=C/C(=N/S)C(=N)C=C6)cc5)ccc4c3)ccc2c1 ; N#Cc1ccc2c(ccc3cc(-c4ccc5cc(N(c6ccc(C7=CC(=N)/C(=N\S)C=C7)cc6)c6ccc(C7=C/C(=N/S)C(=N)C=C7)cc6)ccc5c4)ccc32)c1]

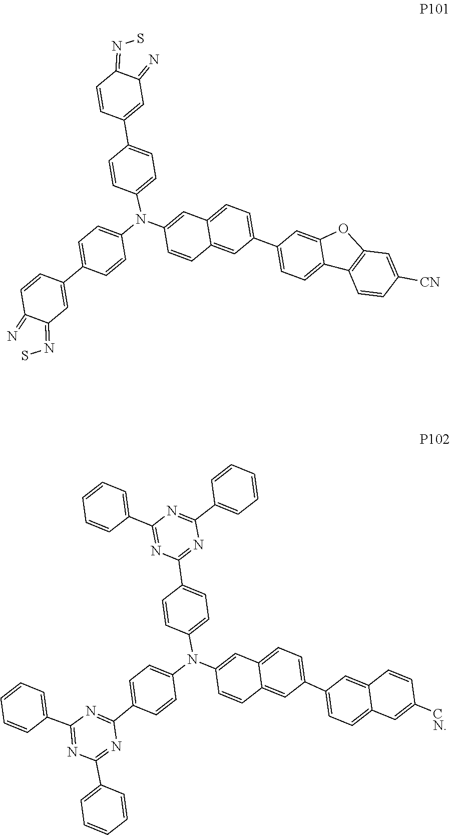 [SMILES: N#Cc1ccc2c(c1)oc1cc(-c3ccc4cc(N(c5ccc(C6=CC(=N)/C(=N\S)C=C6)cc5)c5ccc(C6=C/C(=N/S)C(=N)C=C6)cc5)ccc4c3)ccc12 ; N#Cc1ccc2cc(-c3ccc4cc(N(c5ccc(-c6nc(-c7ccccc7)nc(-c7ccccc7)n6)cc5)c5ccc(-c6nc(-c7ccccc7)nc(-c7ccccc7)n6)cc5)ccc4c3)ccc2c1]

A second object of the present disclosure is to provide a material for an organic electroluminescent device. The material for the organic electroluminescent device includes any one or a combination of at least two of the compounds as described for the first object.

A third object of the present disclosure is to provide an organic electroluminescent device. The organic electroluminescent device includes a first electrode layer, an organic function layer and a second electrode layer which are stacked in sequence;

wherein the organic function layer includes the material as described for the second object.

A fourth object of the present disclosure is to provide an organic electroluminescent device. The organic electroluminescent device includes a first capping layer, a first electrode layer, an organic function layer and a second electrode layer which are stacked in sequence;

wherein the first capping layer includes the material as described for the second object.

When the device is a top emitting device, the first electrode layer is a cathode layer and the second electrode layer is an anode layer; when the device is a bottom emitting device, the first electrode layer is the anode layer and the second electrode layer is the cathode layer.

The compound of the present disclosure can interact with a metal in a cathode (or anode) of the device, which reduces a coupling effect between free charges on the surface of the metal and electromagnetic radiation and improves photon extraction efficiency. Meanwhile, this modifies a metal electrode and reduces the possibility of film peeling.

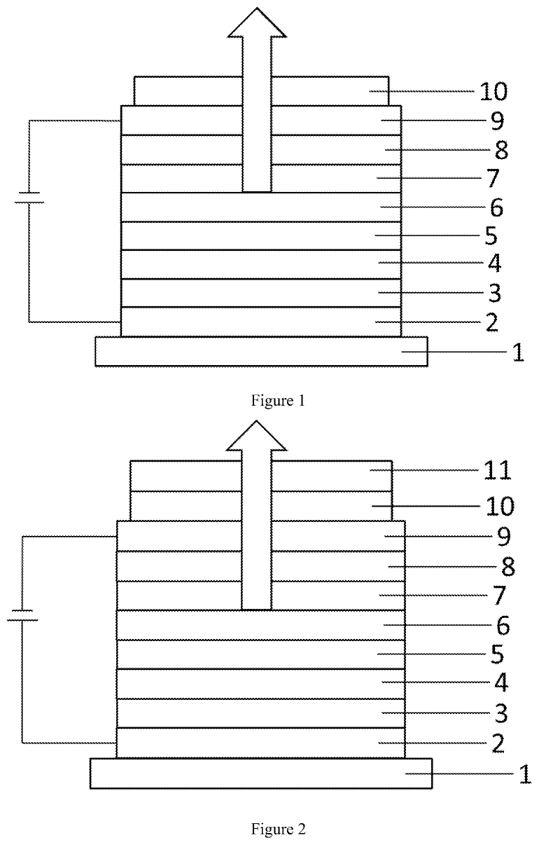

In an embodiment, an organic optoelectronic device provided by the present disclosure, as shown in FIG. 1, includes a substrate 1, an anode 2, a hole injection layer 3, a first hole transport layer 4, a second hole transport layer 5, a light-emitting layer 6, a first electron transport layer 7, a second electron transport layer 8, a cathode 9 and a first capping layer 10.

In an embodiment, the organic electroluminescent device further includes a second capping layer disposed on a side of the first capping layer facing away from the first electrode layer, where the second capping layer includes a material containing lithium fluoride and/or a small organic molecule with a refractive index of 1.40-1.65 (for example, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64 or the like).

The organic electroluminescent device provided by the present disclosure preferably includes two capping layers, and the compound provided by the present disclosure cooperates with lithium fluoride and/or the small organic molecule with a refractive index of 1.40-1.65, which can alleviate the total reflection of light by a packaging glass, facilitate the transmission of visible light through the glass and improve a light extraction effect.

In an embodiment, an organic optoelectronic device provided by the present disclosure, as shown in FIG. 2, includes a substrate 1, an anode 2, a hole injection layer 3, a first hole transport layer 4, a second hole transport layer 5, a light-emitting layer 6, a first electron transport layer 7, a second electron transport layer 8, a cathode 9, a first capping layer 10 and a second capping layer 11.

In an embodiment, a material of the small organic molecule with a refractive index of 1.40-1.65 includes, but is not limited to, any one or a combination of at least two of polyfluorocarbons, boron-containing compounds, silicon-containing compounds, oxygen-containing silicon compounds or adamantane-containing alkane compounds.

A fifth object of the present disclosure is to provide a display panel. The display panel includes the organic electroluminescent device as described for the third object or the fourth object.

In an embodiment, the display panel is a foldable display panel.

When the compound provided by the present disclosure is used in the foldable display panel for display at multiple angles, light extraction Δn is relatively small for RGB colors, which can effectively reduce a color cast.

A sixth object of the present disclosure is to provide a display device. The display device includes the display panel as described for the fifth object.

A method for preparing the compound provided by the present disclosure belongs to the related art, and those skilled in the art can select a specific synthesis method according to conventional technical knowledge. The present disclosure provides only an exemplary synthesis route and the preparing method is not limited to synthesis routes described below.

A representative synthesis route of the compound provided by the present disclosure is as follows:

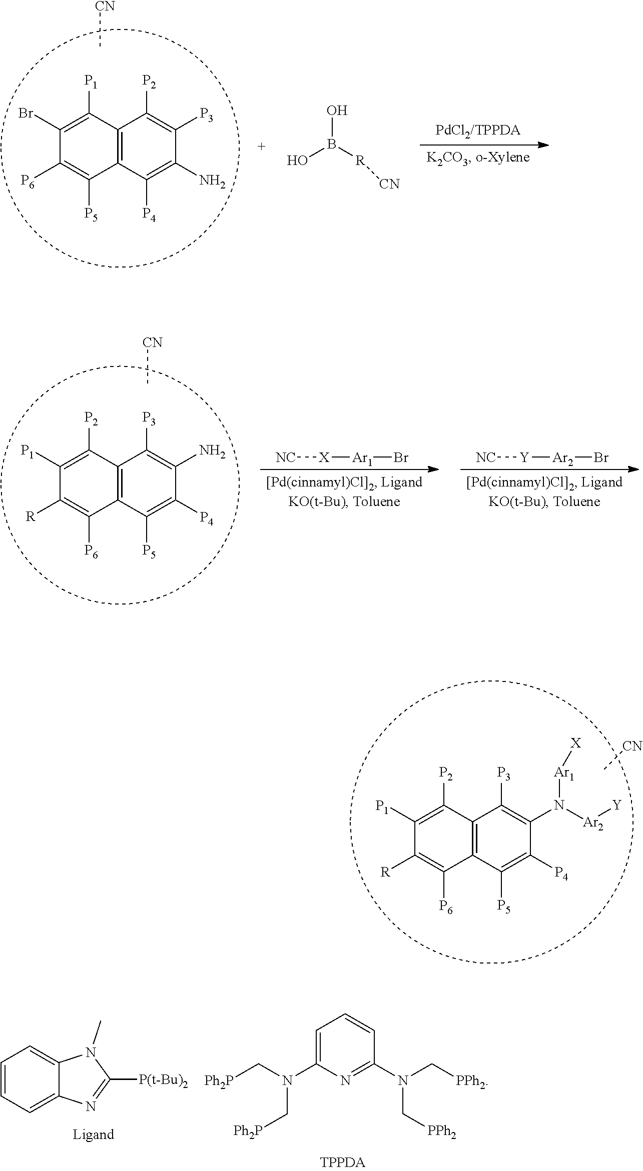

In the above synthesis route, a position where CN may be substitutable is shown by a dashed line, and a dashed circle indicates that CN may be substituted at any substitutable position within the ring.

In the above synthesis route, o-Xylene represents ortho-xylene, Toluene represents toluene, KO(t-Bu) represents potassium tert-butoxide, and [Pd(cinnamyl)C1]₂ represents palladium chloride (1-phenylallyl).

The following examples exemplarily provide synthesis methods for a series of specific compounds. Compounds whose specific synthesis methods are not mentioned may be synthesized by similar methods or other existing methods, which is not specifically limited in the present disclosure.

### Example 1

Synthesis of Compound P1

A specific preparation method specifically includes steps described below.

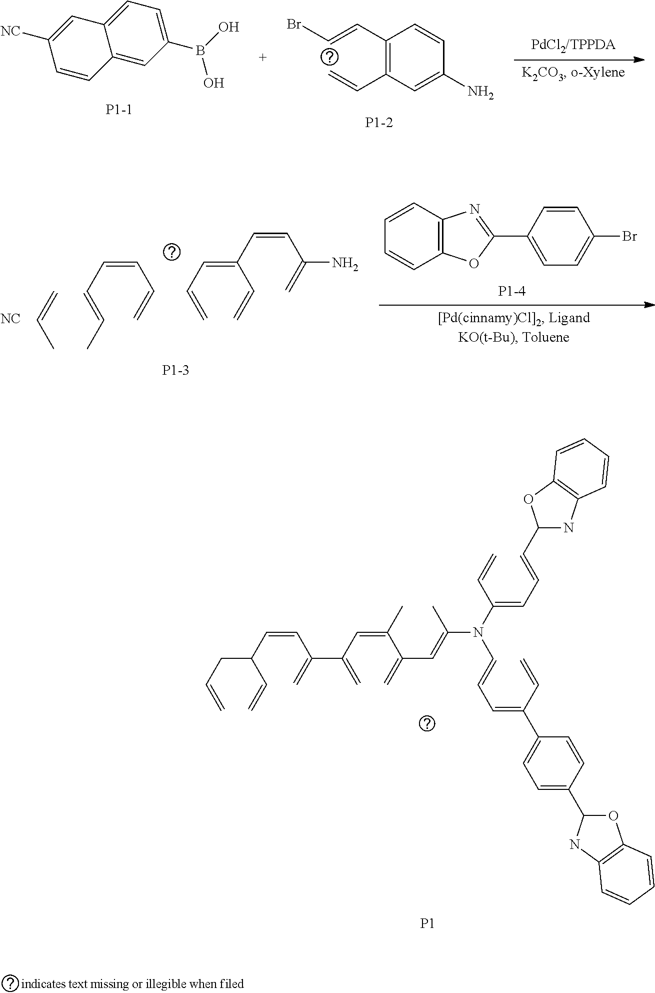

(1) P1-1 (0.5 mmol), P1-2 (0.75 mmol), K₂CO₃ (0.5 mmol), PdCl₂ (5×10⁻⁴ mmol) and TPPDA (5×10⁻⁴ mmol) were added to 3 mL of o-xylene and mixed into a solution, and the solution was put in a 50 mL flask and reacted at 100° C. for 24 h. The solution was cooled to room temperature and slowly added with a saturated aqueous solution of MgSO₄ and ethyl acetate to be extracted three times. Then, the organic layer passed through a rotary evaporator for the solvent to be removed and the crude product P1-3 was obtained through column chromatography.

The structure of the target product P1-3 was tested through matrix-assisted laser desorption/ionization time-of-flight mass spectrometry (MALDI-TOF-MS) (m/z). The structure was obtained as C₂₁H₁₄N₂ whose calculated value was 294.1 and measured value was 294.0.

(2) P1-3 (0.5 mmol), P1-4 (1.5 mmol), KO(t-Bu) (0.75 mmol), [Pd(cinnamyl)C1]₂ (0.02 mol) and a ligand (0.015 mol) were added to 3 mL of toluene and mixed into a solution, and the solution was put in a 50 mL flask and reacted at 80° C. for 12 h. The solution was cooled to room temperature and slowly added with a saturated aqueous solution of MgSO₄ and ethyl acetate to be extracted three times. Then, the organic layer passed through a rotary evaporator for the solvent to be removed and the crude product P1 was obtained through column chromatography.

The structure of the target product P1 was tested through matrix-assisted laser desorption/ionization time-of-flight mass spectrometry (MALDI-TOF-MS) (m/z). The structure was obtained as C₄₇H₂₈N₄O₂ whose calculated value was 680.2 and measured value was 680.1.

Elemental analysis: theoretical value: C, 82.92; H, 4.15; N, 8.23; measured value: C, 82.91; H, 4.15; N, 8.23.

### Example 2

Synthesis of Compound P8

A specific preparation method specifically includes steps described below.

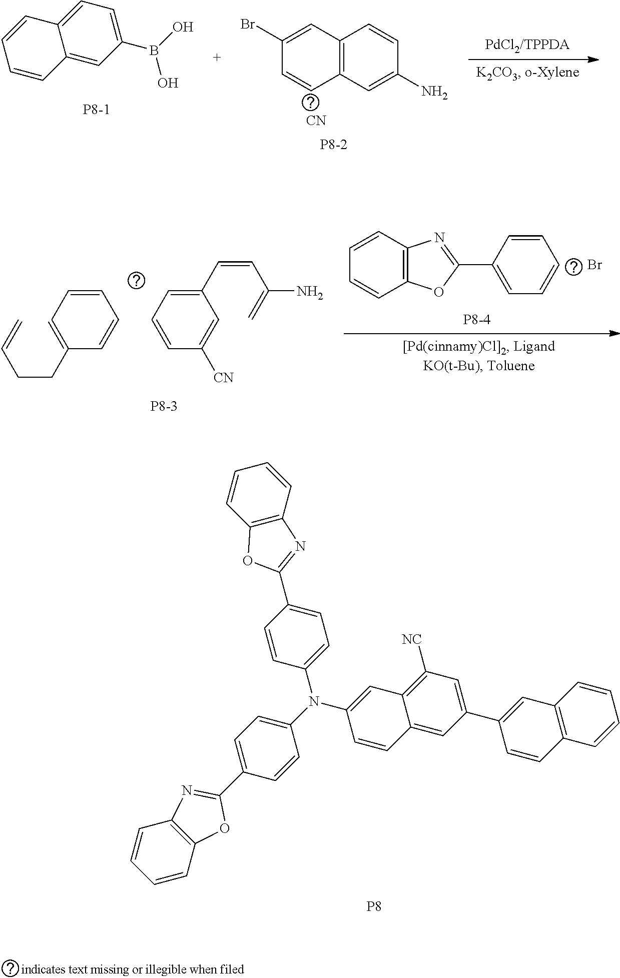

(1) P8-1 (0.5 mmol), P8-2 (0.75 mmol), K₂CO₃ (0.5 mmol), PdCl₂ (5×10⁻⁴ mmol) and TPPDA (5×10⁴ mmol) were added to 3 mL of o-xylene and mixed into a solution, and the solution was put in a 50 mL flask and reacted at 100° C. for 24 h. The solution was cooled to room temperature and slowly added with a saturated aqueous solution of MgSO₄ and ethyl acetate to be extracted three times. Then, the organic layer passed through a rotary evaporator for the solvent to be removed and the crude product P8-3 was obtained through column chromatography.

The structure of the target product P8-3 was tested through matrix-assisted laser desorption/ionization time-of-flight mass spectrometry (MALDI-TOF-MS) (m/z). The structure was obtained as C₂₁H₁₄N₂ whose calculated value was 294.1 and measured value was 294.1.

(2) P8-3 (0.5 mmol), P8-4 (1.5 mmol), KO(t-Bu) (0.75 mmol), [Pd(cinnamyl)C1]₂ (0.02 mol) and a ligand (0.015 mol) were added to 3 mL of toluene and mixed into a solution, and the solution was put in a 50 mL flask and reacted at 80° C. for 12 h. The solution was cooled to room temperature and slowly added with a saturated aqueous solution of MgSO₄ and ethyl acetate to be extracted three times. Then, the organic layer passed through a rotary evaporator for the solvent to be removed and the crude product P8 was obtained through column chromatography.

The structure of the target product P8 was tested through matrix-assisted laser desorption/ionization time-of-flight mass spectrometry (MALDI-TOF-MS) (m/z). The structure was obtained as C₄₇H₂₈N₄O₂ whose calculated value was 680.2 and measured value was 680.1.

Elemental analysis: theoretical value: C, 82.92; H, 4.15; N, 8.23; measured value: C, 82.92; H, 4.16; N, 8.22.

### Example 3

Synthesis of Compound P11

A specific preparation method specifically includes a step described below.

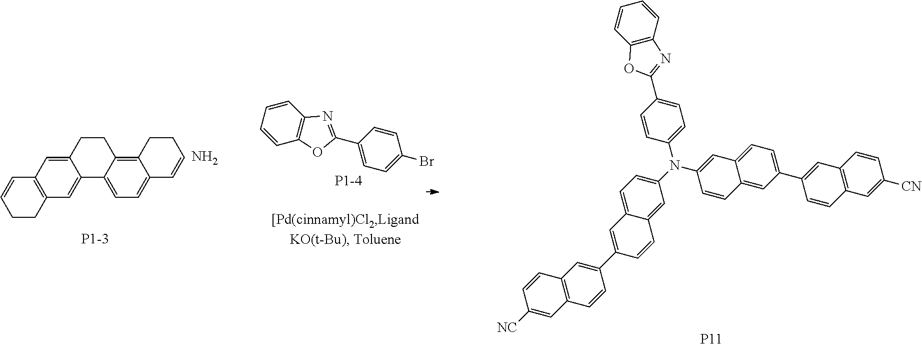

(1) P1-3 (1.5 mmol), P1-4 (0.5 mmol), KO(t-Bu) (0.75 mmol), [Pd(cinnamyl)C1]₂ (0.02 mol) and a ligand (0.015 mol) were added to 3 mL of toluene and mixed into a solution, and the solution was put in a 50 mL flask and reacted at 80° C. for 12 h. The solution was cooled to room temperature and slowly added with a saturated aqueous solution of MgSO₄ and ethyl acetate to be extracted three times. Then, the organic layer passed through a rotary evaporator for the solvent to be removed and the crude product P11 was obtained through column chromatography.

The structure of the target product P11 was tested through matrix-assisted laser desorption/ionization time-of-flight mass spectrometry (MALDI-TOF-MS) (m/z). The structure was obtained as C₅₅H₃₂N₄O whose calculated value was 764.3 and measured value was 764.2.

Elemental analysis: theoretical value: C, 86.37; H, 4.22; N, 7.33; measured value: C, 86.37; H, 4.21; N, 7.33.

### Example 4

Synthesis of Compound P15

A specific preparation method specifically includes steps described below.

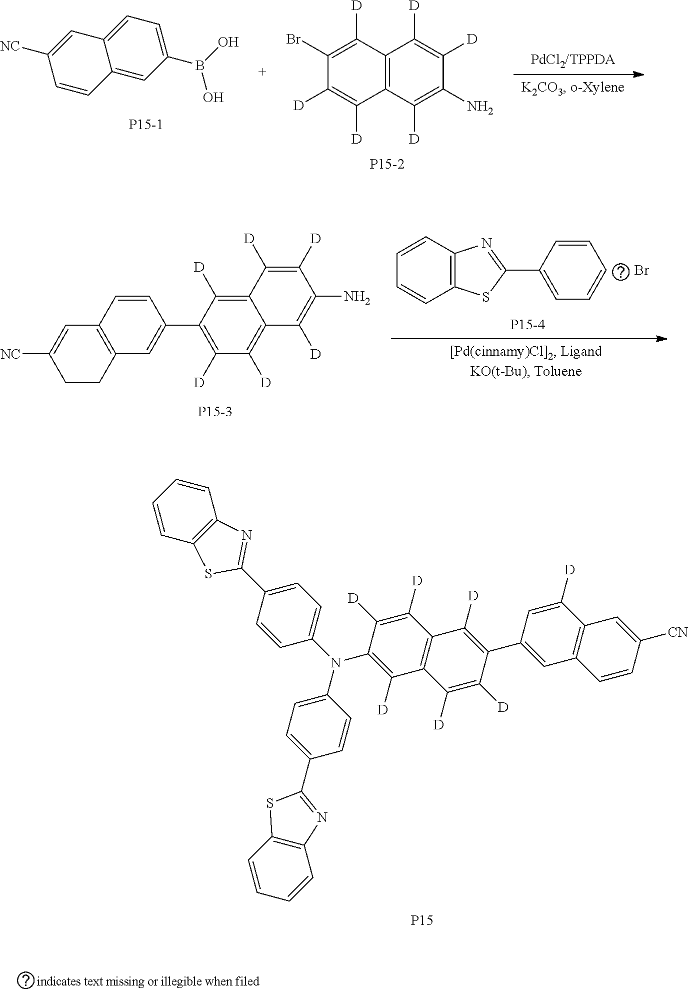

(1) P15-1 (0.5 mmol), P15-2 (0.75 mmol), K₂CO₃ (0.65 mmol), PdCl₂ (6×10⁻⁴ mmol) and TPPDA (6×10⁴ mmol) were added to 3 mL of o-xylene and mixed into a solution, and the solution was put in a 50 mL flask and reacted at 100° C. for 24 h. The solution was cooled to room temperature and slowly added with a saturated aqueous solution of MgSO₄ and ethyl acetate to be extracted three times. Then, the organic layer passed through a rotary evaporator for the solvent to be removed and the crude product P15-3 was obtained through column chromatography.

The structure of the target product P15-3 was tested through matrix-assisted laser desorption/ionization time-of-flight mass spectrometry (MALDI-TOF-MS) (m/z). The structure was obtained as C₂₁H₈D₆N₂ whose calculated value was 300.2 and measured value was 300.0.

(2) P15-3 (0.5 mmol), P15-4 (1.5 mmol), KO(t-Bu) (0.85 mmol), [Pd(cinnamyl)C1]₂ (0.02 mol) and a ligand (0.015 mol) were added to 3 mL of toluene and mixed into a solution, and the solution was put in a 50 mL flask and reacted at 80° C. for 12 h. The solution was cooled to room temperature and slowly added with a saturated aqueous solution of MgSO₄ and ethyl acetate to be extracted three times. Then, the organic layer passed through a rotary evaporator for the solvent to be removed and the crude product P15 was obtained through column chromatography.

The structure of the target product P15 was tested through matrix-assisted laser desorption/ionization time-of-flight mass spectrometry (MALDI-TOF-MS) (m/z). The structure was obtained as C₄₇H₂₂D₆N₄S₂ whose calculated value was 718.2 and measured value was 718.1.

Elemental analysis: theoretical value: C, 78.52; H, 4.77; N, 7.79; measured value: C, 78.53; H, 4.78; N, 7.79.

### Example 5

Synthesis of Compound P29

A specific preparation method specifically includes steps described below.

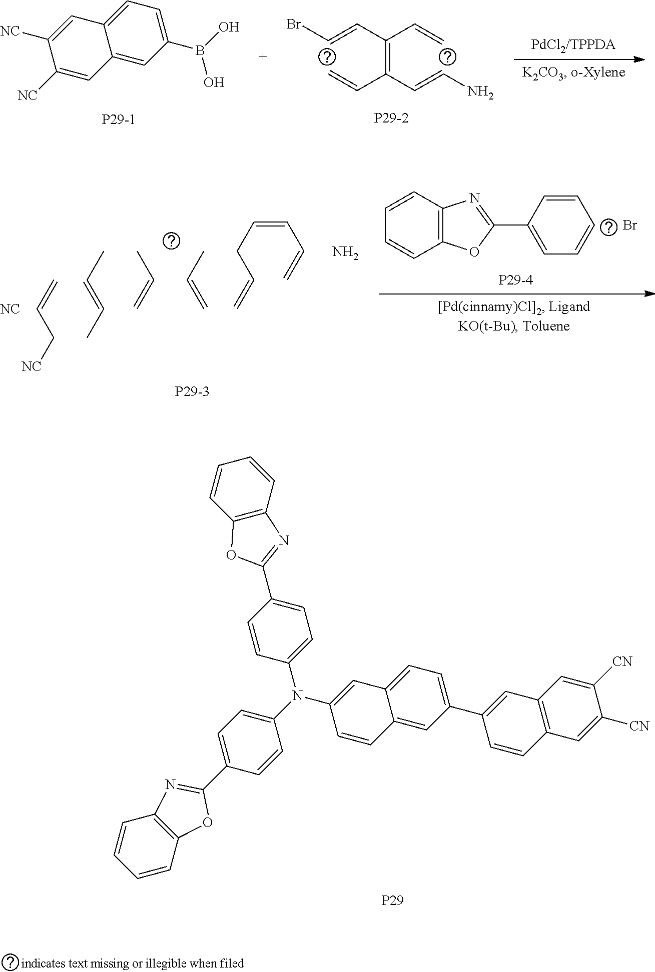

(1) P29-1 (0.5 mmol), P29-2 (0.95 mmol), K₂CO₃ (0.6 mmol), PdCl₂ (5×10⁴ mmol) and TPPDA (5×10⁴ mmol) were added to 3 mL of o-xylene and mixed into a solution, and the solution was put in a 50 mL flask and reacted at 100° C. for 24 h. The solution was cooled to room temperature and slowly added with a saturated aqueous solution of MgSO₄ and ethyl acetate to be extracted three times. Then, the organic layer passed through a rotary evaporator for the solvent to be removed and the crude product P29-3 was obtained through column chromatography.

The structure of the target product P29-3 was tested through matrix-assisted laser desorption/ionization time-of-flight mass spectrometry (MALDI-TOF-MS) (m/z). The structure was obtained as C₂₂H₁₃N₃ whose calculated value was 319.1 and measured value was 319.2.

(2) P29-3 (0.5 mmol), P29-4 (1.5 mmol), KO(t-Bu) (0.75 mmol), [Pd(cinnamyl)C1]₂ (0.02 mol) and a ligand (0.015 mol) were added to 3 mL of toluene and mixed into a solution, and the solution was put in a 50 mL flask and reacted at 80° C. for 12 h. The solution was cooled to room temperature and slowly added with a saturated aqueous solution of MgSO₄ and ethyl acetate to be extracted three times. Then, the organic layer passed through a rotary evaporator for the solvent to be removed and the crude product P29 was obtained through column chromatography.

The structure of the target product P29 was tested through matrix-assisted laser desorption/ionization time-of-flight mass spectrometry (MALDI-TOF-MS) (m/z). The structure was obtained as C₄₈H₂₇N₅O₂ whose calculated value was 705.2 and measured value was 705.1.

Elemental analysis: theoretical value: C, 81.69; H, 3.86; N, 9.92; measured value: C, 81.69; H, 3.87; N, 9.92.

### Example 6

Synthesis of Compound P57

A specific preparation method specifically includes steps described below.

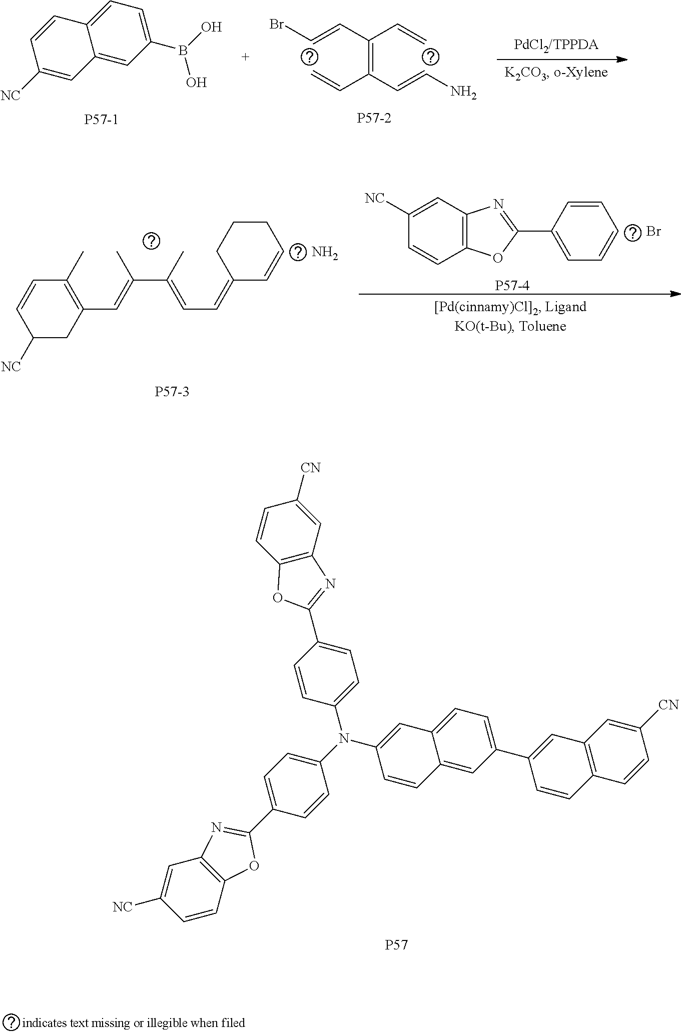

(1) P57-1 (0.6 mmol), P57-2 (0.75 mmol), K₂CO₃ (1.0 mmol), PdCl₂ (6×10⁴ mmol) and TPPDA (6×10⁴ mmol) were added to 3 mL of o-xylene and mixed into a solution, and the solution was put in a 50 mL flask and reacted at 100° C. for 24 h. The solution was cooled to room temperature and slowly added with a saturated aqueous solution of MgSO₄ and ethyl acetate to be extracted three times. Then, the organic layer passed through a rotary evaporator for the solvent to be removed and the crude product P57-3 was obtained through column chromatography.

The structure of the target product P57-3 was tested through matrix-assisted laser desorption/ionization time-of-flight mass spectrometry (MALDI-TOF-MS) (m/z). The structure was obtained as C₂₁H₁₄N₂ whose calculated value was 294.1 and measured value was 294.1.

(2) P57-3 (0.6 mmol), P57-4 (1.5 mmol), KO(t-Bu) (0.85 mmol), [Pd(cinnamyl)C1]₂ (0.025 mol) and a ligand (0.02 mol) were added to 3 mL of toluene and mixed into a solution, and the solution was put in a 50 mL flask and reacted at 80° C. for 12 h. The solution was cooled to room temperature and slowly added with a saturated aqueous solution of MgSO₄ and ethyl acetate to be extracted three times. Then, the organic layer passed through a rotary evaporator for the solvent to be removed and the crude product P57 was obtained through column chromatography.

The structure of the target product P57 was tested through matrix-assisted laser desorption/ionization time-of-flight mass spectrometry (MALDI-TOF-MS) (m/z). The structure was obtained as C₄₉H₂₆N₆O₂ whose calculated value was 730.2 and measured value was 730.1.

Elemental analysis: theoretical value: C, 80.53; H, 3.59; N, 11.50; measured value: C, 80.53; H, 3.60; N, 11.51.

### Example 7

Synthesis of Compound P65

A specific preparation method specifically includes a step described below.

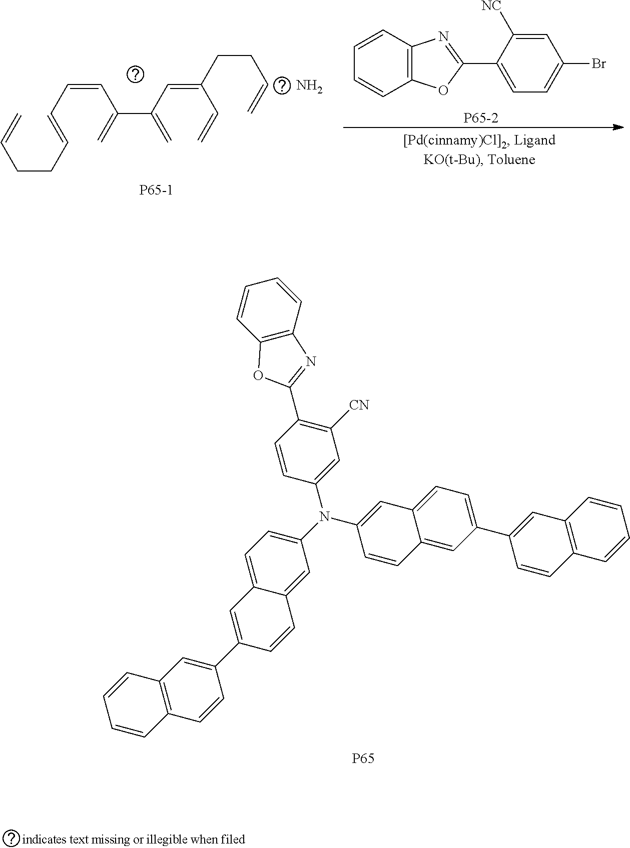

(1) P65-1 (1.0 mmol), P65-2 (0.65 mmol), KO(t-Bu) (1.5 mmol), [Pd(cinnamyl)Cl]₂ (0.04 mol) and a ligand (0.03 mol) were added to 3 mL of toluene and mixed into a solution, and the solution was put in a 50 mL flask and reacted at 80° C. for 12 h. The solution was cooled to room temperature and slowly added with a saturated aqueous solution of MgSO₄ and ethyl acetate to be extracted three times. Then, the organic layer passed through a rotary evaporator for the solvent to be removed and the crude product P65 was obtained through column chromatography.

The structure of the target product P65 was tested through matrix-assisted laser desorption/ionization time-of-flight mass spectrometry (MALDI-TOF-MS) (m/z). The structure was obtained as C₅₄H₃₃N₃O whose calculated value was 739.3 and measured value was 739.1.

Elemental analysis: theoretical value: C, 87.66; H, 4.50; N, 5.68; measured value: C, 87.66; H, 4.51; N, 5.68.

The preparation methods of the compounds of the present disclosure used in specific examples are all similar to the preceding methods and not repeated herein. Only the characterization results of these compounds are provided. The results of mass spectrometry and elemental analysis are shown in Table 1.

TABLE 1

| | Mass Spectrometry | | | |
|---|---|---|---|---|
| | Result | | | |
| | Calculated | Measured | Element Analysis Result | |
| Compound | Value | Value | Theoretical Value | Measured Value |
| P41 | 720.2 | 720.1 | C, 81.65; H, 3.92; N, 7.77; | C, 81.66; H, 3.91; N, 7.77; |
| P46 | 736.2 | 736.1 | C, 79.87; H, 3.83; N, 7.60; | C, 79.86; H, 3.83; N, 7.61; |
| P50 | 680.2 | 680.1 | C, 82.92; H, 4.15; N, 8.23; | C, 82.92; H, 4.16; N, 8.23; |
| P74 | 762.2 | 762.1 | C, 80.29; H, 3.96; N, 7.34; | C, 80.29; H, 3.95; N, 7.34; |
| P83 | 768.2 | 768.1 | C, 76.53; H, 3.67; N, 7.29; | C, 76.52; H, 3.68; N, 7.29; |

Performance Test One Characterization of Refractive Indexes of Materials

The refractive indexes of the compounds at wavelengths of 460 nm, 530 nm and 620 nm and the absorption coefficients (K) of the compounds at a wavelength of 460 nm were tested by an ellipsometer. A difference Δn₁ between a refractive index at the wavelength of 460 nm and a refractive index at the wavelength of 530 nm, a difference Δn₂ between the refractive index at the wavelength of 530 nm and a refractive index at the wavelength of 620 nm, and a difference Δn₃ between the refractive index at the wavelength of 460 nm and the refractive index at the wavelength of 620 nm were calculated.

The results of the preceding test are shown in Table 2.

TABLE 2

| Com- | K | | | | | | |
|---|---|---|---|---|---|---|---|
| pound | (n_{460 nm}) | n_{460 nm} | n_{530 nm} | n_{620 nm} | Δn₁ | Δn₂ | Δn₃ |
| P1 | 0.012 | 2.30 | 2.20 | 2.15 | 0.10 | 0.05 | 0.15 |
| P8 | 0.020 | 2.26 | 2.14 | 2.07 | 0.12 | 0.07 | 0.19 |
| P11 | 0.018 | 2.42 | 2.26 | 2.02 | 0.16 | 0.08 | 0.24 |
| P15 | 0.032 | 2.36 | 2.21 | 2.13 | 0.15 | 0.08 | 0.23 |
| P29 | 0.005 | 2.30 | 2.21 | 2.17 | 0.09 | 0.04 | 0.13 |
| P41 | 0.017 | 2.29 | 2.18 | 2.11 | 0.11 | 0.07 | 0.18 |
| P46 | 0.027 | 2.31 | 2.19 | 2.12 | 0.12 | 0.07 | 0.19 |
| P50 | 0.016 | 2.26 | 2.15 | 2.10 | 0.11 | 0.05 | 0.16 |
| P57 | 0.000 | 2.38 | 2.22 | 2.14 | 0.16 | 0.08 | 0.24 |
| P65 | 0.020 | 2.34 | 2.19 | 2.11 | 0.15 | 0.08 | 0.23 |
| P74 | 0.042 | 2.29 | 2.19 | 2.13 | 0.10 | 0.06 | 0.16 |
| P83 | 0.043 | 2.31 | 2.18 | 2.10 | 0.13 | 0.08 | 0.21 |
| C1 | 0.013 | 2.24 | 2.10 | 1.99 | 0.14 | 0.11 | 0.24 |
| C2 | 0.012 | 2.21 | 2.03 | 1.96 | 0.18 | 0.07 | 0.25 |
| C3 | 0.031 | 2.24 | 2.12 | 2.04 | 0.12 | 0.08 | 0.20 |

Comparative Compounds C1, C2 and C3 have the following structures:

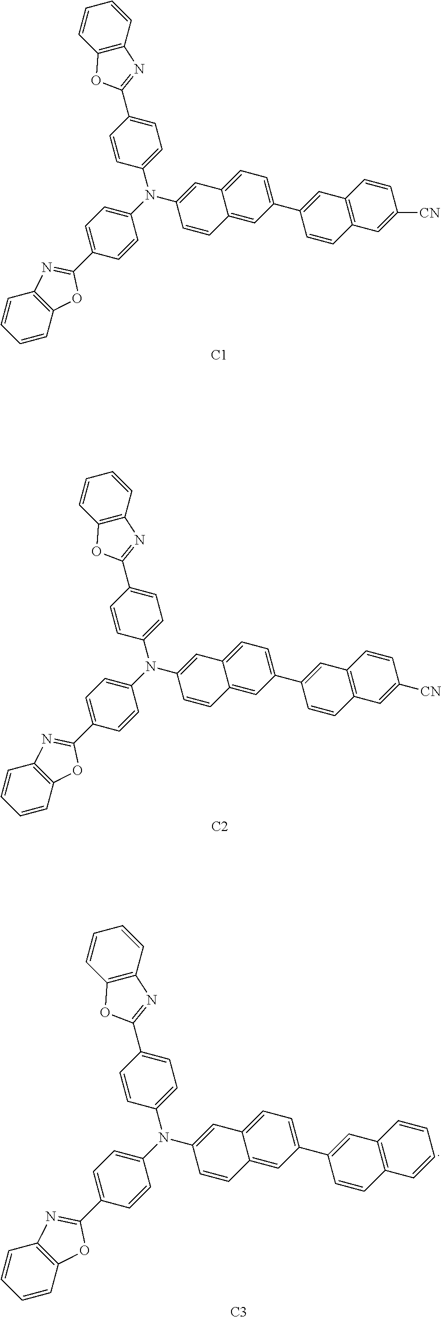 [SMILES: N#Cc1ccc2cc(-c3ccc4cc(N(c5ccc(-c6nc7ccccc7o6)cc5)c5ccc(-c6nc7ccccc7o6)cc5)ccc4c3)ccc2c1 ; "C1" ; N#Cc1ccc2cc(-c3ccc4cc(N(c5ccc(-c6nc7ccccc7o6)cc5)c5ccc(-c6nc7ccccc7o6)cc5)ccc4c3)ccc2c1 ; "C2" ; c1ccc2cc(-c3ccc4cc(N(c5ccc(-c6nc7ccccc7o6)cc5)c5ccc(-c6nc7ccccc7o6)cc5)ccc4c3)ccc2c1 ; "C3"]

It can be seen from Table 2 that the compounds of the present disclosure have higher refractive indexes in the region of visible light (400-750 nm) and satisfy that the difference between the refractive index at the wavelength of 460 nm and the refractive index at the wavelength of 530 nm is 0.10-0.17, the difference between the refractive index at the wavelength of 530 nm and the refractive index at the wavelength of 620 nm is 0.03-0.10, and the difference between the refractive index at the wavelength of 460 nm and the refractive index at the wavelength of 620 nm is 0.15-0.40. When used in an organic electroluminescent device especially as a material for a capping layer, the compounds provided by the present disclosure can effectively improve a color cast while achieving display at multiple angles. Compounds C1 and C2 differ from P1 only in that a naphthalene ring-naphthalene ring structure is replaced with a naphthalene ring-benzene ring structure and Compounds C1 and C2 cannot fully satisfy the above differences between refractive indexes and are difficult to achieve display at multiple angles; Compound C3 differs from P1 only in that no cyano groups are substituted and Compound C3 has a reduced refractive index and a relatively large value of K, absorbs more blue light, and achieves lower device efficiency than P1 when used as the material for the capping layer.

For a better understanding of the present disclosure, application examples of the compounds of the present disclosure are listed below. It is to be understood by those skilled in the art that the examples described herein are used for a better understanding of the present disclosure and not to be construed as specific limitations to the present disclosure.

### Application Example 1

This application example provides an organic electroluminescent device which has a structure shown in FIG. 1 and is prepared through specific steps described below.

(1) A glass substrate with an indium tin oxide (ITO) anode layer 2 (with a thickness of 15 nm) was cut into a size of 50 mm×50 mm×0.7 mm, sonicated in isopropyl alcohol and deionized water for 30 min separately, and cleaned under ozone for 10 min. The obtained substrate 1 was installed onto a vacuum deposition device.

(2) A material for a hole injection layer, Compound 2, and a p-doping material, Compound 1, were co-deposited at a doping ratio of 3% (mass ratio) by means of vacuum evaporation on the ITO anode layer 2 as a hole injection layer 3 with a thickness of 5 nm.

(3) A material for a hole transport layer, Compound 2, was deposited by means of vacuum evaporation on the hole injection layer 3 as a first hole transport layer 4 with a thickness of 100 nm.

(4) A hole transport material, Compound 3, was deposited by means of vacuum evaporation on the first hole transport layer 4 as a second hole transport layer 5 with a thickness of 5 nm.

(5) A light-emitting layer 6 with a thickness of 30 nm was deposited by means of vacuum evaporation on the second hole transport layer 5, where Compound 4 was doped as a host material with Compound 5 as a doping material at a ratio of 3% (mass ratio).

(6) An electron transport material, Compound 6, was deposited by means of vacuum evaporation on the light-emitting layer 6 as a first electron transport layer 7 with a thickness of 30 nm.

(7) An electron transport material, Compound 7, and an n-doping material,

Compound 8, were co-deposited at a doping mass ratio of 1:1 by means of vacuum evaporation on the first electron transport layer 7 as a second electron transport layer 8 with a thickness of 5 nm.

(8) A magnesium-silver electrode was deposited at a ratio (9:1) of Mg:Ag by means of vacuum evaporation on the second electron transport layer 8 as a cathode 9 with a thickness of 10 nm.

(9) Compound P1 of the present disclosure was deposited by means of vacuum evaporation on the cathode 9 as a capping layer 10 with a thickness of 100 nm.

The compounds used in the preceding steps have the following structures:

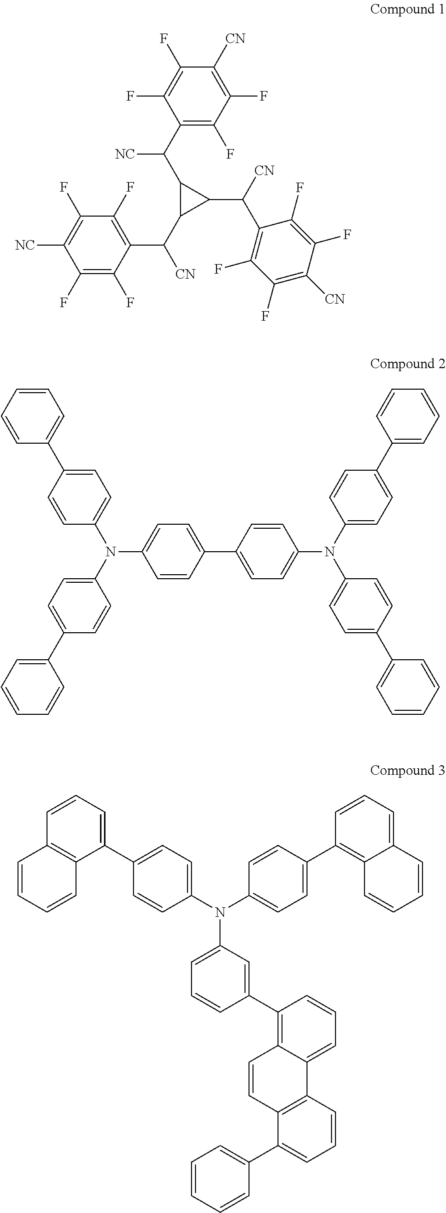 [SMILES: N#Cc1c(F)c(F)c(C(C#N)C2C(C(C#N)c3c(F)c(F)c(C#N)c(F)c3F)C2C(C#N)c2c(F)c(F)c(C#N)c(F)c2F)c(F)c1F ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2cccc3c2ccc2c(-c4cccc(N(c5ccc(-c6cccc7ccccc67)cc5)c5ccc(-c6cccc7ccccc67)cc5)c4)cccc23)cc1]

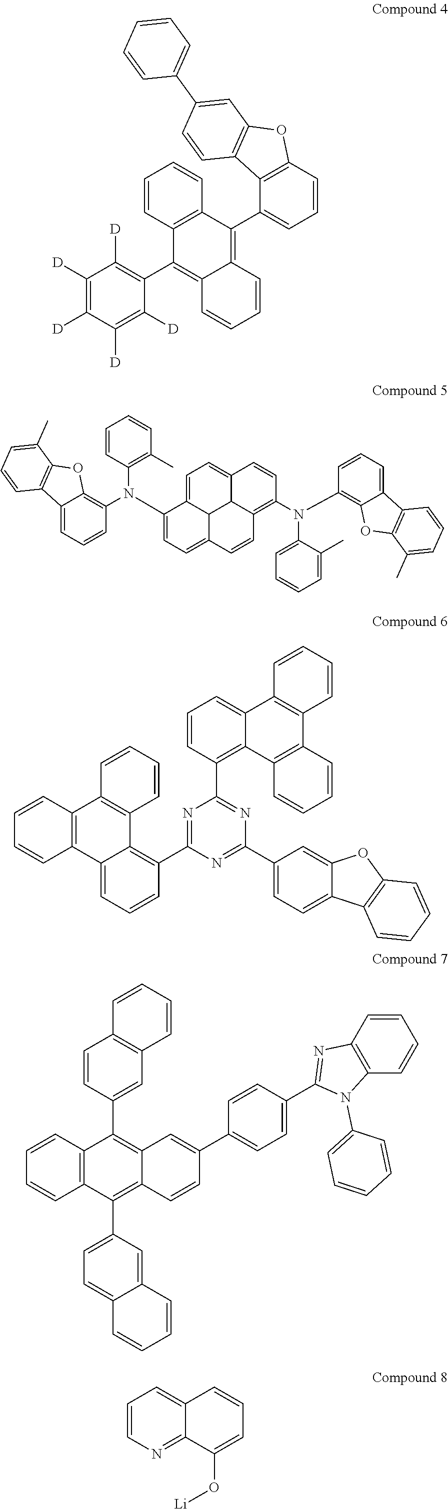

Application Examples 2-12 and Comparative Application Examples 1-3 differ from Application Example 1 only in that Compound P1 in step (9) was replaced with Compounds P8, P11, P15, P29, P41, P46, P50, P57, P65, P74, P83, C1, C2 and C3 respectively for preparing the capping layer. All the other preparation steps are the same. For details, see Table 3.

Performance Test Two Characterization of Device Performance

A performance test was performed on organic electroluminescent devices provided in Application Examples 1-12 and Comparative Application Examples 1-3 as follows.

Currents were measured with Keithley 2365A digital nanovoltmeter at different voltages for the organic electroluminescent devices and then divided by a light-emitting area so that the current densities of the organic optoelectronic devices at different voltages were obtained. The brightness and radiation energy flux density of each of the organic electroluminescent devices manufactured according to application examples and comparative application examples at different voltages were tested with Konicaminolta CS-2000 spectroradiometer. According to the current densities and brightness of the organic electroluminescent devices at different voltages, an operating voltage Vₒₙ (V), current efficiency CE (cd/A), external quantum efficiency EQE₍ₘₐₓ₎, a color cast JNCD (30/45/60° C.) and a lifetime LT95 (which is obtained by measuring time taken for the organic electroluminescent device to reach 95% of initial brightness (under a condition of 50 mA/cm²)) at the same current density (10 mA/cm²) were obtained. The results are shown in Table 3.

TABLE 3

| | | | | | | Life- |
|---|---|---|---|---|---|---|
| | | | | | | time |
| | Com- | Vₒₙ | CE_{(10 mA/cm}²₎ | | EQE₍ₘₐₓ₎ | LT95 |
| No. | pound | (V) | (cd A⁻¹) | JNCD | (%) | (h) |
| Application | P1 | 3.41 | 8.22 | 4/1/1 | 18.1 | 73 |
| Example 1 | | | | | | |
| Application | P8 | 3.42 | 8.19 | 4/4/2 | 17.6 | 70 |
| Example 2 | | | | | | |
| Application | P11 | 3.43 | 8.33 | 5/3/1 | 18.9 | 70 |
| Example 3 | | | | | | |
| Application | P15 | 3.41 | 8.16 | 5/2/1 | 17.7 | 89 |
| Example 4 | | | | | | |
| Application | P29 | 3.40 | 8.21 | 4/3/2 | 18.1 | 72 |
| Example 5 | | | | | | |
| Application | P41 | 3.44 | 8.20 | 3/2/1 | 18.8 | 70 |
| Example 6 | | | | | | |
| Application | P46 | 3.42 | 8.18 | 4/3/3 | 17.1 | 71 |
| Example 7 | | | | | | |
| Application | P50 | 3.41 | 8.17 | 4/3/1 | 17.3 | 71 |
| Example 8 | | | | | | |
| Application | P57 | 3.40 | 8.19 | 3/3/1 | 18.0 | 70 |
| Example 9 | | | | | | |
| Application | P65 | 3.39 | 8.12 | 4/3/2 | 18.6 | 69 |
| Example 10 | | | | | | |
| Application | P74 | 3.44 | 8.19 | 4/3/1 | 17.4 | 69 |
| Example 11 | | | | | | |
| Application | P83 | 3.41 | 8.18 | 4/4/2 | 17.6 | 72 |
| Example 12 | | | | | | |
| Comparative | C1 | 3.42 | 7.85 | 4/4/4 | 18.0 | 70 |
| Application | | | | | | |
| Example 1 | | | | | | |
| Comparative | C2 | 3.41 | 7.83 | 5/3/2 | 17.8 | 71 |
| Application | | | | | | |
| Example 2 | | | | | | |
| Comparative | C3 | 3.42 | 7.86 | 4/3/1 | 18.1 | 71 |
| Application | | | | | | |
| Example 3 | | | | | | |

It can be seen from Table 3 that when used as the material in the capping layer of the organic electroluminescent device, the compound of the present disclosure can effectively reduce the color cast of the device and improve current efficiency and external quantum efficiency.

Compounds C1 and C2 differ from P1 only in that the naphthalene ring-naphthalene ring structure is replaced with the naphthalene ring-benzene ring structure so that devices using C1 and C2 have lower current efficiency and external quantum efficiency than the device using P1 and have relatively serious color cast. Compound C3 differs from P1 only in that no cyano groups are substituted so that the device using C3 has lower current efficiency than the device using P1. This proves that the introduction of a fused ring-fused ring structure and a cyano group into the compound can further improve device performance.

Compound P15 used in Application Example 4 has a deuterated structure and can effectively improve the lifetime of the device compared with a compound with a non-deuterated structure. The device efficiency is affected by the refractive index n and the extinction coefficient K of the material. P15 corresponds to a large n value and a relatively large K value. Therefore, the device efficiency of Application Example 4 is similar to those of the other application examples.

### Application Example 13

This application example provides an organic electroluminescent device which has a structure shown in FIG. 2 and is prepared through specific steps described below.

(1) A glass substrate with an indium tin oxide (ITO) anode layer 2 (with a thickness of 15 nm) was cut into a size of 50 mm×50 mm×0.7 mm was cut, sonicated in isopropyl alcohol and deionized water for 30 min separately, and cleaned under ozone for 10 min. The obtained substrate 1 was installed onto a vacuum deposition device.

(2) A material for a hole injection layer, Compound 2, and a p-doping material, Compound 1, were co-deposited at a doping ratio of 3% (mass ratio) by means of vacuum evaporation on the ITO anode layer 2 as a hole injection layer 3 with a thickness of 5 nm.

(3) A material for a hole transport layer, Compound 2, was deposited by means of vacuum evaporation on the hole injection layer 3 as a first hole transport layer 4 with a thickness of 100 nm.

(4) A hole transport material, Compound 3, was deposited by means of vacuum evaporation on the first hole transport layer 4 as a second hole transport layer 5 with a thickness of 5 nm.

(5) A light-emitting layer 6 with a thickness of 30 nm was deposited by means of vacuum evaporation on the second hole transport layer 5, where Compound 4 was doped as a host material with Compound 5 as a doping material at a ratio of 3% (mass ratio).

(6) An electron transport material, Compound 6, was deposited by means of vacuum evaporation on the light-emitting layer 6 as a first electron transport layer 7 with a thickness of 30 nm.

(7) An electron transport material, Compound 7, and an n-doping material, Compound 8, were co-deposited at a doping mass ratio of 1:1 by means of vacuum evaporation on the first electron transport layer 7 as a second electron transport layer 8 with a thickness of 5 nm.

(8) A magnesium-silver electrode was deposited at a ratio (9:1) of Mg:Ag by means of vacuum evaporation on the second electron transport layer 8 as a cathode 9 with a thickness of 10 nm.

(9) Compound P1 of the present disclosure was deposited by means of vacuum evaporation on the cathode 9 as a first capping layer 10 with a thickness of 100 nm.

(10) A small organic molecule D1 with a low refractive index was deposited by means of vacuum evaporation on the first capping layer 10 as a second capping layer 11 with a thickness of 20 nm.

The small organic molecules with low refractive indexes have the following structures:

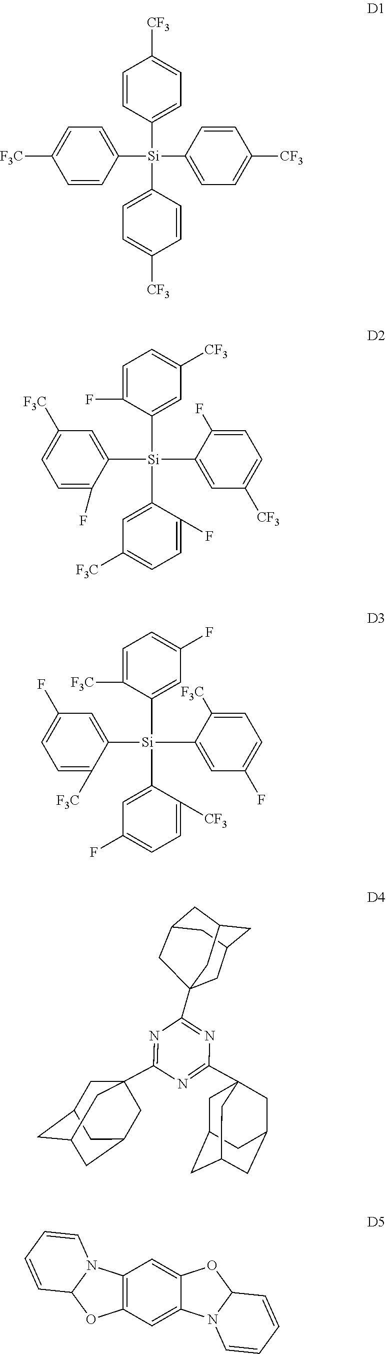 [SMILES: FC(F)(F)c1ccc([Si](c2ccc(C(F)(F)F)cc2)(c2ccc(C(F)(F)F)cc2)c2ccc(C(F)(F)F)cc2)cc1 ; Fc1ccc(C(F)(F)F)cc1[Si](c1cc(C(F)(F)F)ccc1F)(c1cc(C(F)(F)F)ccc1F)c1cc(C(F)(F)F)ccc1F ; Fc1ccc(C(F)(F)F)c([Si](c2cc(F)ccc2C(F)(F)F)(c2cc(F)ccc2C(F)(F)F)c2cc(F)ccc2C(F)(F)F)c1 ; C1C2CC3CC1CC(c1nc(C45CC6CC(CC(C6)C4)C5)nc(C45CC6CC(CC(C6)C4)C5)n1)(C2)C3 ; C1=CC2Oc3cc4c(cc3N2C=C1)OC1C=CC=CN41]

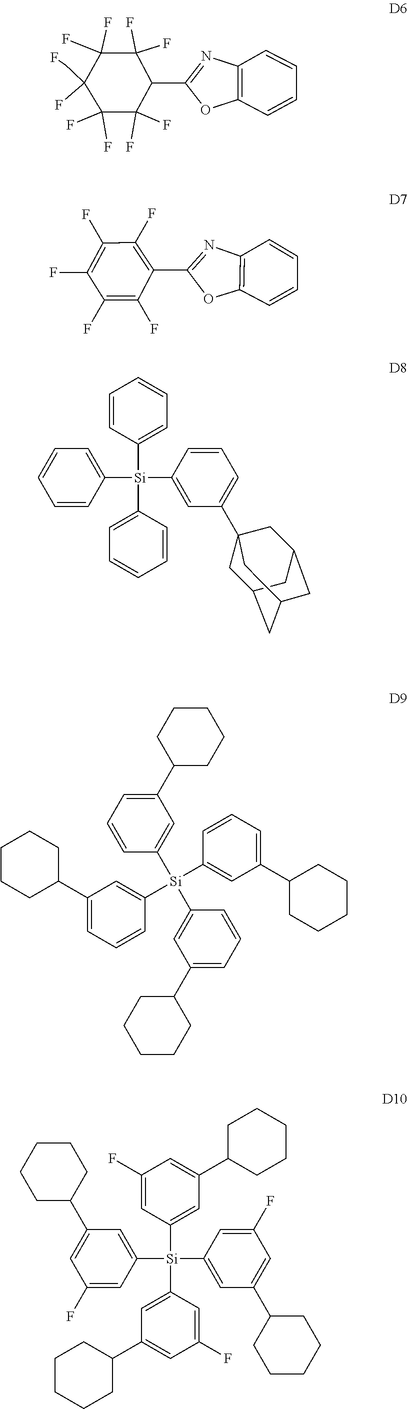 [SMILES: FCC1(F)C(F)(F)C(F)(F)C(c2nc3ccccc3o2)C(F)(F)C1(F)F ; Fc1c(F)c(F)c(-c2nc3ccccc3o2)c(F)c1F ; c1ccc([Si](c2ccccc2)(c2ccccc2)c2cccc(C34CC5CC(CC(C5)C3)C4)c2)cc1 ; c1cc(C2CCCCC2)cc([Si](c2cccc(C3CCCCC3)c2)(c2cccc(C3CCCCC3)c2)c2cccc(C3CCCCC3)c2)c1 ; Fc1cc(C2CCCCC2)cc([Si](c2cc(F)cc(C3CCCCC3)c2)(c2cc(F)cc(C3CCCCC3)c2)c2cc(F)cc(C3CCCCC3)c2)c1]

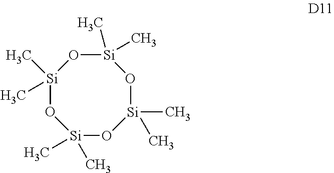

Application Examples 14-23 differ from Application Example 13 only in that the small organic molecule D1 in step (10) was replaced with D2, D3, D4, D5, D6, D7, D8, D9, D10 and D11 respectively for preparing the second capping layer. All the other preparation steps are the same. Application Examples 24-26 and Comparative Application Examples 4-6 differ from Application Example 13 only in that Compound P1 in step (9) was replaced with P8, P11, P15, C1, C2 and C3 respectively for preparing the first capping layer. For details, see Table 4.

The performance test was performed on organic electroluminescent devices provided in Application Examples 13-26 and Comparative Application Examples 4-6 by the same test method described above. The results are shown in Table 4.

TABLE 4

| | Material for | Material for | | |
|---|---|---|---|---|
| | First | Second | CE_{(10 mA/cm}²₎ | EQE₍ₘₐₓ₎ |
| No. | Capping Layer | Capping Layer | (cd A⁻¹) | (%) |
| Application | P1 | D1 | 8.30 | 19.9 |
| Example 13 | | | | |
| Application | P1 | D2 | 8.32 | 20.1 |
| Example 14 | | | | |
| Application | P1 | D3 | 8.32 | 19.8 |
| Example 15 | | | | |
| Application | P1 | D4 | 8.29 | 19.4 |
| Example 16 | | | | |
| Application | P1 | D5 | 8.28 | 19.5 |
| Example 17 | | | | |
| Application | P1 | D6 | 8.23 | 19.9 |
| Example 18 | | | | |
| Application | P1 | D7 | 8.20 | 19.7 |
| Example 19 | | | | |
| Application | P1 | D8 | 8.26 | 19.6 |
| Example 20 | | | | |
| Application | P1 | D9 | 8.27 | 19.6 |
| Example 21 | | | | |
| Application | P1 | D10 | 8.29 | 19.4 |
| Example 22 | | | | |
| Application | P1 | D11 | 8.25 | 19.3 |
| Example 23 | | | | |
| Application | P8 | D1 | 8.33 | 20.0 |
| Example 24 | | | | |
| Application | P11 | D1 | 8.36 | 20.1 |
| Example 25 | | | | |
| Application | P15 | D1 | 8.35 | 20.1 |
| Example 26 | | | | |
| Comparative | C1 | D1 | 7.92 | 18.4 |
| Application | | | | |
| Example 4 | | | | |
| Comparative | C2 | D1 | 7.89 | 18.3 |
| Application | | | | |
| Example 5 | | | | |
| Comparative | C3 | D1 | 7.92 | 18.5 |
| Application | | | | |
| Example 6 | | | | |

It can be seen from Table 4 that compared with the use of Compound C1, C2 or C3 with the material used in the second capping layer and containing the small organic molecule with a low refractive index, the use of the compound provided by the present disclosure as the material in the first capping layer with the material used in the second capping layer and containing the small organic molecule with a low refractive index is more conducive to improving device efficiency, especially in terms of increasing external quantum efficiency.

The applicant has stated that although the detailed method of the present disclosure is described through the examples described above, the present disclosure is not limited to the detailed method described above, which means that the implementation of the present disclosure does not necessarily depend on the detailed method described above. It should be apparent to those skilled in the art that any improvements made to the present disclosure, equivalent substitutions of various raw materials of the product, the addition of adjuvant ingredients, and the selection of specific manners, etc. in the present disclosure all fall within the protection scope and the scope of disclosure of the present disclosure.

What is claimed is:

1. A compound having a structure which is formed by substituting at any substitutable position of a structure represented by Formula I with at least one cyano group: 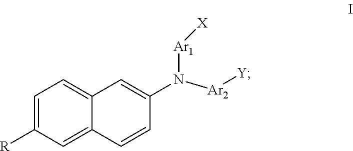
wherein
in Formula I, R is selected from any one of substituted or unsubstituted fused ring C10-C60 aryl or substituted or unsubstituted fused ring C6-C60 heteroaryl;
in Formula I, Ar₁ and Ar₂ are each independently selected from any one of a single bond, substituted or unsubstituted C6-C60 arylene, substituted or unsubstituted C3-C60 heteroarylene, substituted or unsubstituted fused ring C10-C60 arylene or substituted or unsubstituted fused ring C6-C60 heteroarylene; and
in Formula I, X and Y are each independently selected from substituted or unsubstituted C6-C60 aryl or substituted or unsubstituted C3-C60 heteroaryl, and at least one of X or Y is selected from substituted or unsubstituted C3-C60 electron withdrawing heteroaryl;
wherein substituents in R, Ar₁, Ar₂, X and Y are each independently selected from any one or a combination of at least two of protium, deuterium, tritium, halogen, C1-C10 alkyl, C1-C10 haloalkyl, C1-C10 alkoxy, C6-C60 aryl and C3-C60 heteroaryl.

2. The compound according to claim 1, wherein the number of the cyano group(s) in the compound is 1 to 10.

3. The compound according to claim 1, wherein at least one cyano group substituent is on the group R.

4. The compound according to claim 1, wherein at least one cyano group substituent is on Ar₁, Ar₂, X or Y.

5. The compound according to claim 1, wherein the compound has a structure represented by Formula (1), Formula (2), Formula (3) or Formula (4): 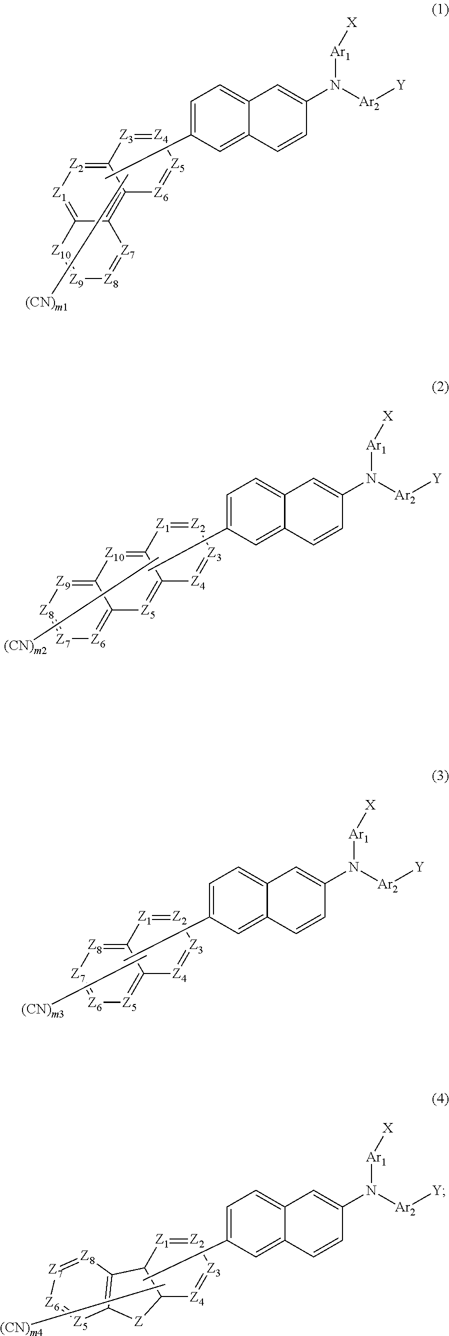
wherein
m1 and m2 are each independently selected from an integer from 1 to 9, and m3 and m4 are each independently selected from an integer from 1 to 7;
Z is selected from O or S;
Z₁ to Z₁₀ are each independently selected from a N atom, CH or CR₁, wherein R₁ is selected from any one or a combination of at least two of protium, deuterium, tritium, halogen, C1-C10 alkyl, C1-C10 haloalkyl, C1-C10 alkoxy, C6-C60 aryl or C3-C60 heteroaryl; and
Ar₁, Ar₂, X and Y each have the same range as defined in Formula I.

6. The compound according to claim 5, wherein the compound has any one of the following structures: 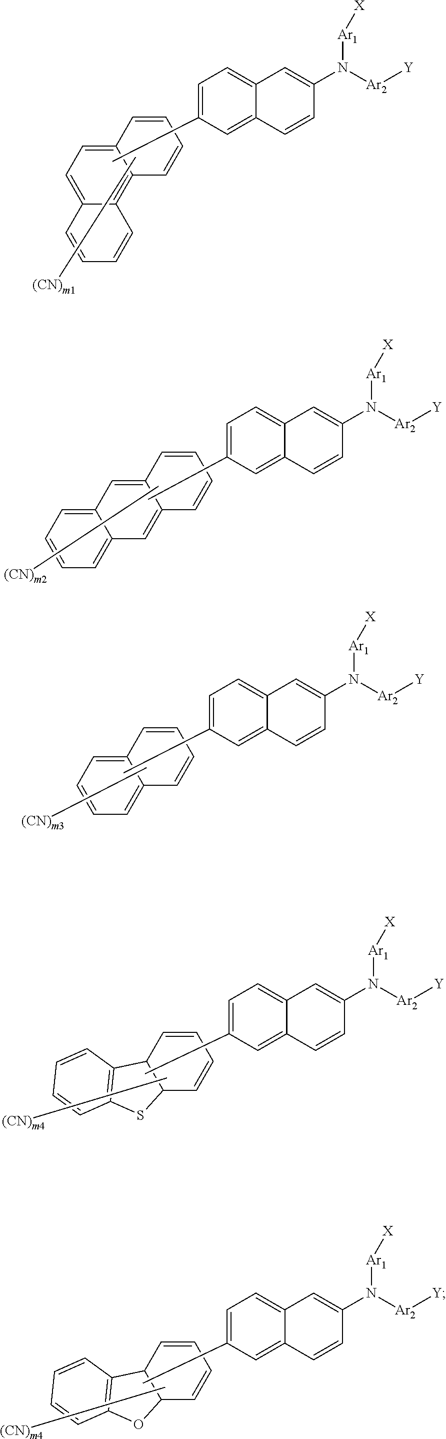
wherein
m1 and m2 are each independently selected from an integer from 1 to 9, and m3 and m4 are each independently selected from an integer from 1 to 7; and
Ar₁, Ar₂, X and Y each have the same range as defined in Formula I.

7. The compound according to claim 6, wherein the compound has any one of the following structures: 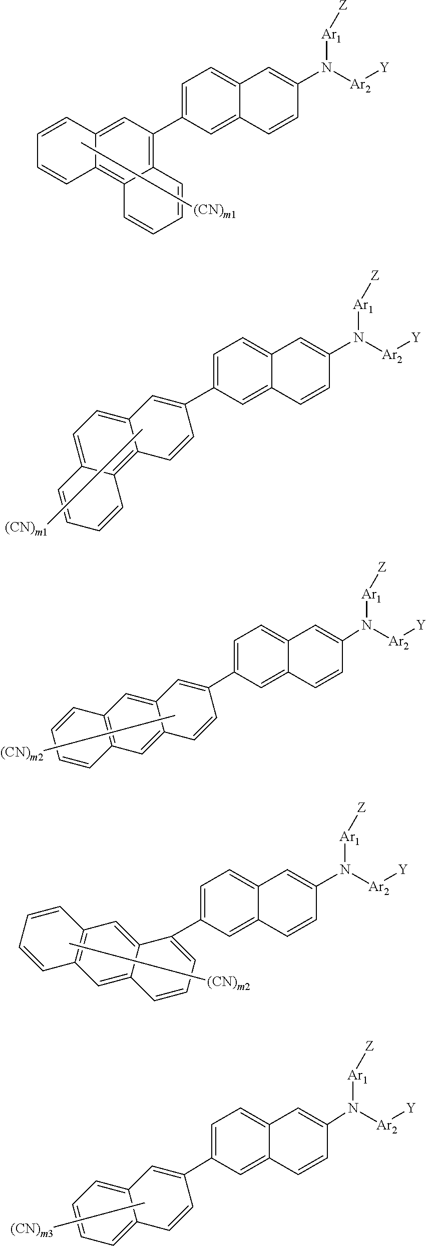 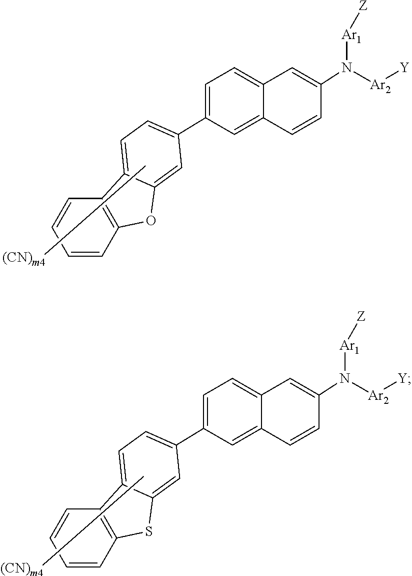
wherein
m1 and m2 are each independently selected from an integer from 1 to 9, and m3 and m4 are each independently selected from an integer from 1 to 7; and
Ar₁, Ar₂, X and Y each have the same range as defined in Formula I.

8. The compound according to claim 1, wherein R is selected from any one of groups represented by Formula (5), Formula (6) or Formula (7): 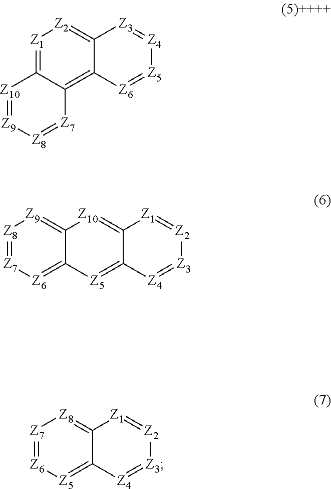
wherein Z₁ to Z₁₀ are each independently selected from a N atom, CH or CR₁, wherein R₁ is selected from any one or a combination of at least two of protium, deuterium, tritium, halogen, C1-C10 alkyl, C1-C10 haloalkyl, C1-C10 alkoxy, C6-C60 aryl or C3-C60 heteroaryl.

9. The compound according to claim 1, wherein R is selected from any one of the following groups: 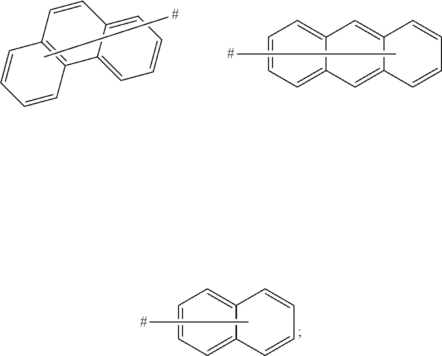
wherein # represents a linkage site of the group.

10. The compound according to claim 1, wherein R is selected from any one of the following groups: 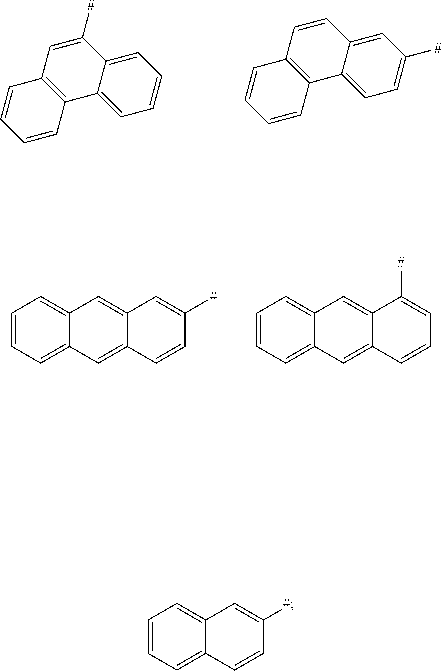
wherein # represents a linkage site of the group.

11. The compound according to claim 1, wherein X and Y are each independently selected from substituted or unsubstituted C3-C60 electron withdrawing heteroaryl.

12. The compound according to claim 1, wherein X and Y are each independently selected from any one of the following substituted or unsubstituted groups: 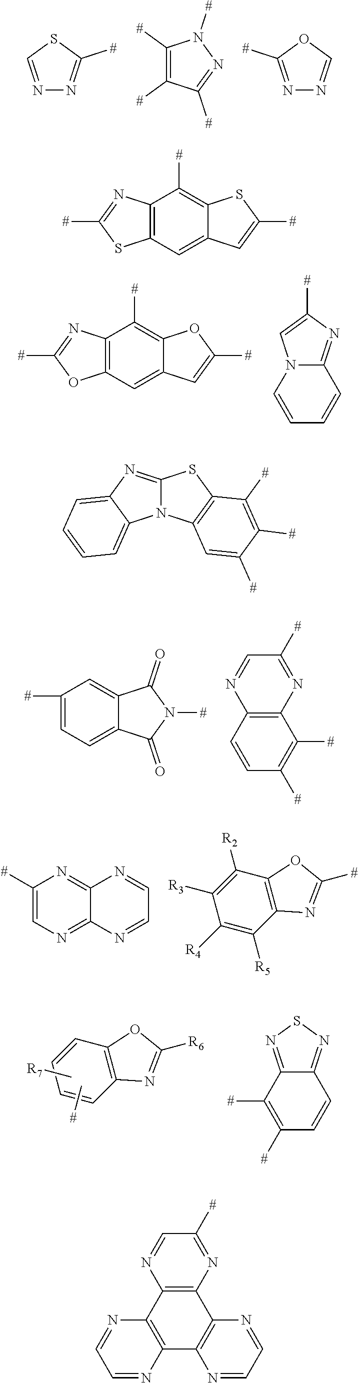 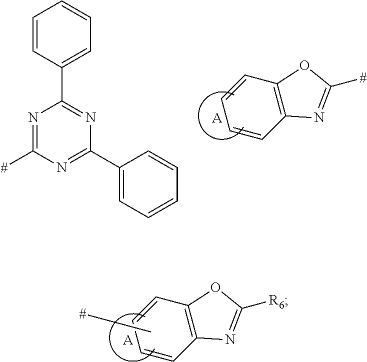
wherein
# represents a linkage site of the group;
Q is selected from an O atom, a S atom or NR₈;
R₂ to R₈ are each independently selected from any one of hydrogen, protium, deuterium, tritium, halogen, C1-C10 alkyl, C1-C10 alkoxy, C6-C60 aryl or C3-C60 heteroaryl; and
the ring A is fused at any available position of the benzene ring and the ring A is selected from substituted or unsubstituted C6-C30 aromatic rings or substituted or unsubstituted C3-C30 heteroaromatic rings.

13. The compound according to claim 12, wherein X and Y are each independently selected from any one of the following groups: 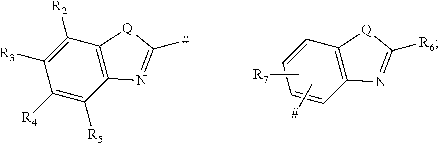
wherein
# represents a linkage site of the group; and
Q is selected from an O atom, a S atom or NR₈; and
R₂ to R₇ are each independently selected from any one of hydrogen, protium, deuterium, tritium, halogen, C1-C10 alkyl, C1-C10 alkoxy, C6-C60 aryl or C3-C60 heteroaryl.

14. The compound according to claim 13, wherein X and Y are each independently selected from any one of the following groups: 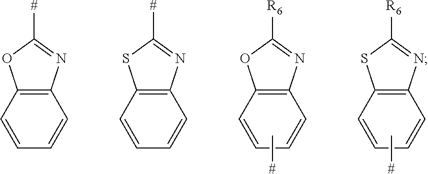
wherein
# represents a linkage site of the group; and
R₆ is independently selected from any one of hydrogen, protium, deuterium, tritium, halogen, C1-C10 alkyl, C1-C10 alkoxy, C6-C60 aryl or C3-C60 heteroaryl.

15. The compound according to claim 1, wherein Ar₁ and Ar₂ are each independently selected from any one of phenylene, biphenylene, terphenylene, naphthylene, anthrylene, phenanthrenylene, pyridinylene, pyrimidinylene, triazinylene, furylene, pyrrolidene, thienylene, quinolylene, isoquinolylene, benzofurylene or benzothienylene.

16. The compound according to claim 1, wherein the compound has any one of the following structures represented by P1 to P102:

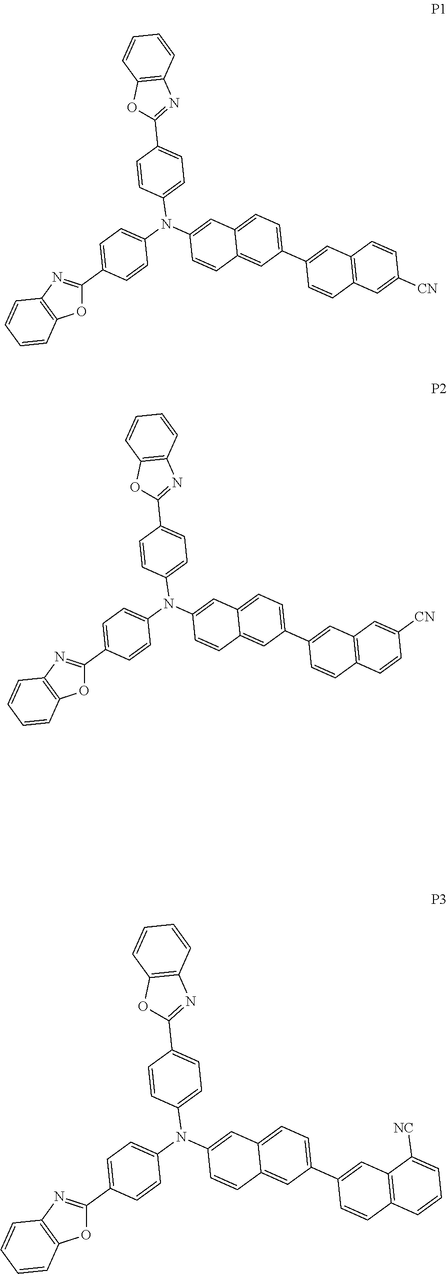 [SMILES: N#Cc1ccc2cc(-c3ccc4cc(N(c5ccc(-c6nc7ccccc7o6)cc5)c5ccc(-c6nc7ccccc7o6)cc5)ccc4c3)ccc2c1 ; N#Cc1ccc2ccc(-c3ccc4cc(N(c5ccc(-c6nc7ccccc7o6)cc5)c5ccc(-c6nc7ccccc7o6)cc5)ccc4c3)cc2c1 ; N#Cc1cccc2ccc(-c3ccc4cc(N(c5ccc(-c6nc7ccccc7o6)cc5)c5ccc(-c6nc7ccccc7o6)cc5)ccc4c3)cc12]

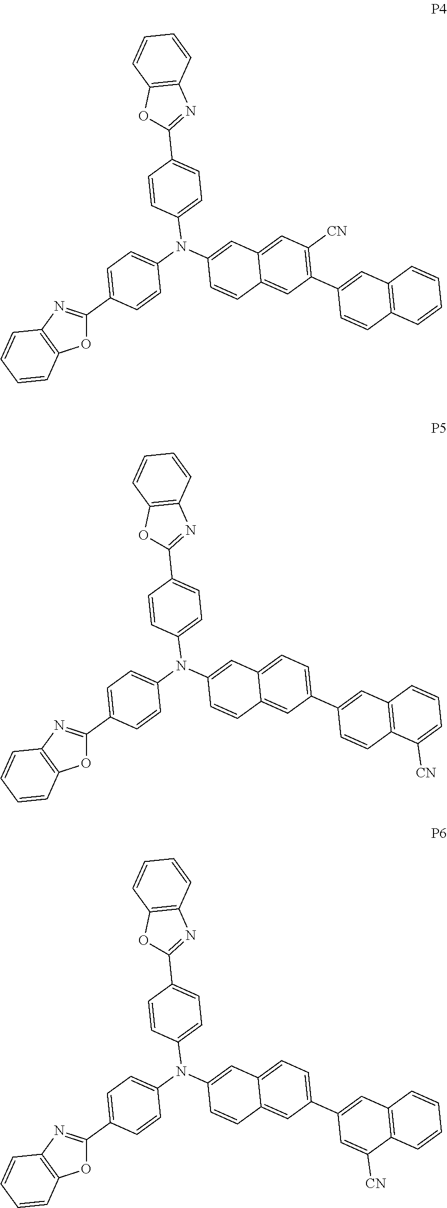 [SMILES: N#Cc1cc2cc(N(c3ccc(-c4nc5ccccc5o4)cc3)c3ccc(-c4nc5ccccc5o4)cc3)ccc2cc1-c1ccc2ccccc2c1 ; N#Cc1cccc2cc(-c3ccc4cc(N(c5ccc(-c6nc7ccccc7o6)cc5)c5ccc(-c6nc7ccccc7o6)cc5)ccc4c3)ccc12 ; N#Cc1cc(-c2ccc3cc(N(c4ccc(-c5nc6ccccc6o5)cc4)c4ccc(-c5nc6ccccc6o5)cc4)ccc3c2)cc2ccccc12]

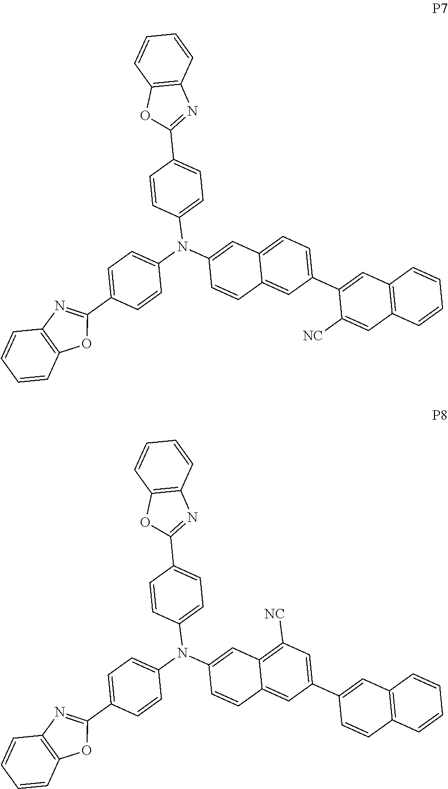 [SMILES: N#Cc1cc2ccccc2cc1-c1ccc2cc(N(c3ccc(-c4nc5ccccc5o4)cc3)c3ccc(-c4nc5ccccc5o4)cc3)ccc2c1 ; N#Cc1cc(-c2ccc3ccccc3c2)cc2ccc(N(c3ccc(-c4nc5ccccc5o4)cc3)c3ccc(-c4nc5ccccc5o4)cc3)cc12]

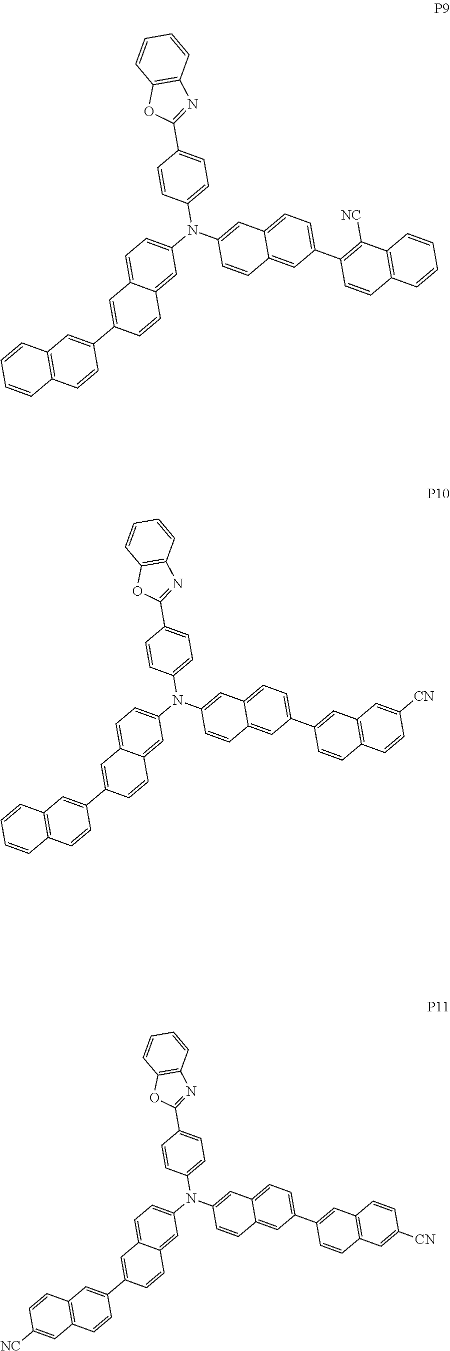 [SMILES: N#Cc1c(-c2ccc3cc(N(c4ccc(-c5nc6ccccc6o5)cc4)c4ccc5cc(-c6ccc7ccccc7c6)ccc5c4)ccc3c2)ccc2ccccc12 ; N#Cc1ccc2ccc(-c3ccc4cc(N(c5ccc(-c6nc7ccccc7o6)cc5)c5ccc6cc(-c7ccc8ccccc8c7)ccc6c5)ccc4c3)cc2c1 ; N#Cc1ccc2cc(-c3ccc4cc(N(c5ccc(-c6nc7ccccc7o6)cc5)c5ccc6cc(-c7ccc8cc(C#N)ccc8c7)ccc6c5)ccc4c3)ccc2c1]

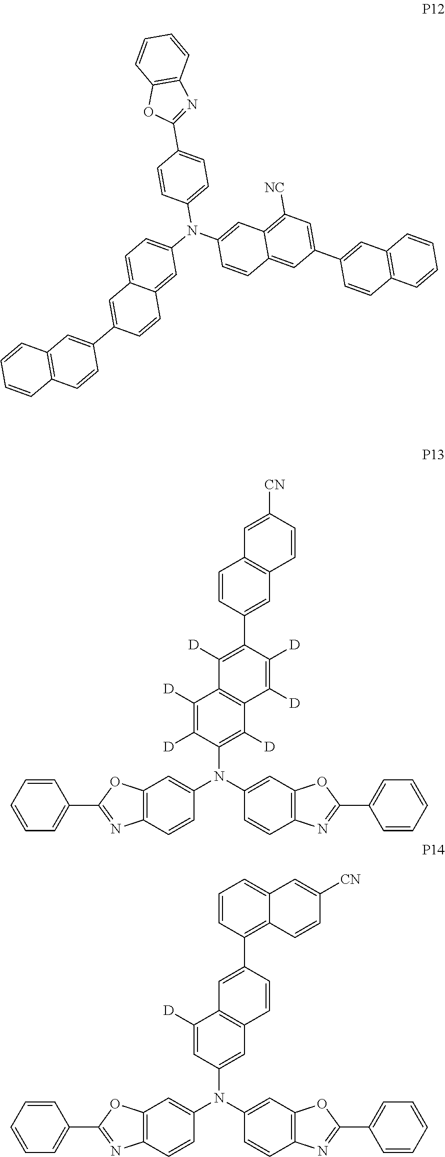 [SMILES: N#Cc1cc(-c2ccc3ccccc3c2)cc2ccc(N(c3ccc(-c4nc5ccccc5o4)cc3)c3ccc4cc(-c5ccc6ccccc6c5)ccc4c3)cc12 ; [2H]c1c(N(c2ccc3nc(-c4ccccc4)oc3c2)c2ccc3nc(-c4ccccc4)oc3c2)c([2H])c2c([2H])c([2H])c(-c3ccc4cc(C#N)ccc4c3)c([2H])c2c1[2H] ; [2H]c1cc(N(c2ccc3nc(-c4ccccc4)oc3c2)c2ccc3nc(-c4ccccc4)oc3c2)cc2ccc(-c3cccc4cc(C#N)ccc34)cc12]

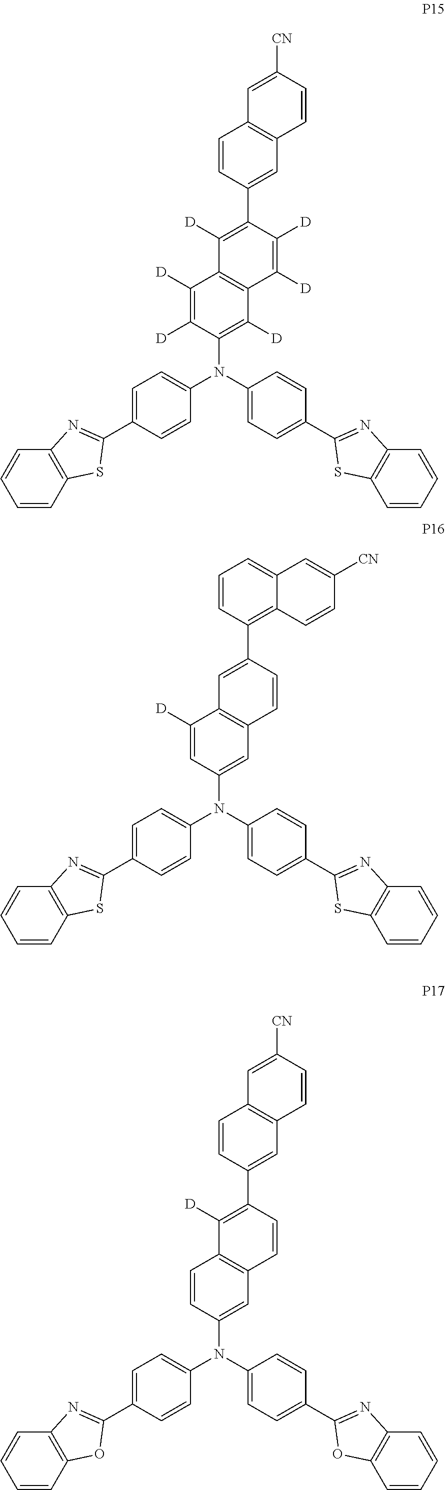 [SMILES: [2H]c1c(N(c2ccc(-c3nc4ccccc4s3)cc2)c2ccc(-c3nc4ccccc4s3)cc2)c([2H])c2c([2H])c([2H])c(-c3ccc4cc(C#N)ccc4c3)c([2H])c2c1[2H] ; [2H]c1cc(N(c2ccc(-c3nc4ccccc4s3)cc2)c2ccc(-c3nc4ccccc4s3)cc2)cc2ccc(-c3cccc4cc(C#N)ccc34)cc12 ; [2H]c1c(-c2ccc3cc(C#N)ccc3c2)ccc2cc(N(c3ccc(-c4nc5ccccc5o4)cc3)c3ccc(-c4nc5ccccc5o4)cc3)ccc12]

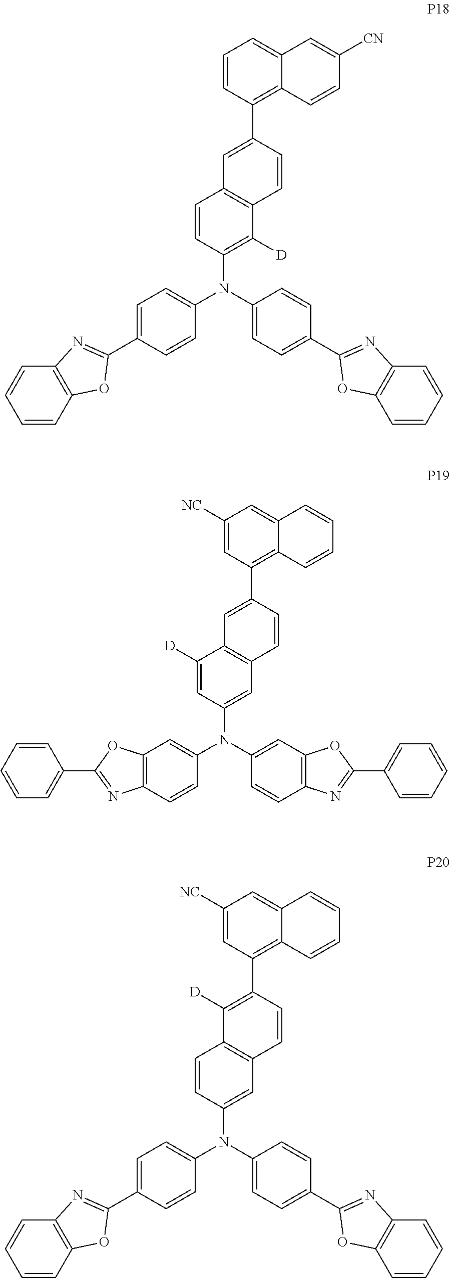 [SMILES: [2H]c1c(N(c2ccc(-c3nc4ccccc4o3)cc2)c2ccc(-c3nc4ccccc4o3)cc2)ccc2cc(-c3cccc4cc(C#N)ccc34)ccc12 ; [2H]c1cc(N(c2ccc3nc(-c4ccccc4)oc3c2)c2ccc3nc(-c4ccccc4)oc3c2)cc2ccc(-c3cc(C#N)cc4ccccc34)cc12 ; [2H]c1c(-c2cc(C#N)cc3ccccc23)ccc2cc(N(c3ccc(-c4nc5ccccc5o4)cc3)c3ccc(-c4nc5ccccc5o4)cc3)ccc12]

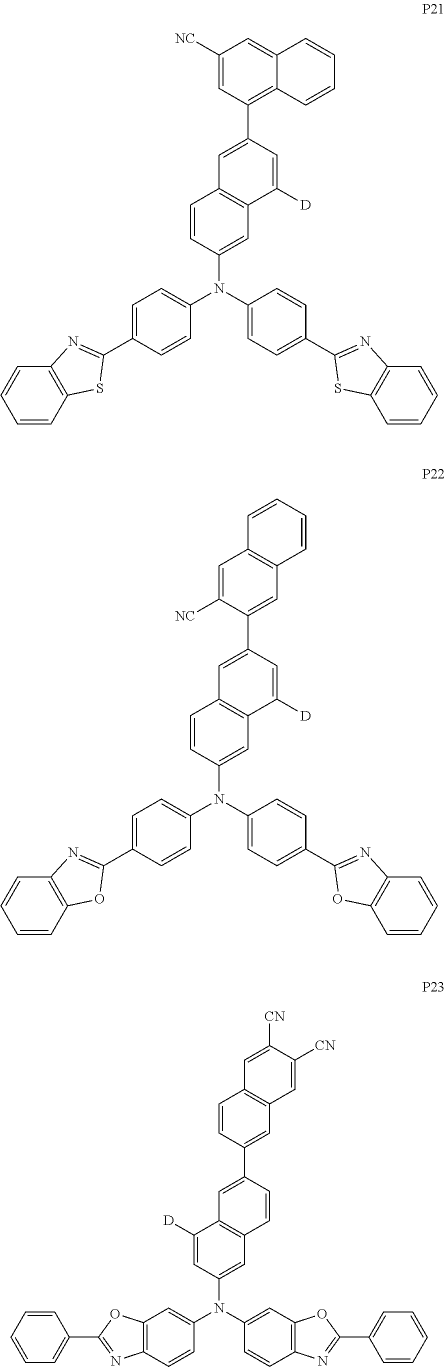 [SMILES: [2H]c1cc(-c2cc(C#N)cc3ccccc23)cc2ccc(N(c3ccc(-c4nc5ccccc5s4)cc3)c3ccc(-c4nc5ccccc5s4)cc3)cc12 ; [2H]c1cc(-c2cc3ccccc3cc2C#N)cc2ccc(N(c3ccc(-c4nc5ccccc5o4)cc3)c3ccc(-c4nc5ccccc5o4)cc3)cc12 ; [2H]c1cc(N(c2ccc3nc(-c4ccccc4)oc3c2)c2ccc3nc(-c4ccccc4)oc3c2)cc2ccc(-c3ccc4cc(C#N)c(C#N)cc4c3)cc12]

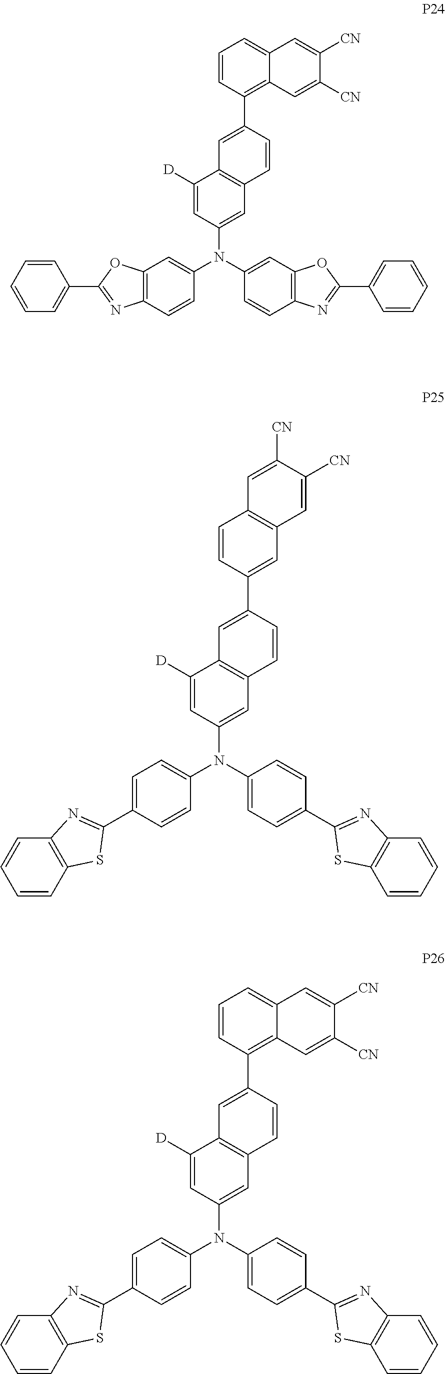 [SMILES: [2H]c1cc(N(c2ccc3nc(-c4ccccc4)oc3c2)c2ccc3nc(-c4ccccc4)oc3c2)cc2ccc(-c3cccc4cc(C#N)c(C#N)cc34)cc12 ; [2H]c1cc(N(c2ccc(-c3nc4ccccc4s3)cc2)c2ccc(-c3nc4ccccc4s3)cc2)cc2ccc(-c3ccc4cc(C#N)c(C#N)cc4c3)cc12 ; [2H]c1cc(N(c2ccc(-c3nc4ccccc4s3)cc2)c2ccc(-c3nc4ccccc4s3)cc2)cc2ccc(-c3cccc4cc(C#N)c(C#N)cc34)cc12]

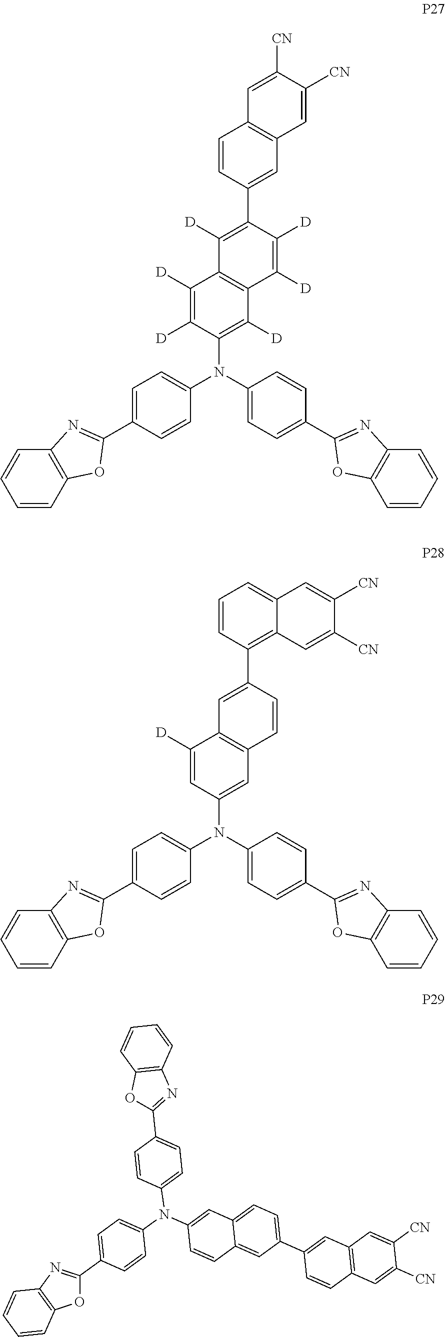 [SMILES: [2H]c1c(N(c2ccc(-c3nc4ccccc4o3)cc2)c2ccc(-c3nc4ccccc4o3)cc2)c([2H])c2c([2H])c([2H])c(-c3ccc4cc(C#N)c(C#N)cc4c3)c([2H])c2c1[2H] ; [2H]c1cc(N(c2ccc(-c3nc4ccccc4o3)cc2)c2ccc(-c3nc4ccccc4o3)cc2)cc2ccc(-c3cccc4cc(C#N)c(C#N)cc34)cc12 ; N#Cc1cc2ccc(-c3ccc4cc(N(c5ccc(-c6nc7ccccc7o6)cc5)c5ccc(-c6nc7ccccc7o6)cc5)ccc4c3)cc2cc1C#N]

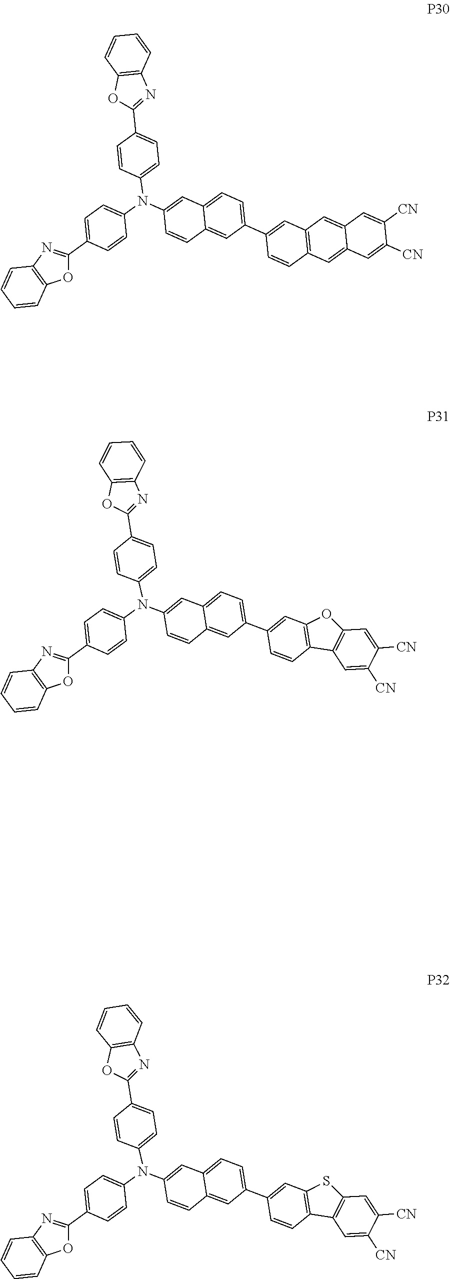

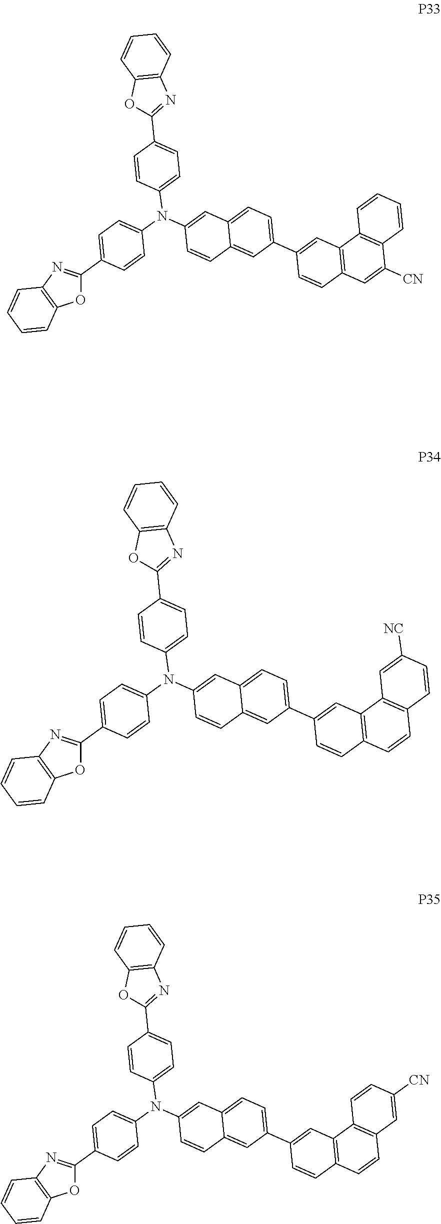 [SMILES: N#Cc1cc2ccc(-c3ccc4cc(N(c5ccc(-c6nc7ccccc7o6)cc5)c5ccc(-c6nc7ccccc7o6)cc5)ccc4c3)cc2c2ccccc12 ; N#Cc1ccc2ccc3ccc(-c4ccc5cc(N(c6ccc(-c7nc8ccccc8o7)cc6)c6ccc(-c7nc8ccccc8o7)cc6)ccc5c4)cc3c2c1 ; N#Cc1ccc2c(ccc3ccc(-c4ccc5cc(N(c6ccc(-c7nc8ccccc8o7)cc6)c6ccc(-c7nc8ccccc8o7)cc6)ccc5c4)cc32)c1]

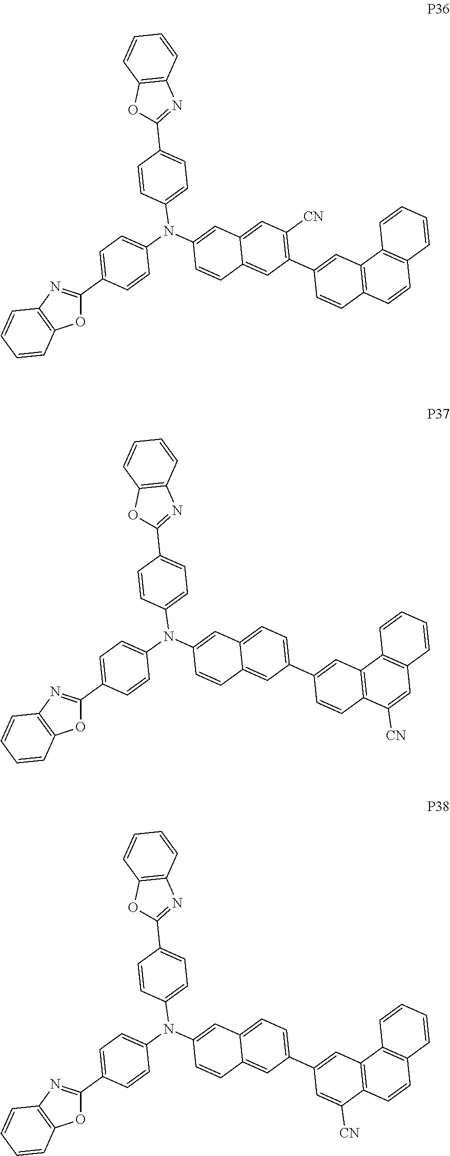 [SMILES: N#Cc1cc2cc(N(c3ccc(-c4nc5ccccc5o4)cc3)c3ccc(-c4nc5ccccc5o4)cc3)ccc2cc1-c1ccc2ccc3ccccc3c2c1 ; N#Cc1cc2ccccc2c2cc(-c3ccc4cc(N(c5ccc(-c6nc7ccccc7o6)cc5)c5ccc(-c6nc7ccccc7o6)cc5)ccc4c3)ccc12 ; N#Cc1cc(-c2ccc3cc(N(c4ccc(-c5nc6ccccc6o5)cc4)c4ccc(-c5nc6ccccc6o5)cc4)ccc3c2)cc2c1ccc1ccccc12]

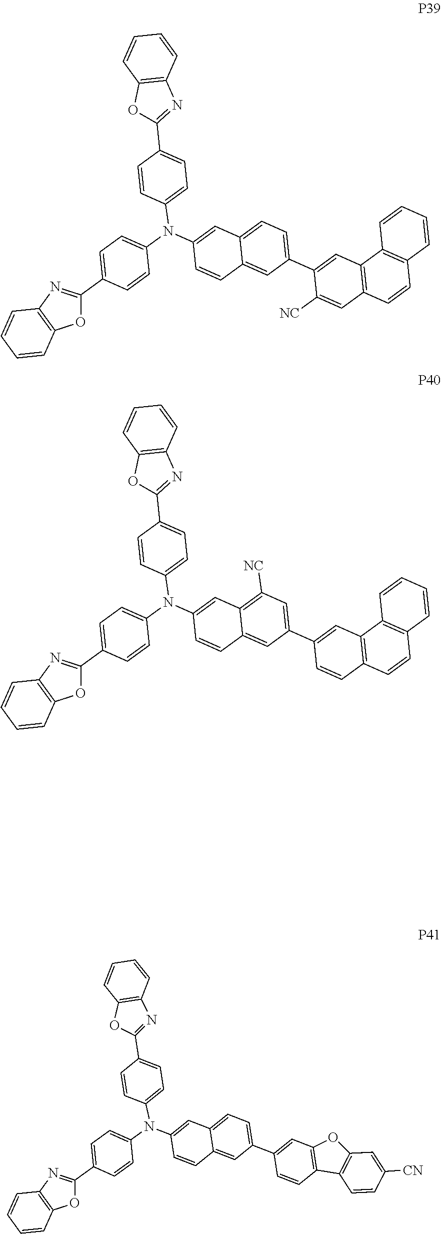 [SMILES: N#Cc1cc2ccc3ccccc3c2cc1-c1ccc2cc(N(c3ccc(-c4nc5ccccc5o4)cc3)c3ccc(-c4nc5ccccc5o4)cc3)ccc2c1 ; N#Cc1cc(-c2ccc3ccc4ccccc4c3c2)cc2ccc(N(c3ccc(-c4nc5ccccc5o4)cc3)c3ccc(-c4nc5ccccc5o4)cc3)cc12 ; N#Cc1ccc2c(c1)oc1cc(-c3ccc4cc(N(c5ccc(-c6nc7ccccc7o6)cc5)c5ccc(-c6nc7ccccc7o6)cc5)ccc4c3)ccc12]

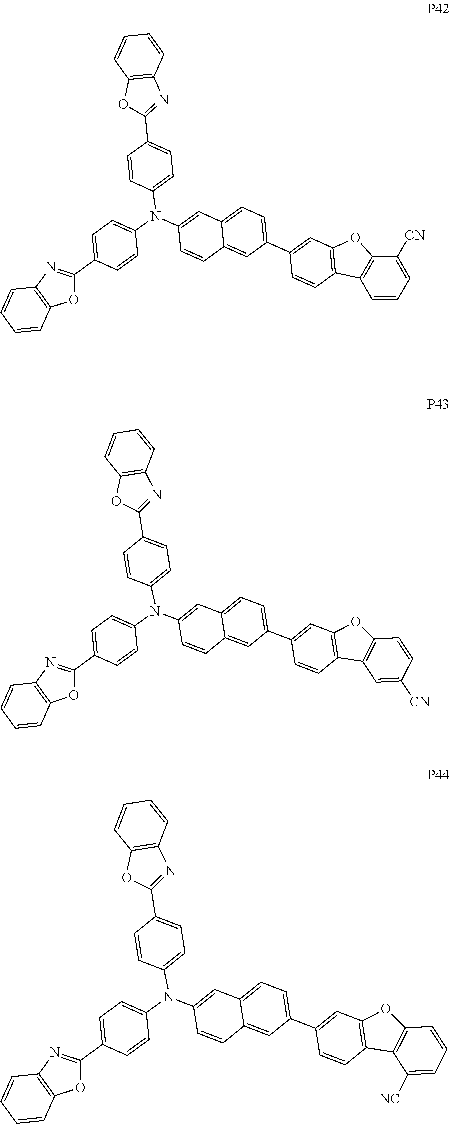 [SMILES: N#Cc1cccc2c1oc1cc(-c3ccc4cc(N(c5ccc(-c6nc7ccccc7o6)cc5)c5ccc(-c6nc7ccccc7o6)cc5)ccc4c3)ccc12 ; N#Cc1ccc2oc3cc(-c4ccc5cc(N(c6ccc(-c7nc8ccccc8o7)cc6)c6ccc(-c7nc8ccccc8o7)cc6)ccc5c4)ccc3c2c1 ; N#Cc1cccc2oc3cc(-c4ccc5cc(N(c6ccc(-c7nc8ccccc8o7)cc6)c6ccc(-c7nc8ccccc8o7)cc6)ccc5c4)ccc3c12]

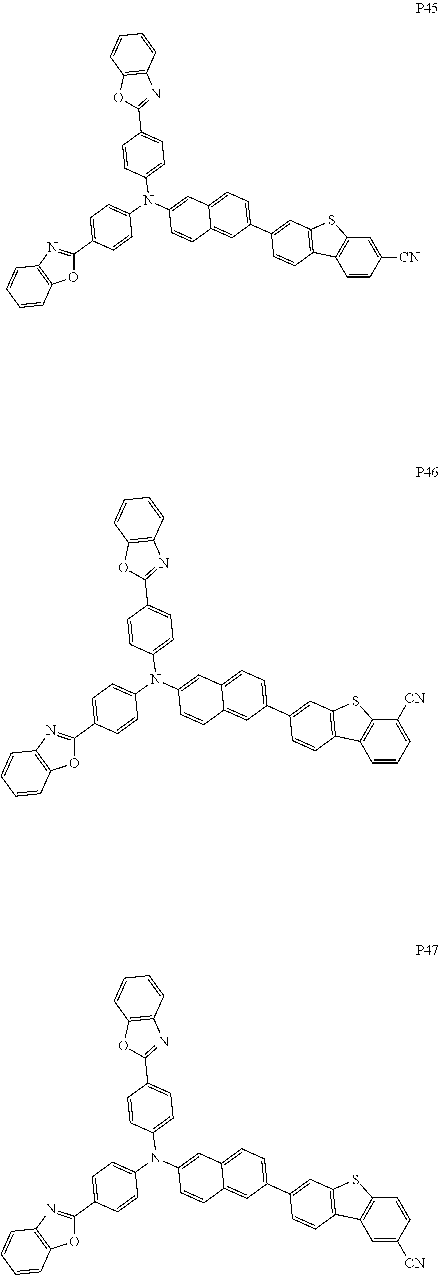 [SMILES: N#Cc1ccc2c(c1)sc1cc(-c3ccc4cc(N(c5ccc(-c6nc7ccccc7o6)cc5)c5ccc(-c6nc7ccccc7o6)cc5)ccc4c3)ccc12 ; N#Cc1cccc2c1sc1cc(-c3ccc4cc(N(c5ccc(-c6nc7ccccc7o6)cc5)c5ccc(-c6nc7ccccc7o6)cc5)ccc4c3)ccc12 ; N#Cc1ccc2sc3cc(-c4ccc5cc(N(c6ccc(-c7nc8ccccc8o7)cc6)c6ccc(-c7nc8ccccc8o7)cc6)ccc5c4)ccc3c2c1]

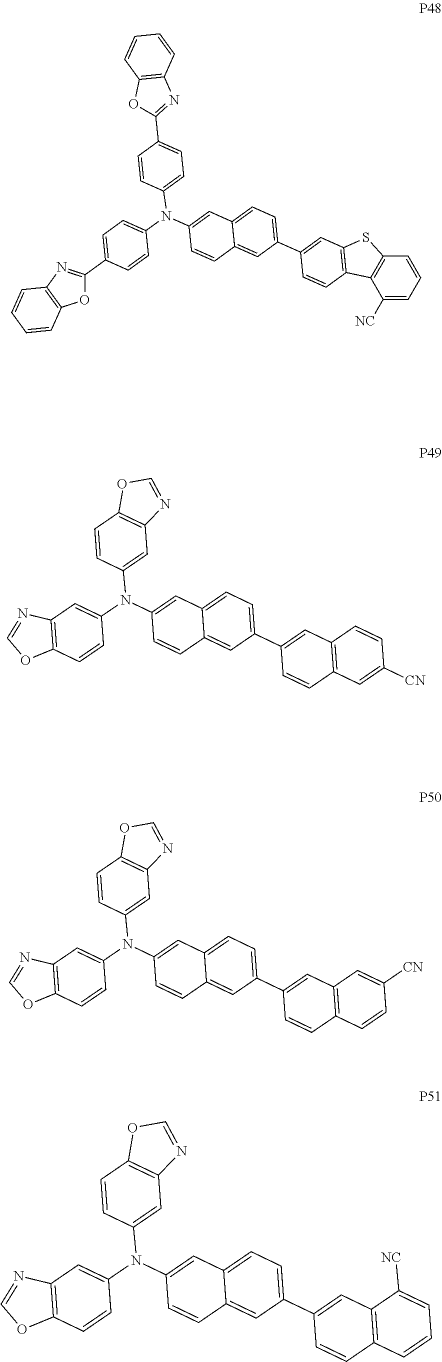 [SMILES: N#Cc1cccc2sc3cc(-c4ccc5cc(N(c6ccc(-c7nc8ccccc8o7)cc6)c6ccc(-c7nc8ccccc8o7)cc6)ccc5c4)ccc3c12 ; N#Cc1ccc2cc(-c3ccc4cc(N(c5ccc6ocnc6c5)c5ccc6ocnc6c5)ccc4c3)ccc2c1 ; N#Cc1ccc2ccc(-c3ccc4cc(N(c5ccc6ocnc6c5)c5ccc6ocnc6c5)ccc4c3)cc2c1 ; N#Cc1cccc2ccc(-c3ccc4cc(N(c5ccc6ocnc6c5)c5ccc6ocnc6c5)ccc4c3)cc12]

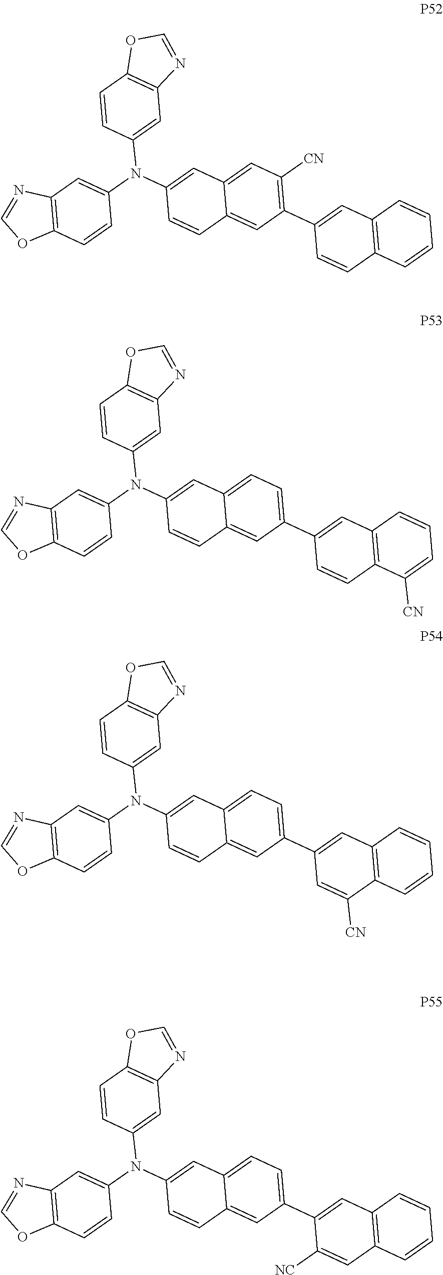 [SMILES: N#Cc1cc2cc(N(c3ccc4ocnc4c3)c3ccc4ocnc4c3)ccc2cc1-c1ccc2ccccc2c1 ; N#Cc1cccc2cc(-c3ccc4cc(N(c5ccc6ocnc6c5)c5ccc6ocnc6c5)ccc4c3)ccc12 ; N#Cc1cc(-c2ccc3cc(N(c4ccc5ocnc5c4)c4ccc5ocnc5c4)ccc3c2)cc2ccccc12 ; N#Cc1cc2ccccc2cc1-c1ccc2cc(N(c3ccc4ocnc4c3)c3ccc4ocnc4c3)ccc2c1]

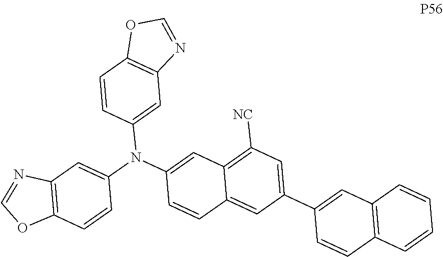 [SMILES: N#Cc1cc(-c2ccc3ccccc3c2)cc2ccc(N(c3ccc4ocnc4c3)c3ccc4ocnc4c3)cc12]

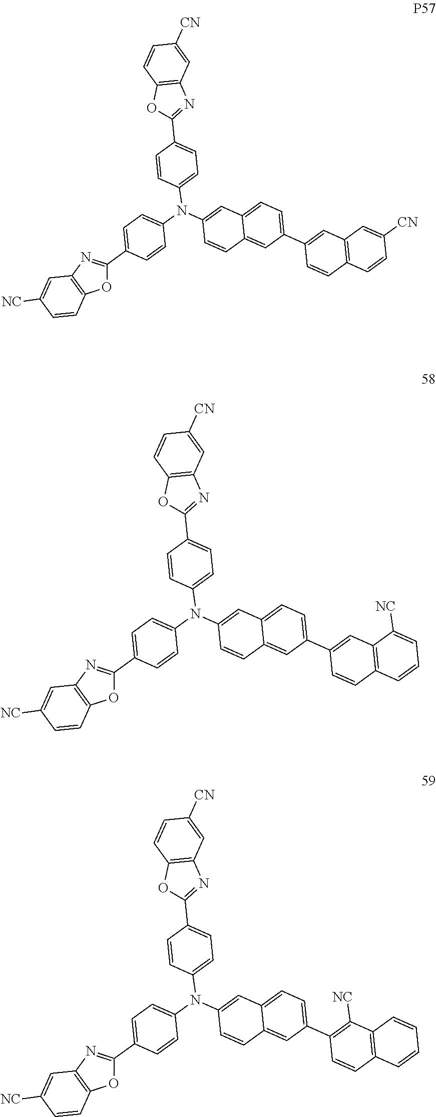 [SMILES: N#Cc1ccc2ccc(-c3ccc4cc(N(c5ccc(-c6nc7cc(C#N)ccc7o6)cc5)c5ccc(-c6nc7cc(C#N)ccc7o6)cc5)ccc4c3)cc2c1 ; N#Cc1ccc2oc(-c3ccc(N(c4ccc(-c5nc6cc(C#N)ccc6o5)cc4)c4ccc5cc(-c6ccc7cccc(C#N)c7c6)ccc5c4)cc3)nc2c1 ; N#Cc1ccc2oc(-c3ccc(N(c4ccc(-c5nc6cc(C#N)ccc6o5)cc4)c4ccc5cc(-c6ccc7ccccc7c6C#N)ccc5c4)cc3)nc2c1]

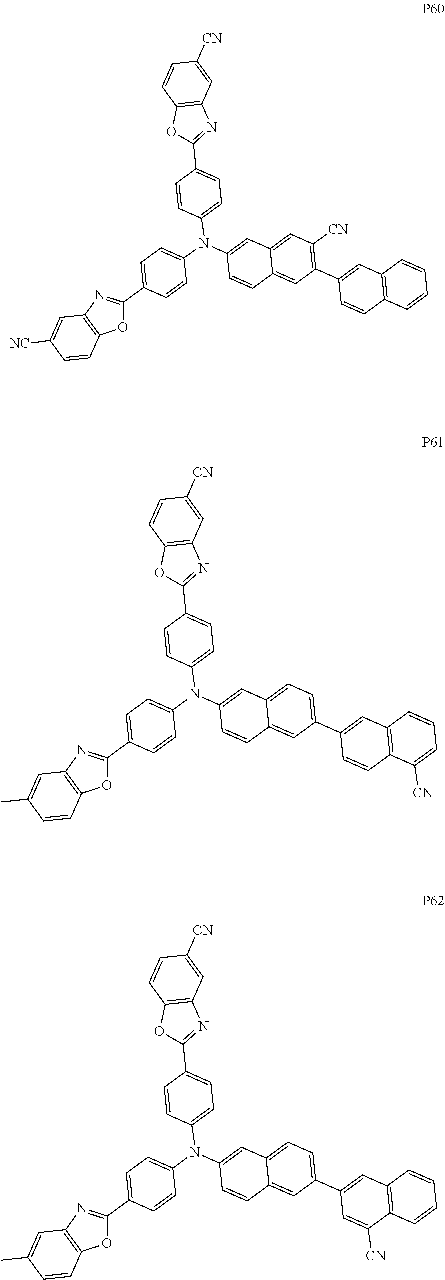 [SMILES: N#Cc1ccc2oc(-c3ccc(N(c4ccc(-c5nc6cc(C#N)ccc6o5)cc4)c4ccc5cc(-c6ccc7ccccc7c6)c(C#N)cc5c4)cc3)nc2c1 ; Cc1ccc2oc(-c3ccc(N(c4ccc(-c5nc6cc(C#N)ccc6o5)cc4)c4ccc5cc(-c6ccc7c(C#N)cccc7c6)ccc5c4)cc3)nc2c1 ; Cc1ccc2oc(-c3ccc(N(c4ccc(-c5nc6cc(C#N)ccc6o5)cc4)c4ccc5cc(-c6cc(C#N)c7ccccc7c6)ccc5c4)cc3)nc2c1]

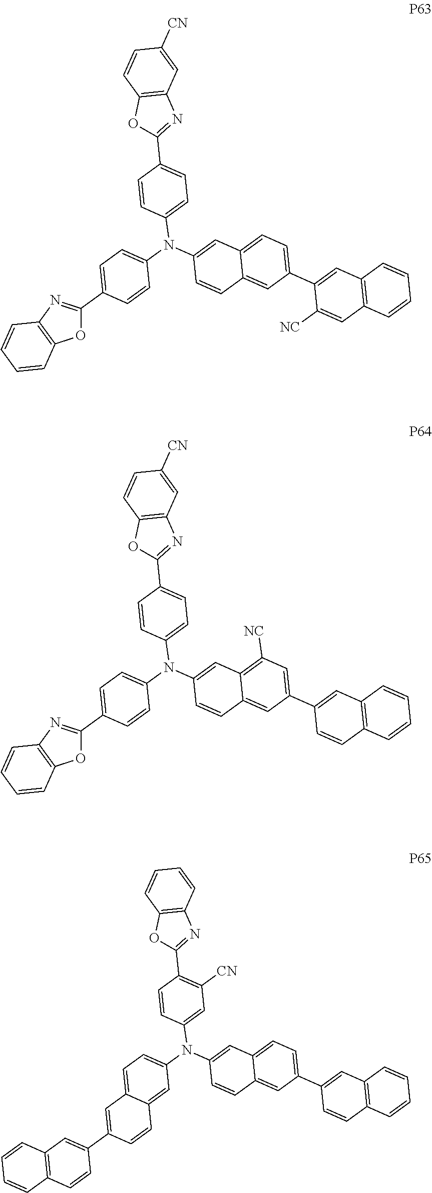 [SMILES: N#Cc1ccc2oc(-c3ccc(N(c4ccc(-c5nc6ccccc6o5)cc4)c4ccc5cc(-c6cc7ccccc7cc6C#N)ccc5c4)cc3)nc2c1 ; N#Cc1ccc2oc(-c3ccc(N(c4ccc(-c5nc6ccccc6o5)cc4)c4ccc5cc(-c6ccc7ccccc7c6)cc(C#N)c5c4)cc3)nc2c1 ; N#Cc1cc(N(c2ccc3cc(-c4ccc5ccccc5c4)ccc3c2)c2ccc3cc(-c4ccc5ccccc5c4)ccc3c2)ccc1-c1nc2ccccc2o1]

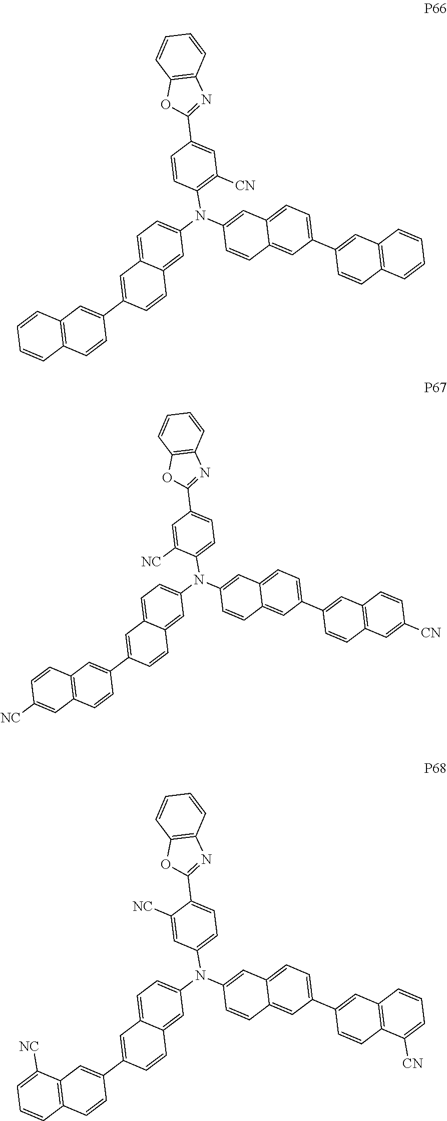 [SMILES: N#Cc1cc(-c2nc3ccccc3o2)ccc1N(c1ccc2cc(-c3ccc4ccccc4c3)ccc2c1)c1ccc2cc(-c3ccc4ccccc4c3)ccc2c1 ; N#Cc1ccc2cc(-c3ccc4cc(N(c5ccc6cc(-c7ccc8cc(C#N)ccc8c7)ccc6c5)c5ccc(-c6nc7ccccc7o6)cc5C#N)ccc4c3)ccc2c1 ; N#Cc1cc(N(c2ccc3cc(-c4ccc5c(C#N)cccc5c4)ccc3c2)c2ccc3cc(-c4ccc5cccc(C#N)c5c4)ccc3c2)ccc1-c1nc2ccccc2o1]

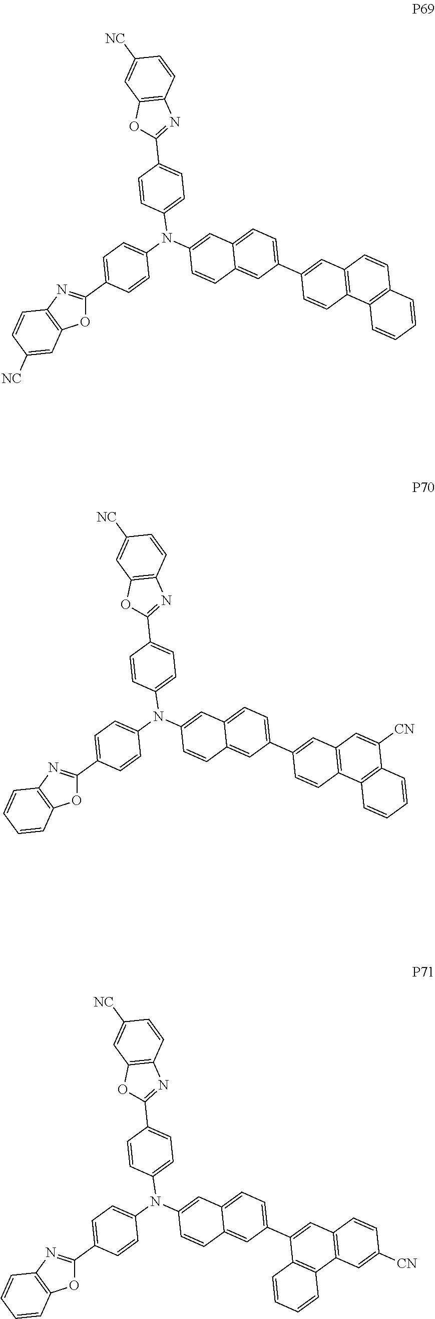 [SMILES: N#Cc1ccc2nc(-c3ccc(N(c4ccc(-c5nc6ccc(C#N)cc6o5)cc4)c4ccc5cc(-c6ccc7c(ccc8ccccc87)c6)ccc5c4)cc3)oc2c1 ; N#Cc1ccc2nc(-c3ccc(N(c4ccc(-c5nc6ccccc6o5)cc4)c4ccc5cc(-c6ccc7c(c6)cc(C#N)c6ccccc67)ccc5c4)cc3)oc2c1 ; N#Cc1ccc2nc(-c3ccc(N(c4ccc(-c5nc6ccccc6o5)cc4)c4ccc5cc(-c6cc7ccc(C#N)cc7c7ccccc67)ccc5c4)cc3)oc2c1]

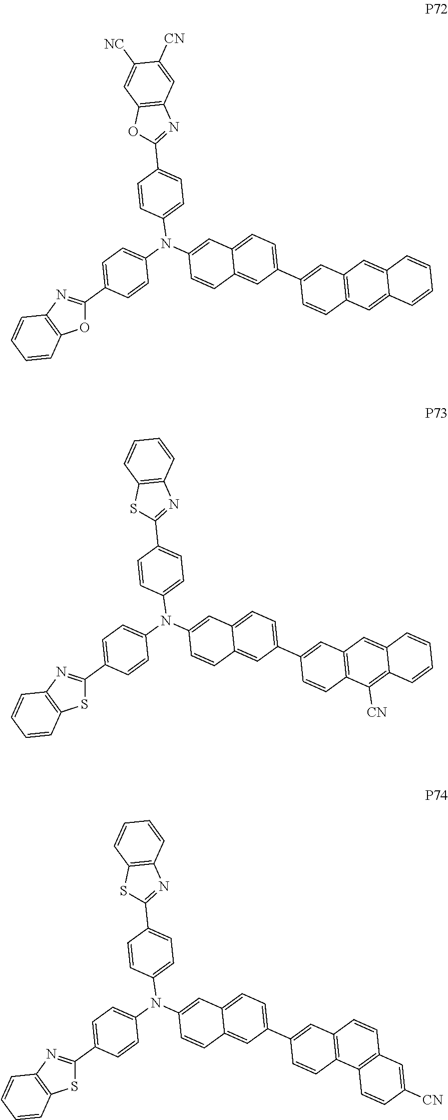 [SMILES: N#Cc1cc2nc(-c3ccc(N(c4ccc(-c5nc6ccccc6o5)cc4)c4ccc5cc(-c6ccc7cc8ccccc8cc7c6)ccc5c4)cc3)oc2cc1C#N ; N#Cc1c2ccccc2cc2cc(-c3ccc4cc(N(c5ccc(-c6nc7ccccc7s6)cc5)c5ccc(-c6nc7ccccc7s6)cc5)ccc4c3)ccc12 ; N#Cc1ccc2c(ccc3cc(-c4ccc5cc(N(c6ccc(-c7nc8ccccc8s7)cc6)c6ccc(-c7nc8ccccc8s7)cc6)ccc5c4)ccc32)c1]

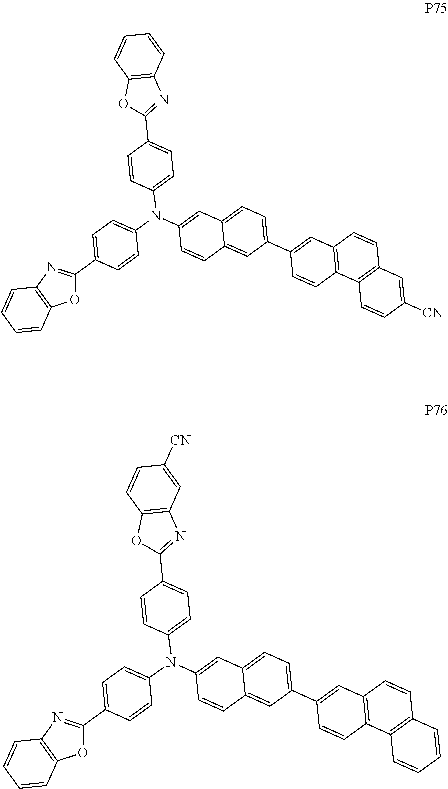 [SMILES: N#Cc1ccc2c(ccc3cc(-c4ccc5cc(N(c6ccc(-c7nc8ccccc8o7)cc6)c6ccc(-c7nc8ccccc8o7)cc6)ccc5c4)ccc32)c1 ; N#Cc1ccc2oc(-c3ccc(N(c4ccc(-c5nc6ccccc6o5)cc4)c4ccc5cc(-c6ccc7c(ccc8ccccc87)c6)ccc5c4)cc3)nc2c1]

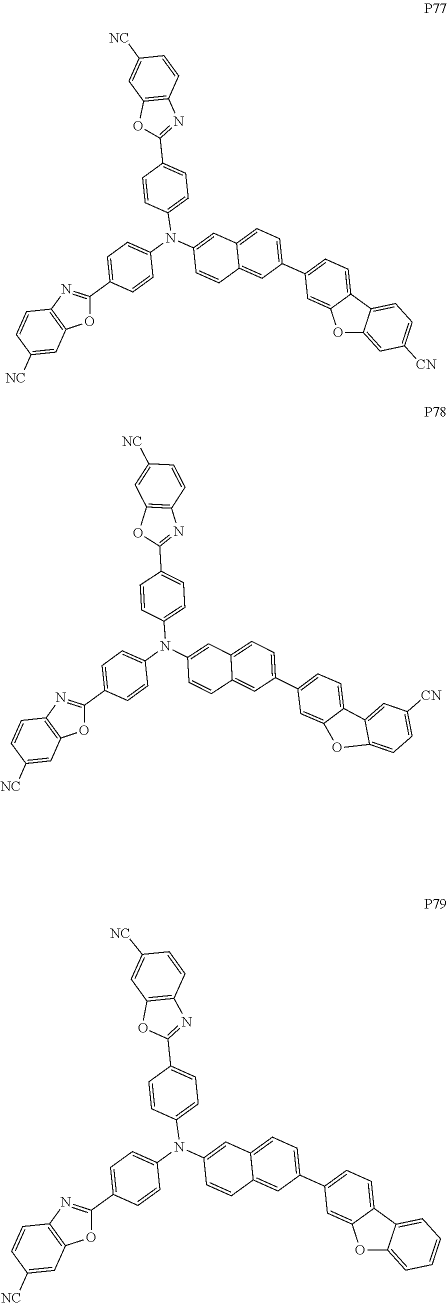

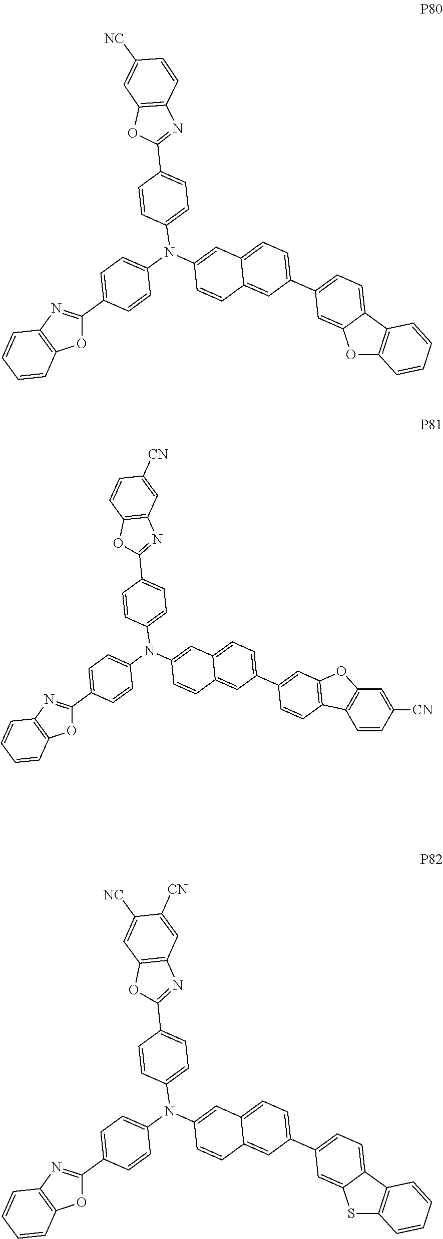

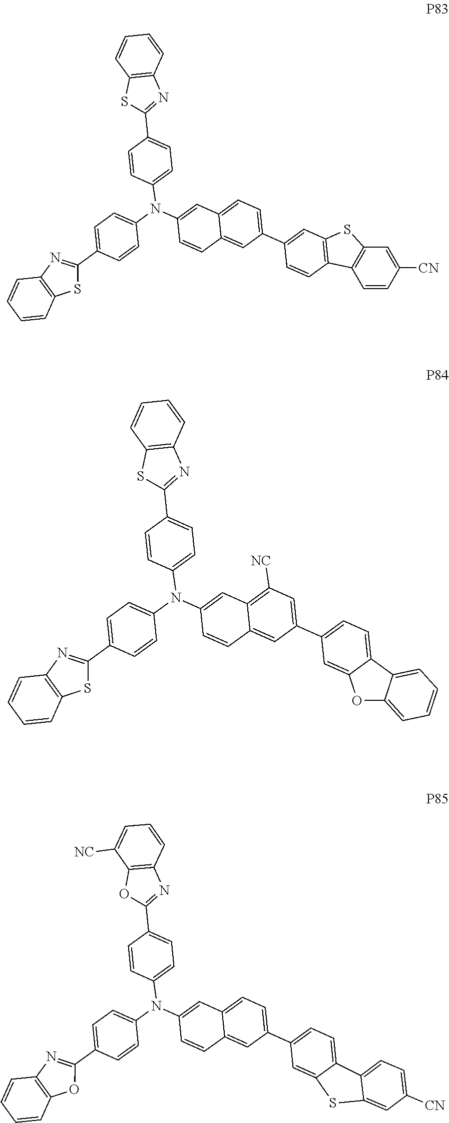

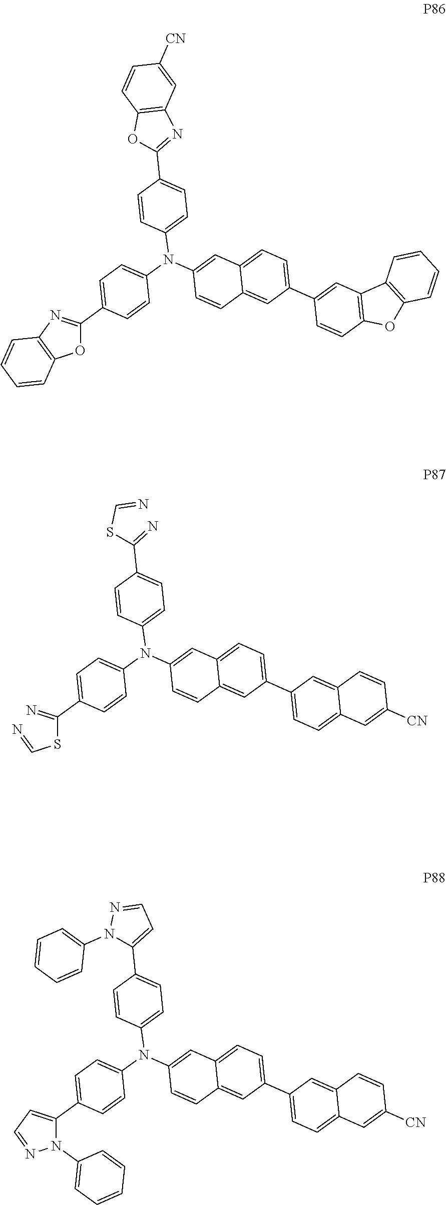

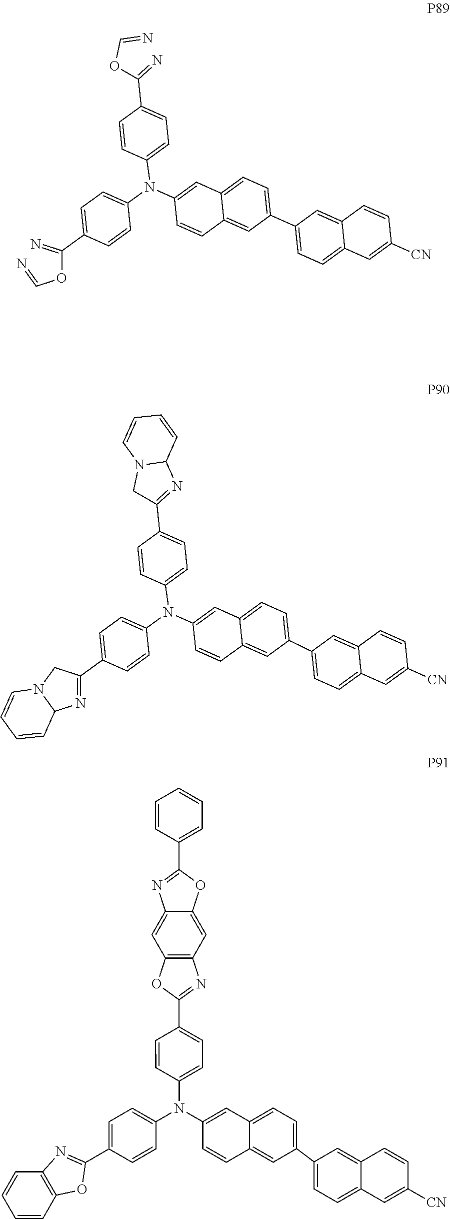 [SMILES: N#Cc1ccc2cc(-c3ccc4cc(N(c5ccc(C(=N)OC=N)cc5)c5ccc(C(=N)OC=N)cc5)ccc4c3)ccc2c1 ; N#Cc1ccc2cc(-c3ccc4cc(N(c5ccc(C6=NC7C=CC=CN7C6)cc5)c5ccc(C6=NC7C=CC=CN7C6)cc5)ccc4c3)ccc2c1 ; N#Cc1ccc2cc(-c3ccc4cc(N(c5ccc(-c6nc7ccccc7o6)cc5)c5ccc(-c6nc7cc8oc(-c9ccccc9)nc8cc7o6)cc5)ccc4c3)ccc2c1]

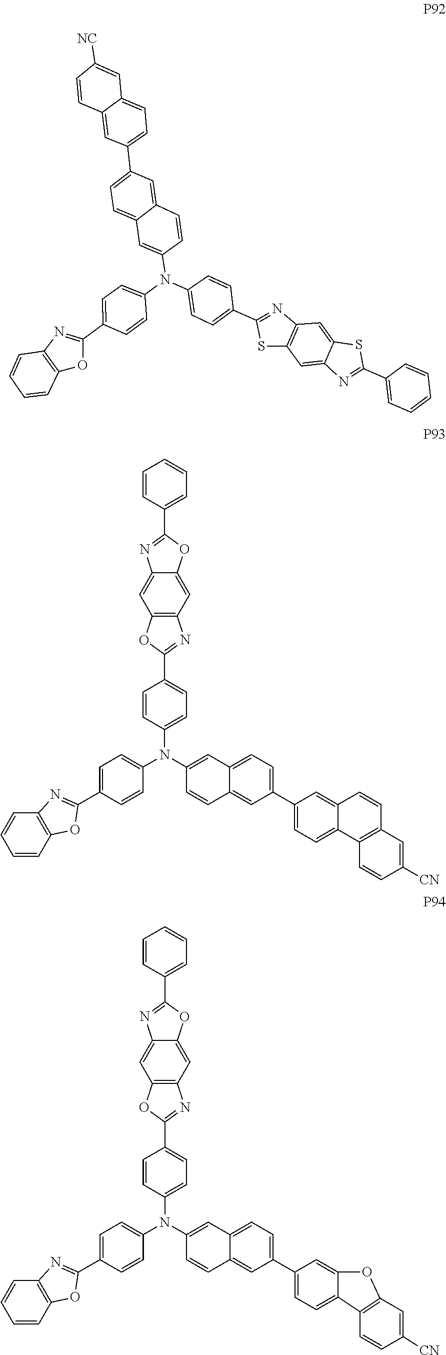 [SMILES: N#Cc1ccc2cc(-c3ccc4cc(N(c5ccc(-c6nc7ccccc7o6)cc5)c5ccc(-c6nc7cc8sc(-c9ccccc9)nc8cc7s6)cc5)ccc4c3)ccc2c1 ; N#Cc1ccc2c(ccc3cc(-c4ccc5cc(N(c6ccc(-c7nc8ccccc8o7)cc6)c6ccc(-c7nc8cc9oc(-c%10ccccc%10)nc9cc8o7)cc6)ccc5c4)ccc32)c1 ; N#Cc1ccc2c(c1)oc1cc(-c3ccc4cc(N(c5ccc(-c6nc7ccccc7o6)cc5)c5ccc(-c6nc7cc8oc(-c9ccccc9)nc8cc7o6)cc5)ccc4c3)ccc12]

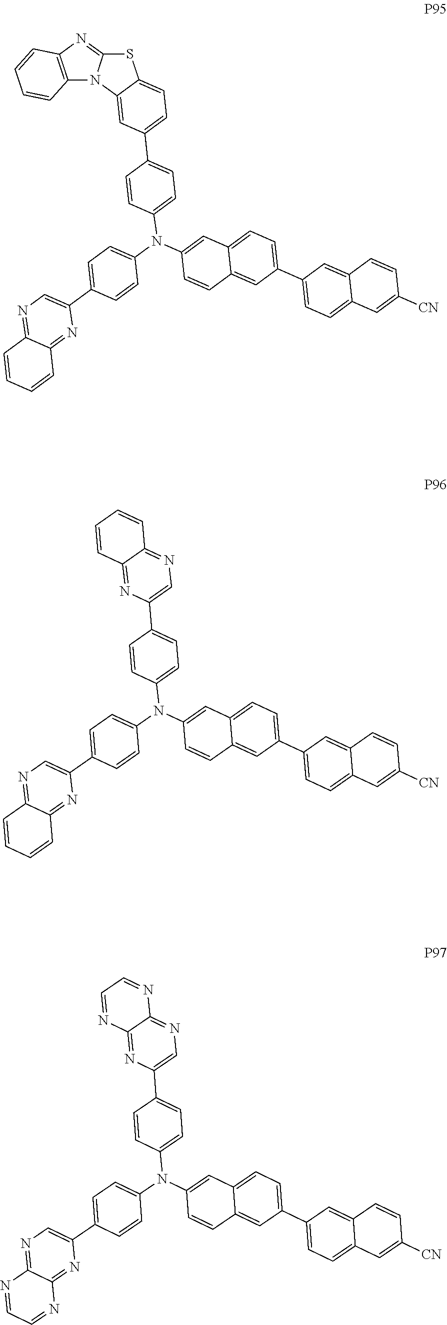 [SMILES: N#Cc1ccc2cc(-c3ccc4cc(N(c5ccc(-c6ccc7sc8nc9ccccc9n8c7c6)cc5)c5ccc(-c6cnc7ccccc7n6)cc5)ccc4c3)ccc2c1 ; N#Cc1ccc2cc(-c3ccc4cc(N(c5ccc(-c6cnc7ccccc7n6)cc5)c5ccc(-c6cnc7ccccc7n6)cc5)ccc4c3)ccc2c1 ; N#Cc1ccc2cc(-c3ccc4cc(N(c5ccc(-c6cnc7nccnc7n6)cc5)c5ccc(-c6cnc7nccnc7n6)cc5)ccc4c3)ccc2c1]

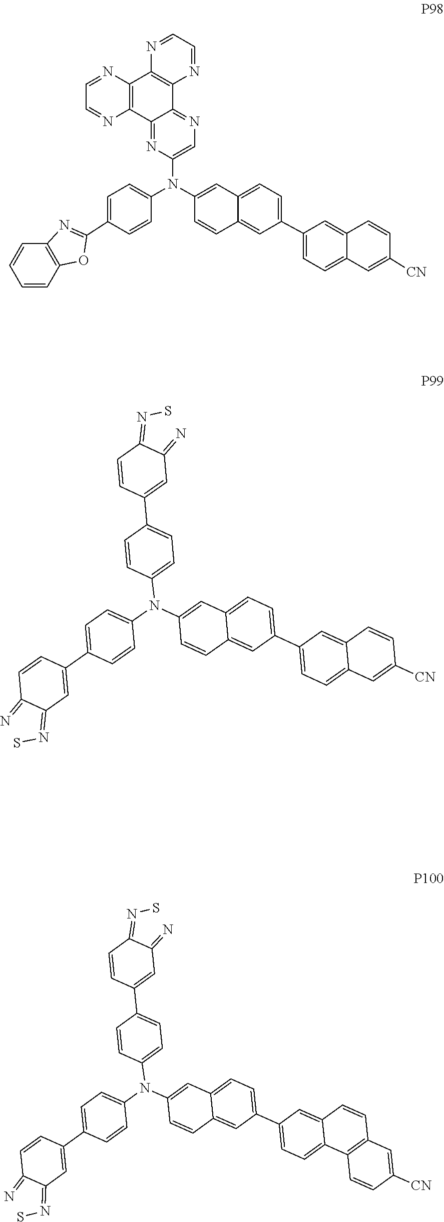 [SMILES: N#Cc1ccc2cc(-c3ccc4cc(N(c5ccc(-c6nc7ccccc7o6)cc5)c5cnc6c7nccnc7c7nccnc7c6n5)ccc4c3)ccc2c1 ; N#Cc1ccc2cc(-c3ccc4cc(N(c5ccc(C6=CC(=N)/C(=N\S)C=C6)cc5)c5ccc(C6=C/C(=N/S)C(=N)C=C6)cc5)ccc4c3)ccc2c1 ; N#Cc1ccc2c(ccc3cc(-c4ccc5cc(N(c6ccc(C7=CC(=N)/C(=N\S)C=C7)cc6)c6ccc(C7=C/C(=N/S)C(=N)C=C7)cc6)ccc5c4)ccc32)c1]

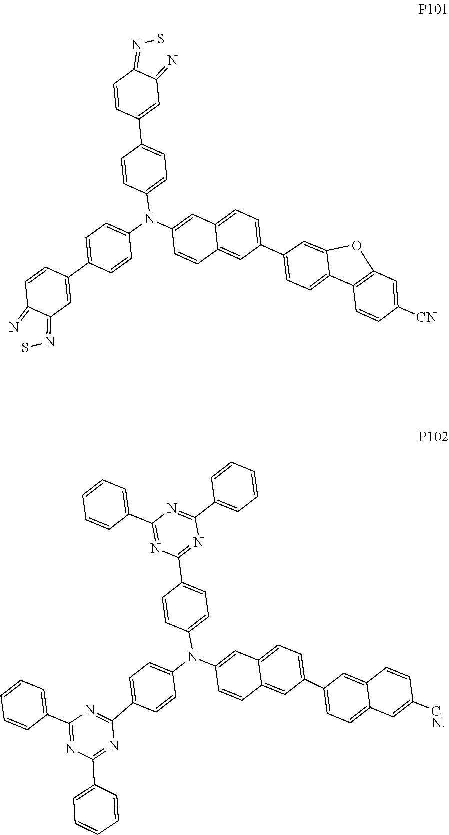 [SMILES: N#Cc1ccc2c(c1)oc1cc(-c3ccc4cc(N(c5ccc(C6=CC(=N)/C(=N\S)C=C6)cc5)c5ccc(C6=C/C(=N/S)C(=N)C=C6)cc5)ccc4c3)ccc12 ; N#Cc1ccc2cc(-c3ccc4cc(N(c5ccc(-c6nc(-c7ccccc7)nc(-c7ccccc7)n6)cc5)c5ccc(-c6nc(-c7ccccc7)nc(-c7ccccc7)n6)cc5)ccc4c3)ccc2c1]

17. An organic electroluminescent device, comprising a first electrode layer, an organic function layer and a second electrode layer which are stacked in sequence;
wherein the organic function layer comprises any one or a combination of at least two of the compounds according to claim 1.

18. An organic electroluminescent device, comprising a first capping layer, a first electrode layer, an organic function layer and a second electrode layer which are stacked in sequence;
wherein the first capping layer comprises any one or a combination of at least two of the compounds according to claim 1.

19. The organic electroluminescent device according to claim 18, further comprising a second capping layer disposed on a side of the first capping layer facing away from the first electrode layer, wherein the second capping layer comprises a material containing lithium fluoride and/or a small organic molecule with a refractive index of 1.40-1.65.

20. A display panel, comprising the organic electroluminescent device according to claim 17.
